(19) United States
(12) Patent Application Publication
(10) Pub. No.: US 2025/0376659 A1
(43) Pub. Date: Dec. 11, 2025

(54) DERIVATION OF SOMATOTROPHS FROM STEM CELLS AND USES THEREOF

(71) Applicant: MEMORIAL SLOAN-KETTERING CANCER CENTER, New York, NY (US)

(72) Inventors: Viviane Tabar, New York, NY (US); Jinghua Piao, Long Island City, NY (US)

(73) Assignee: MEMORIAL SLOAN-KETTERING CANCER CENTER, New York, NY (US)

(21) Appl. No.: 19/012,073

(22) Filed: Jan. 7, 2025

Related U.S. Application Data
(63) Continuation of application No. 16/869,224, filed on May 7, 2020, now abandoned.
Application No. 16/869,224 is a continuation of application No. PCT/US2018/060585, filed on Nov. 13, 2018.
(60) Provisional application No. 62/584,299, filed on Nov. 10, 2017.

(51) Int. Cl. C12N 5/071; G01N 33/50

(52) U.S. Cl. CPC C12N 5/0616; G01N 33/5008; C12N 2501/105; C12N 2501/119; C12N 2501/15; C12N 2501/155; C12N 2501/305; C12N 2501/385; C12N 2501/392; C12N 2501/395; C12N 2501/41; C12N 2501/415

(57) ABSTRACT

The presently disclosed subject matter provides for in vitro methods of inducing differentiation of stem cells (e.g., human stem cells) into somatotrophs, and differentiated cells generated by such methods. The presently disclosed subject matter also provides for uses of such cells for treating growth hormone deficiency.

### CROSS-REFERENCE TO RELATED APPLICATION

This application is a continuation of U.S. application Ser. No. 16/869,224, filed on May 7, 2020, which is a continuation of International Patent Application No. PCT/US2018/060585, filed on Nov. 13, 2018, which claims priority to U.S. Provisional Application No. 62/584,299, filed on Nov. 10, 2017, the contents of each of which are incorporated by reference in their entirety, and to each of which priority is claimed.

### GRANT INFORMATION

This invention was made with government support under Grant No. NCI-1-R21-CA176700 from National Institutes of Health. The government has certain rights in the invention.

### 1. INTRODUCTION

The presently disclosed subject matter relates to somatotrophs derived from stem cells (e.g., human stem cells), and uses thereof for cell-based treatment and drug discovery in growth-hormone (GH) deficiency.

### 2. BACKGROUND OF THE INVENTION

Growth hormone (GH) is produced in the anterior pituitary gland and has a profound effect especially in children. GH's effect on growth and metabolism is mediated by direct GH effects and indirect effects through the stimulation of Insulin-like growth factor 1 and 2 (IGF-1 and IGF-2) production. The main target tissues are bone, fat and muscle. Low GH levels result in decreased bone mineral density, muscle strength, and increase in cholesterol. In children, it will result in dwarfism.

Growth hormone deficiency includes congenital growth hormone deficiency and acquired growth hormone deficiency. Congenital growth hormone deficiency is due to mutations of genes involved in growth hormone development. Acquired growth hormone deficiency can be induced by tumor, surgery, injuries in the hypothalamic-pituitary region, etc. Congenital growth hormone deficiency can be divided into two categories: combined pituitary hormone deficiency (CPHD) and isolated growth hormone deficiency (IGHD) Types. Genetic mutations that can cause CPHD include POU1F1 mutation (CPHD1), PROP-1 mutation (CPHD2; most common, 12-55%), LHX3 mutation (CPHD3), and LHX4 mutation (CPHD4). Genetic mutations that can cause IGHD types include GH1 mutation (Types IA and II), GH1 or Growth-hormone-releasing hormone receptor (GHRHR) mutation (Type IB), and Bruton tyrosine kinase (BTK) mutation (Type III). Although numerous monogenic causes of growth disorders have been identified, most of the patients with IGHD/CPHD remain with an unexplained etiology as shown by the relatively low mutation detection rate. In addition, acquired etiologies of growth hormone and other pituitary hormones are multiple and affect a large number of patients.

The role of regenerative approaches to growth hormone deficiency has received very little attention despite the prevalence of the condition (45.5 per 100,000 people) and the large number of patients requiring pituitary hormone replacement. Current methods of treatment for growth hormone deficiency include life-long hormone replacement therapies, a suboptimal solution, since static delivery of these molecules is a poor substitute for normal pituitary gland features such as the dynamic secretion of hormones in response to circadian patterns, feedback mechanisms or stressful conditions. Treatment can be prohibitively expensive with costs of growth hormone replacement alone exceeding $50,000 per year. Thus, there are needs for new therapies for growth hormone deficiency.

### 3. SUMMARY OF THE INVENTION

The presently disclosed subject matter relates to somatotrophs (e.g., GH-producing somatotrophs) derived from stem cells (e.g., human stem cells), e.g., by in vitro differentiation.

In certain embodiments, the differentiation of stem cells to GH-producing somatotrophs includes three phases: (a) in vitro differentiation of stem cells to pituitary progenitors (also referred to as “pituitary precursors”), (b) in vitro differentiation of pituitary progenitors to cells expressing Pit1 (Pit1⁺ cells); and (c) in vitro differentiation of Pit1⁺ cells to GH-producing somatotrophs.

The present disclosure provides in vitro methods for inducing differentiation of pituitary precursors. For example, the present disclosure provides in vitro methods for inducing differentiation of pituitary precursors to a cell population of differentiated cells, wherein at least about 50% of differentiated cells are somatotrophs that are capable of producing growth hormone (referred to as “GH-producing somatotrophs”). In certain embodiments, the method comprises contacting cells expressing one or more pituitary precursor marker with one or more dorsalizing agent and one or more ventralizing agent; and contacting the cells with one or more activator of Wingless (Wnt) signaling (referred to as “Wnt activator”) and one or more molecule that is capable of inducing growth hormone (GH) expression (referred to as “GH inducer”) to obtain a cell population of differentiated cells, wherein at least about 50% of differentiated cells are GH-producing somatotrophs.

In certain embodiments, the GH-producing somatotrophs comprise cells expressing a low level of GHRHR immunoreactivity (GHRHR^{low} cells), cells expressing a high level of GHRHR immunoreactivity (GHRHR^{high} cells), and a combination thereof.

In certain embodiments, the method comprises obtaining a cell population comprising at least about 50% GHRHR^{low} cells at least about 10 days from the initial contact of the cells expressing one or more pituitary precursor marker with the one or more dorsalizing agent. In certain embodiments, the method comprises obtaining a cell population comprising at least about 50% GHRHR^{low} cells about two weeks or three weeks from the initial contact of the cells expressing one or more pituitary precursor marker with the one or more dorsalizing agent. In certain embodiments, the method comprises obtaining a cell population comprising at least about 50% GHRHR^{low} cells about 15 days from the initial contact of the cells expressing one or more pituitary precursor marker with the one or more dorsalizing agent.

In certain embodiments, the method comprises obtaining a cell population comprising at least about 50% GHRHR^{high} cells at least about 10 days from the initial contact of the cells expressing one or more pituitary precursor marker with the one or more dorsalizing agent. In certain embodiments, the method comprises obtaining a cell population comprising at least about 50% GHRHR^{high} cells about three weeks from the initial contact of the cells expressing one or more pituitary precursor marker with the one or more dorsalizing agent. In certain embodiments, the method comprises obtaining a cell population comprising at least about 50% GHRHR^{high} cells about 25 days from the initial contact of the cells expressing one or more pituitary precursor marker with the one or more dorsalizing agent. In certain embodiments, the method comprises obtaining a cell population comprising at least about 50% GHRHR^{high} cells about 35 days from the initial contact of the cells expressing one or more pituitary precursor marker with the one or more dorsalizing agent.

In addition, the present disclosure provides in vitro methods for inducing differentiation of pituitary precursors to a cell population of differentiated cells, wherein at least about 50% of differentiated cells express Pit1. In certain embodiments, the method comprises contacting cells expressing one or more pituitary precursor marker with one or more dorsalizing agent and one or more ventralizing agent; and contacting the cells with one or more activator of Wnt signaling to obtain a cell population of differentiated cells, wherein at least about 50% of differentiated cells express Pit1. In certain embodiments, the method comprises obtaining the cell population at least about 5 days from the initial contact of the cells expressing one or more pituitary precursor marker with the one or more dorsalizing agent. In certain embodiments, the method comprises obtaining the cell population about 7 days or about 11 days from the initial contact of the cells expressing one or more pituitary precursor marker with the one or more dorsalizing agent.

Furthermore, the present disclosure provides in vitro methods for inducing differentiation of stem cells. For example, the present disclosure provides in vitro methods for inducing differentiation of stem cells to a cell population of differentiated cells, wherein at least about 50% of differentiated cells are GH-producing somatotrophs. In certain embodiments, the method comprises contacting stem cells with one or more BMP molecule and one or more inhibitor of TGFβ/Activin-Nodal signaling; contacting the cells with one or more activator of SHH signaling (referred to as “SHH activator”), one or more activator of FGF signaling referred to as “FGF activator”), one or more dorsalizing agent, one or more ventralizing agent, one or more Wnt activator and one or more GH inducer to obtain a cell population of differentiated cells, wherein at least about 50% of differentiated cells are GH-producing somatotrophs.

In certain embodiments, the GH-producing somatotrophs comprise cells expressing a low level of GHRHR immunoreactivity (GHRHR^{low} cells), cells expressing a high level of GHRHR immunoreactivity (GHRHR^{high} cells), and a combination thereof. In certain embodiments, the method comprises obtaining a cell population comprising at least about 50% GHRHR^{low} cells at least about 3 weeks from the initial contact of the stem cells with the one or more inhibitor of TGFβ/Activin-Nodal signaling. In certain embodiments, the method comprises obtaining a cell population comprising at least about 50% GHRHR^{low} cells about 24 days from the initial contact of the stem cells with the one or more inhibitor of TGFβ/Activin-Nodal signaling. In certain embodiments, the method comprises obtaining a cell population comprising at least about 50% GHRHR^{low} cells about 4 weeks from the initial contact of the stem cells with the one or more inhibitor of TGFβ/Activin-Nodal signaling.

In certain embodiments, the method comprises obtaining a cell population comprising at least about 50% GHRHR^{high} cells at least about four weeks from the initial contact of the stem cells with the one or more inhibitor of TGFβ/Activin-Nodal signaling. In certain embodiments, the method comprises obtaining a cell population comprising at least about 50% GHRHR^{high} cells about 6 weeks from the initial contact of the stem cells with the one or more inhibitor of TGFβ/Activin-Nodal signaling. In certain embodiments, the method comprises obtaining a cell population comprising at least about 50% GHRHR^{high} cells about 30 days from the initial contact of the stem cells with the one or more inhibitor of TGFβ/Activin-Nodal signaling. In certain embodiments, the method comprises obtaining a cell population comprising at least about 50% GHRHR^{high} cells 33 days from the initial contact of the stem cells with the one or more inhibitor of TGFβ/Activin-Nodal signaling.

In addition, the present disclosure provides in vitro methods for inducing differentiation of stem cells to a cell population of differentiated cells, wherein at least about 50% of differentiated cells express Pit1. In certain embodiments, the methods comprises contacting stem cells with one or more BMP molecule and one or more inhibitor of TGFβ/Activin-Nodal signaling; contacting the cells with one or more SHH activator, one or more FGF activator, one or more dorsalizing agent, one or more ventralizing agent, and one or more Wnt activator to obtain a cell population of differentiated cells, wherein at least about 50% of differentiated cells express Pit1.

In certain embodiments, the method comprises obtaining the cell population at least about 10 days from the initial contact of the stem cells with the one or more inhibitor of TGFβ/Activin-Nodal signaling. In certain embodiments, the method comprises obtaining the cell population at least about 15 days from the initial contact of the stem cells with the one or more inhibitor of TGFβ/Activin-Nodal signaling. In certain embodiments, the method comprises obtaining the cell population about 15 days from the initial contact of the stem cells with the one or more inhibitor of TGFβ/Activin-Nodal signaling. In certain embodiments, the method comprises obtaining the cell population about 20 days from the initial contact of the stem cells with the one or more inhibitor of TGFβ/Activin-Nodal signaling.

For the various methods disclosed herein, in certain embodiments, the initial contact of the cells with the one or more Wnt activator is at least about 2 days and/or no later than about 5 days from the initial contact of the cells expressing one or more pituitary precursor marker with the one or more dorsalizing agent. In certain embodiments, the initial contact of the cells with the one or more Wnt activator occurs on the same day as the initial contact of the cells expressing one or more pituitary precursor marker with the one or more dorsalizing agent. In certain embodiments, the initial contact of the cells with the one or more Wnt activator is about 4 days from the initial contact of the cells expressing one or more pituitary precursor marker with the one or more dorsalizing agent.

In certain embodiments, the method comprises contacting the cells with the one or more Wnt activator for at least about 5 days and/or up to about 15 days. In certain embodiments, the method comprises contacting the cells with the one or more Wnt activator for at least about 7 days.

In certain embodiments, the method comprises contacting the cells with the one or more dorsalizing agent for at least about 3 days and/or up to about 10 days. In certain embodiments, the method comprises contacting the cells with the one or more dorsalizing agent for about 4 days or about 7 days. In certain embodiments, the method comprises contacting the cells with the one or more ventralizing agent for at least about 5 days and/or up to about 15 days. In certain embodiments, the method comprises contacting the cells with the one or more ventralizing agent for at least about 7 days or about 11 days.

In certain embodiments, the method comprises contacting the cells expressing one or more pituitary precursor marker with one or more inhibitor of TGFβ/Activin-Nodal signaling. In certain embodiments, the method comprises contacting the cells with the one or more inhibitor of TGFβ/Activin-Nodal signaling for at least about 3 days and/or up to about 10 days. In certain embodiments, the method comprises contacting the cells with the one or more inhibitor of TGFβ/Activin-Nodal signaling for about 4 days or about 7 days. In certain embodiments, the method comprises contacting the cells with the one or more dorsalizing agent and the one or more inhibitor of TGFβ/Activin-Nodal signaling concurrently.

In certain embodiments, the method further comprises contacting the cells with one or more estrogen receptor (ER) agonist. In certain embodiments, the method comprises contacting the cells with one or more ER agonist and the one or more Wnt activator concurrently. In certain embodiments, the initial contact of the cells with the one or ER agonist is at least about 2 days and/or no later than about 5 days from the initial contact of the cells expressing one or more pituitary precursor marker with the one or more dorsalizing agent. In certain embodiments, the initial contact of the cells with the one or more ER agonist occurs on the same day as the initial contact of the cells expressing one or more pituitary precursor marker with the one or more dorsalizing agent. In certain embodiments, the initial contact of the cells with the one or more ER agonist is about 4 days from the initial contact of the cells expressing one or more pituitary precursor marker with the one or more dorsalizing agent. In certain embodiments, the method comprises contacting the cells with the one or more ER agonist for at least about 5 days and/or up to about 15 days. In certain embodiments, the method comprises contacting the cells with the one or more ER agonist for at least about 7 days.

In certain embodiments, the cells expressing one or more pituitary precursor marker are obtained by in vitro differentiation of stem cells. In certain embodiments, the in vitro differentiation of stem cells comprises contacting stem cells with one or more BMP molecule and one or more inhibitor of TGFβ/Activin-Nodal signaling, contacting the cells with one or more SHH activator and one, two, or more FGF activator.

In certain embodiments, the stem cells are contacted with the one or more BMP molecule for at least about 2 days and/or no more than about 4 days. In certain embodiments, the stem cells are contacted with the one or more inhibitor of TGFβ/Activin-Nodal signaling for at least about 5 days. In certain embodiments, the stem cells are contacted with the one or more inhibitor of TGFβ/Activin-Nodal signaling for about 8 days.

In certain embodiments, the stem cells are contacted with the one or more SHH activator and one, two, or more FGF activator for at least about 2 days. In certain embodiments, the stem cells are contacted with the one or more SHH activator and one, two, or more FGF activator for about 5 days.

In certain embodiments, the in vitro differentiation of stem cells further comprises contacting the cells with one or more inhibitor of SMAD signaling (referred to as “SMAD inhibitor”). In certain embodiments, the stem cells are contacted with the one or more SMAD inhibitor for at least about 2 days. In certain embodiments, the stem cells are contacted with the one or more inhibitor of SMAD signaling for about 5 days.

In certain embodiments, the various methods comprise differentiating cells expressing one or more pituitary precursor marker to cells expressing Pit1, differentiating the cells expressing Pit1 to GHRHR^{low} cells, and differentiating the GHRHR^{low} cells to GHRHR^{high} cells. In certain embodiments, the method comprises differentiating the stem cells to cells expressing one or more pituitary precursor marker, differentiating the cells expressing one or more pituitary precursor marker to cells expressing Pit1, differentiating the cells expressing Pit1 to GHRHR^{low} cells, and differentiating the GHRHR^{low} cells to GHRHR^{high} cells. In certain embodiments, the method comprises differentiating the stem cells to cells expressing one or more pituitary precursor marker, and differentiating the cells expressing one or more pituitary precursor marker to cells expressing Pit1.

In certain embodiments, the differentiation of the cells expressing Pit1 to GHRHR^{low} cells comprises contacting cells expressing Pit1 with a first combination of GH inducers. In certain embodiments, the first combination of GH inducers are selected from the group consisting of retinoic acid (RA), corticosteroids, thyroid hormones, GHRH signaling agonists, ER agonists, and Ghrelin signaling pathway agonists. In certain embodiments, the first combination of GH inducers comprises retinoic acid (RA), a corticosteroid, a thyroid hormone, and a GHRH signaling agonist. In certain embodiments, the first combination of GH inducers comprises RA, dexamethasone, T3, and GHRH. In certain embodiments, the first combination of GH inducers comprises retinoic acid (RA), a corticosteroid, a thyroid hormone, two GHRH signaling agonists, and an ER agonist. In certain embodiments, the first combination of GH inducers comprises RA, dexamethasone, T3, GHRH, CAMP, and DPN. In certain embodiments, the first combination of GH inducers comprises retinoic acid (RA), a corticosteroid, a thyroid hormone, a GHRH signaling agonist, an ER agonist, and a Ghrelin signaling pathway agonist. In certain embodiments, the first combination of GH inducers comprises RA, dexamethasone, T3, GHRH, DPN, and Ghrelin.

In certain embodiments, the method comprises contacting the cells expressing Pit1 with the first combination of GH inducers for at least about 3 days to obtain a cell population comprising at least about 50% GHRHR^{low} cells. In certain embodiments, the method comprises contacting the cells expressing Pit1 with the first combination of GH inducers for about 5 days to obtain a cell population comprising at least about 50% GHRHR^{low} cells. In certain embodiments, the method comprises contacting the cells expressing Pit1 with the first combination of GH inducers for about two weeks to obtain a cell population comprising at least about 50% GHRHR^{low} cells.

In certain embodiments, the differentiation of the GHRHR^{low} cells to GHRHR^{high} cells comprises contacting the GHRHR^{low} cells with a second combination of GH inducers. In certain embodiments, the second combination of GH inducers are selected from the group consisting of retinoic acid (RA), corticosteroids, thyroid hormones, GHRH signaling agonists, ER agonists, interleukins, and Ghrelin signaling pathway agonists. In certain embodiments, the second combination of GH inducers comprises retinoic acid (RA), a corticosteroid, a thyroid hormone, and a GHRH signaling agonist. In certain embodiments, the second combination of GH inducers comprises RA, dexamethasone, T3, and GHRH. In certain embodiments, the second combination of GH inducers comprises retinoic acid (RA), a corticosteroid, a thyroid hormone, two GHRH signaling agonists, and an ER agonist. In certain embodiments, the second combination of GH inducers comprises RA, dexamethasone, T3, GHRH, CAMP, and DPN. In certain embodiments, the second combination of GH inducers comprises a corticosteroid, a thyroid hormone, a GHRH signaling agonist, an ER agonist, an interleukin, and a Ghrelin signaling pathway agonist. In certain embodiments, the second combination of GH inducers comprises dexamethasone, T3, GHRH, IL-6, Ghrelin, and DPN.

In certain embodiments, the method comprises contacting the GHRHR^{low} cells with the second combination of GH inducers for at least about 5 days to obtain a cell population comprising at least about 50% GHRHR^{high} cells. In certain embodiments, the method comprises contacting the GHRHR^{low} cells with the second combination of GH inducers for about 10 days to obtain a cell population comprising at least about 50% GHRHR^{high} cells. In certain embodiments, the method comprises contacting the GHRHR^{low} cells with the second combination of GH inducers for about four weeks to obtain a cell population comprising at least about 50% GHRHR^{high} cells.

In certain embodiments, the presently disclosed subject matter provides in vitro methods for inducing differentiation of stem cells (e.g., human stem cells). In certain embodiments, the presently disclosed subject matter provides in vitro methods for inducing differentiation of stem cells into cells expressing one or more pituitary precursor marker (pituitary precursors). In certain embodiments, the methods comprise contacting a population of stem cells with effective amounts of one or more inhibitor of transforming growth factor beta (TGFβ)/Activin-Nodal signaling, effective amounts of one or more activator of BMP signaling, effective amounts of one or more activator of Sonic Hedgehog (SHH) signaling, and effective amounts of one, two or more activators of FGF signaling. In certain embodiments, the methods further comprise contacting the cells with one or more inhibitor of SMAD signaling. In certain embodiments, the activators of FGF signaling activate at least FGF8 and FGF10 signaling. In certain embodiments, the activators of FGF signaling activate at least FGF8, FGF10 and FGF18 signaling. In certain embodiments, the cells are contacted with the one or more activator of BMP signaling for at least about 2 days, or at least about 3 days. In certain embodiments, the cells are contacted with the one or more activator of BMP signaling for up to 3 days, up to 4 days, or up to 5 days. In certain embodiments, the cells are contacted with the one or more activator of SHH signaling and two or more activators of FGF signaling, and optionally one or more SMAD inhibitor at least about 3 days from the initial contact of the stem cells with the one or more inhibitor of TGFβ/Activin-Nodal signaling. In certain embodiments, the cells are contacted with the one or more activator of SHH signaling and two or more activators of FGF signaling, and optionally one or more SMAD inhibitor about 3 days or about 4 days from the initial contact of the stem cells with the one or more inhibitor of TGFβ/Activin-Nodal signaling. In certain embodiments, the cells are contacted for at least about 5 days and up to about 10 days with the one or more SHH activator, and the two or more FGF activators, and optionally the one or more SMAD inhibitor. In certain embodiments, the cells are contacted for at least about 9 days with the one or more SHH activator, and the two or more FGF activators, and optionally the one or more SMAD inhibitor. In certain embodiments, the cells are contacted for at least about 8 days with the one or more SHH activator, and the two or more FGF activators, and optionally the one or more SMAD inhibitor.

The pituitary precursors can be differentiated in vitro into cells expressing Pit1. In certain embodiments, the methods for differentiating pituitary precursors into a cell population comprising at least about 50% (e.g., at least about 70% or about 80%) cells expressing Pit1 comprise contacting a population of pituitary precursors with effective amounts of one or more dorsalizing agent, effective amounts of one or more ventralizing agent, and effective amounts of one or more Wnt activator. In certain embodiments, the methods further comprise contacting the pituitary precursors with effective amounts of one or more agonist of estrogen receptor (ER) (ER agonist). In certain embodiments, the cells are contacted with the one or more dorsalizing agent, the one or more ventralizing agent, and the one or more Wnt activator, and optionally the ER agonist for at least about 5 days and up to about 10 days. In certain embodiments, the cells are contacted with the one or more dorsalizing agent, the one or more ventralizing agent, and the one or more Wnt activator, and optionally the ER agonist for about 7 days. In certain embodiments, the cells are contacted with the one or more dorsalizing agent, the one or more ventralizing agent, and the one or more Wnt activator, and optionally the ER agonist for about 8 days.

The cells expressing Pit1 can be further differentiated in vitro into somatotrophs. In certain embodiments, the methods for differentiating cells expressing Pit1 into a cell population comprising at least about 50% (e.g., at least about 70% or about 80%) cells expressing one or more somatotroph marker comprise contacting the cells expressing Pit1 with one or more molecule that is capable of inducing growth hormone (GH) expression (GH inducer). In certain embodiments, the Pit1⁺ cells are contacted with one, two, three, four, five, or six GH inducers.

In certain embodiments, the presently disclosed subject matter provides for in vitro methods for inducing differentiation of stem cells (e.g., human stem cells) into cells expressing one or more somatotroph marker (somatotrophs). In certain embodiments, the in vitro method for inducing differentiation of stem cells into cells expressing one or more somatotroph marker comprise contacting a population of stem cells with (a) effective amounts of one or more inhibitor of transforming growth factor beta (TGFβ)/Activin-Nodal signaling, (b) effective amounts of one or more activator of BMP signaling, (c) effective amounts of one or more activator of Sonic Hedgehog (SHH) signaling, (d) effective amounts of two or more activators of FGF signaling, (e) effective amounts of one or more dorsalizing agent, (f) effective amounts of one or more ventralizing agent, and (g) effective amounts of one or more Wnt activator.

In certain embodiments, the cells are contacted with the one or more activator of BMP signaling for at least about 2 days, or at least about 3 days. In certain embodiments, the cells are contacted with the one or more activator of BMP signaling for up to 3 days, up to 4 days, or up to 5 days. In certain embodiments, the cells are contacted with the one or more activator of SHH signaling and two or more activators of FGF signaling at least about 3 days from the initial contact of the stem cells with the one or more inhibitor of TGFβ/Activin-Nodal signaling. In certain embodiments, the cells are contacted with the one or more activator of SHH signaling and two or more activators of FGF signaling about 3 days or about 4 days from the initial contact of the stem cells with the one or more inhibitor of TGFβ/Activin-Nodal signaling. In certain embodiments, the cells are contacted for at least about 5 days and up to about 10 days with the one or more SHH activator, and the two or more FGF activators. In certain embodiments, the cells are contacted for at least about 9 days with the one or more SHH activator, and the two or more FGF activators. In certain embodiments, the one or more activator of SHH signaling and two or more activators of FGF signaling are contacted with the cells concurrently.

In certain embodiments, the cells are contacted with the one or more dorsalizing agent, the one or more ventralizing agent, and the one or more Wnt activator for at least about 5 days and up to about 10 days. In certain embodiments, the cells are contacted with the one or more dorsalizing agent, the one or more ventralizing agent, and the one or more Wnt activator for about 7 days. In certain embodiments, the cells are contacted with the one or more dorsalizing agent, the one or more ventralizing agent, and the one or more Wnt activator for about 8 days. In certain embodiments, the cells are contacted with the one or more dorsalizing agent, the one or more ventralizing agent, and the one or more Wnt activator at least about 5 days and up to about 15 days from the initial contact of the stem cells with the one or more inhibitor of TGFβ/Activin-Nodal signaling. In certain embodiments, the cells are contacted with the one or more dorsalizing agent, the one or more ventralizing agent, and the one or more Wnt activator about 8 days from the initial contact of the stem cells with the one or more inhibitor of TGFβ/Activin-Nodal signaling. In certain embodiments, the cells are contacted with the one or more dorsalizing agent, the one or more ventralizing agent, and the one or more Wnt activator about 9 days from the initial contact of the stem cells with the one or more inhibitor of TGFβ/Activin-Nodal signaling. In certain embodiments, the one or more dorsalizing agent, the one or more ventralizing agent, and the one or more Wnt activator are contacted with the cells concurrently.

In certain embodiments, the methods further comprise contacting the cells with (h) effective amounts of one or more SMAD inhibitors. In certain embodiments, the one or more SMAD inhibitor is contacted with the cells concurrently with the one or more activator of SHH signaling and two or more activators of FGF signaling.

In certain embodiments, the methods further comprise contacting the cells with (i) effective amounts of one or more ER agonist. In certain embodiments, the one or more ER agonist is contacted with the cells concurrently with the one or more dorsalizing agent, the one or more ventralizing agent, and the one or more Wnt activator.

In certain embodiments, the cells are contacted with the one or more GH inducer at least about 10 days and up to about 25 days from the initial contact of the stem cells with the one or more inhibitor of TGFβ/Activin-Nodal signaling. In certain embodiments, the cells are contacted with the one or more GH inducer about 15 days from the initial contact of the stem cells with the one or more inhibitor of TGFβ/Activin-Nodal signaling. In certain embodiments, the cells are contacted with the one or more GH inducer about 16 days from the initial contact of the stem cells with the one or more inhibitor of TGFβ/Activin-Nodal signaling. In certain embodiments, the cells are contacted with the one or more GH inducer for at least about 10 days and up to about 10 weeks. In certain embodiments, the cells are contacted with the one or more GH inducer for at least about 2 weeks, at least about 4 weeks, or at least about 6 weeks. In certain embodiments, the cells are contacted with the one or more GH inducer for about 2 weeks. In certain embodiments, the cells are contacted with the one or more GH inducer for about 4 weeks. In certain embodiments, the cells are contacted with the one or more GH inducer for about 6 weeks.

In certain embodiments, the cell population comprises at least about 50% (e.g., about 70% or 80%) of cells expressing one or more somatotroph marker at least about 30 days from the initial contact of the stem cells with the one or more inhibitor of TGFβ/Activin-Nodal signaling.

In certain embodiments, the cells are contacted with effective amounts of the foregoing agents for a period of time such that at least 50% (e.g., about 70% or about 80%) of the cells express detectable levels of one or more somatotroph marker.

In certain embodiments, the one or more dorsalizing agent comprises one or more activator of FGF signaling. In certain embodiments, the one or more activator of FGF signaling is selected from the group consisting of FGF1, FGF2, FGF3, FGF4, FGF7, FGF8, FGF10, FGF18, derivatives thereof, and mixtures thereof. In certain embodiments, the one or more activator of FGF signaling comprises FGF8. In certain embodiments, the one or more activator of FGF signaling comprises FGF8 and FGF10. In certain embodiments, the one or more activator of FGF signaling comprises FGF8, FGF10, and FGF18.

In certain embodiments, the one or more ventralizing agent comprises one or more BMP molecule. In certain embodiments, the one or more BMP molecule is selected from the group consisting of BMP1, BMP2, BMP3, BMP4, BMP5, BMP6, BMP7, BMP8a, BMP8b, BMP10, BMP11, BMP15, derivatives thereof, and mixtures thereof. In certain embodiments, the one or more BMP molecule comprises BMP2. In certain embodiments, the one or more BMP molecule comprises BM4.

In certain embodiments, the one or more Wnt activator is selected from the group consisting of CHIR99021, Wnt-1, WNT3A, Wnt4, Wnt5a, WAY-316606, IQ1, QS11, SB-216763, BIO (6-bromoindirubin-3′-oxime), LY2090314, DCA, 2-amino-4-[3,4-(methylenedioxy)benzyl-amino]-6-(3-methoxyphenyl)pyrimidine, (hetero)arylpyrimidines, derivatives thereof, and combinations thereof. In certain embodiments, the one or more Wnt activator comprises CHIR99021.

In certain embodiments, the one or more GH inducer is selected from the group consisting of retinoic acid (RA), corticosteroids, thyroid hormones, ER agonists, GHRH signaling pathway agonists, Ghrelin signaling pathway agonists, interleukins, derivatives thereof, and mixtures thereof.

In certain embodiments, the corticosteroid is selected from the group consisting of dexamethasone, cortisone, hydrocortisone, derivatives thereof, and mixtures thereof. In certain embodiments, the corticosteroid comprises dexamethasone.

In certain embodiments, the thyroid hormone is selected from the group consisting of T3, T4, derivatives thereof, and mixtures thereof. In certain embodiments, the thyroid hormone comprises T3.

In certain embodiments, the GHRH signaling pathway agonist is selected from the group consisting of GHRH, c-AMP (e.g., Dibutyryl-cAMP), PKA, CREB, MAPK activator, derivatives thereof, and mixtures thereof. In certain embodiments, the GHRH signaling pathway agonist is selected from the group consisting of GHRH, c-AMP, and combinations thereof.

In certain embodiments, the Ghrelin signaling pathway agonist is selected from the group consisting of Ghrelin, GHSR agonists, derivatives thereof and mixtures thereof.

In certain embodiments, the interleukin is selected from the group consisting of IL-1, IL-2, IL-3, IL-4, IL-5, IL-6, IL-7, IL-8, IL-9, IL-10, IL-11, IL-12, IL-13, IL-15, and combinations thereof. In certain embodiments, the interleukin is selected from the group consisting of IL-1, IL-6, IL-10, and combinations thereof. In certain embodiments, the interleukin is IL-6.

In certain embodiments, the ER agonist is selected from the group consisting of diarylpropionitrile (DPN), Estradiol (E2), propylpyrazole-triol (PPT), derivatives thereof, and mixtures thereof. In certain embodiments, the ER agonist comprises DPN.

In certain embodiments, the one or more inhibitor of TGFβ/Activin-Nodal signaling is selected from the group consisting of SB431542, derivatives thereof, and mixtures thereof. In certain embodiments, the one or more inhibitor of TGFβ/Activin-Nodal signaling comprises SB431542.

In certain embodiments, the one or more activator of SHH signaling is selected from the group consisting of SHH, C25II and smoothened (SMO) receptor small molecule agonists such as purmorphamine, SAG (for example, as disclosed in Stanton et al, Mol Biosyst. 2010 January; 6 (1): 44-54), derivatives thereof, and mixtures thereof. In certain embodiments, the one or more activator of SHH signaling comprises SHH. In certain embodiments, the one or more activator of SHH signaling comprises SAG.

In certain embodiments, the one or more inhibitor of SMAD signaling is selected from the group consisting of LDN193189, Noggin, Dorsomorphin, K02288, DMH1, ML347, LDN 212854, derivatives thereof, and mixtures thereof. In certain embodiments, the one or more inhibitor of SMAD signaling comprises LDN193189.

In certain embodiments, the cells are contacted with the one or more dorsalizing agent at a concentration of between about 10 ng/ml and 200 ng/mL.

In certain embodiments, the cells are contacted with the one or more ventralizing agent at a concentration of between about 1 ng/ml and 30 ng/mL.

In certain embodiments, the cells are contacted with the one or more activator of FGF signaling at a concentration of between about 10 ng/ml and 200 ng/mL.

In certain embodiments, the cells are contacted with the one or more activator of BMP molecule at a concentration of between about 0.1 ng/ml and 10 ng/ml.

In certain embodiments, the cells are contacted with the one or more inhibitor of TGFβ/Activin-Nodal signaling at a concentration of between about 1 μM and 20 μM.

In certain embodiments, the cells are contacted with the one or more activator of SHH signaling at a concentration of between about 50 ng/ml and 200 ng/ml or of between about 50 nM and 200 nM.

In certain embodiments, cells are contacted with the one or more Wnt activator agent at a concentration of from about 1 μM to 10 μM.

In certain embodiments, cells are contacted with the one or more ER agonist at a concentration of between about 0.1 nM and 20 nM.

In certain embodiments, the cells are contacted with RA at a concentration of from about 0.1 μM to 1 μM.

In certain embodiments, the cells are contacted with one or more thyroid hormone at a concentration of between about 1 nM and 20 nM.

In certain embodiments, the cells are contacted with one or more corticosteroid at a concentration of from about 0.1 μM to 10 μM.

In certain embodiments, the cells are contacted with one or more GHRH signaling pathway agonist at a concentration of from about 0.1 μM to 10 UM, less than about 0.1 μM, or about 10 nM.

In certain embodiments, the cells are contacted with one or more Ghrelin signaling pathway agonist at a concentration of between about 1 nM and 50 nM.

In certain embodiments, the cells are contacted with one or more interleukins at a concentration of between about 1 ng/ml and 50 ng/ml.

In certain embodiments, the cells are contacted with the one or more activator of SHH signaling at a concentration of between about 50 nM and 200 nM.

In certain embodiments, the cells are contacted with the one or more inhibitor of SMAD signaling at a concentration of between about 100 nM and 500 nM.

Furthermore, the presently disclosed subject matter provides for kits for inducing differentiation of stem cells.

In certain embodiments, a kit for inducing differentiation of stem cells to a population of differentiated cells that express one or more pituitary precursor marker comprises: (a) one or more inhibitor of transforming growth factor beta (TGFβ)/Activin-Nodal signaling, (b) one or more activator of BMP signaling, (c) one or more activator of FGF signaling, and (d) one or more activator of SHH signaling. In certain embodiments, the kit further comprises (e) instructions for inducing differentiation of the stem cells into a population of differentiated cells that express one or more pituitary precursor marker. In certain embodiments, the kit further comprises (f) one or more SMAD inhibitor.

In certain embodiments, a kit for inducing differentiation of stem cells to a population of differentiated cells that express Pit1 comprises: (a) one or more inhibitor of transforming growth factor beta (TGFβ)/Activin-Nodal signaling, (b) one or more activator of BMP signaling, (c) one or more activator of FGF signaling, (d) one or more activator of SHH signaling, (e) one or more dorsalizing agent, (f) one or more ventralizing agent, and (g) one or more Wnt activator. In certain embodiments, the kit further comprises (h) instructions for inducing differentiation of the stem cells into a population of differentiated cells that express Pit1. In certain embodiments, the kit further comprises (i) one or more ER agonist.

In certain embodiments, a kit for inducing differentiation of stem cells to GH-producing somatotrophs comprises: (a) one or more inhibitor of transforming growth factor beta (TGFβ)/Activin-Nodal signaling, (b) one or more activator of BMP signaling, (c) one or more activator of FGF signaling, (d) one or more activator of SHH signaling, (e) one or more dorsalizing agent, (f) one or more ventralizing agent, (g) one or more Wnt activator, and (h) one or more GH inducer. In certain embodiments, the kit further comprises (i) instructions for inducing differentiation of the stem cells into a population of GH-producing somatotrophs.

The present disclosure further provides kits for inducing differentiation of pituitary precursors to GH-producing somatotrophs. In certain embodiments, the kit comprises: (a) one or more dorsalizing agent; (b) one or more ventralizing agent; (c) one or more activator of Wingless (Wnt) signaling (Wnt activator); and (d) one or more growth hormone (GH) inducer.

In addition, the present disclosure provides kits for inducing differentiation of stem cells to GH-producing somatotrophs. In certain embodiments, the kit comprises: (a) one or more BMP molecule; (b) one or more inhibitor of TGFβ/Activin-Nodal signaling; (c) one or more activator of FGF signaling; (d) one or more activator of SHH signaling; (e) one or more dorsalizing agent; (f) one or more ventralizing agent; (g) one or more activator of Wingless (Wnt) signaling (Wnt activator); and (h) one or more growth hormone (GH) inducer. In certain embodiment, the kit further comprises one or more ER agonist.

In certain embodiments, the one or more inhibitor of TGFβ/Activin-Nodal signaling is a small molecule selected from the group consisting of SB431542, derivatives thereof, and mixtures thereof. In certain embodiments, the inhibitor of TGFβ/Activin-Nodal signaling comprises SB431542.

In certain embodiments, said one or more inhibitor of SMAD signaling is a small molecule selected from the group consisting of LDN193189, Noggin, Dorsomorphin, K02288, DMH1, ML347, LDN 212854, derivatives thereof, and mixtures thereof. In certain embodiments, the inhibitor of SMAD signaling comprises LDN193189.

In certain embodiments, said one or more activator of Wnt signaling lowers glycogen synthase kinase 3B (GSK3B) for activation of Wnt signaling. In certain embodiments, said one or more activator of Wnt signaling is a small molecule selected from the group consisting of CHIR99021, Wnt-1, WNT3A, Wnt4, Wnt5a, derivatives thereof, and mixtures thereof. In certain embodiments, the activator of Wnt signaling comprises CHIR99021.

In certain embodiments, said activators of FGF signaling are selected from the group consisting of FGF1, FGF2, FGF3, FGF4, FGF7, FGF8, FGF10, FGF18, derivatives thereof, and mixtures thereof. In certain embodiments, the FGF activators comprise FGF8, FGF10, and/or FGF18.

In certain embodiments, said activator of BMP signaling is selected from the group consisting of BMP1, BMP2, BMP3, BMP4, BMP5, BMP6, BMP7, BMP8a, BMP8b, BMP10, BMP11, BMP15, derivatives thereof, and mixtures thereof.

In certain embodiments, the activator of SHH signaling is selected from the group consisting of Sonic hedgehog (SHH), C25II and smoothened (SMO) receptor small molecule agonists such as purmorphamine, derivatives thereof, and mixtures thereof.

In certain embodiments, the ER agonist is selected from the group consisting of diarylpropionitrile (DPN), Estradiol (E2), propylpyrazole-triol (PPT), derivatives thereof, and mixtures thereof. In certain embodiments, the ER agonist comprises DPN.

In certain embodiments, the GH inducer is selected from the group consisting of acid (RA), corticosteroids, thyroid hormones, ER agonists, and GHRH signaling pathway agonists, derivatives thereof, and mixtures thereof. In certain embodiments, the corticosteroid is selected from the group consisting of dexamethasone, cortisone, and hydrocortisone, derivatives thereof, and mixtures thereof. In certain embodiments, the thyroid hormones is selected from the group consisting of T3, T4, derivatives thereof, and mixtures thereof. In certain embodiments, the GHRH signaling pathway agonist is selected from the group consisting of GHRH, Dibutyryl-cAMP, PKA, CREB, MAPK activator, derivatives thereof, and mixtures thereof.

Additionally, the present disclosure provides various cell populations of differentiated cells obtained by the methods disclosed herein, including cell populations of in vitro differentiated GH-producing somatotrophs, cell populations of in vitro differentiated Pit1⁺ cells.

The present disclosure further provides compositions comprising the cell populations disclosed herein.

The present disclosure further provides a composition comprising a population of in vitro differentiated cells, wherein at least about 50% of differentiated cells are capable of producing growth hormone (GH-producing somatotrophs), and wherein less than about 25% of differentiated cells express one or more marker selected from the group consisting of lactrotroph markers, thyrotroph markers, pituitary precursor markers, Pit1, stem cell markers, NNE markers, neural crest (NC) lineage markers, and non-pituitary placode markers.

In certain embodiments, the presently disclosed subject matter provides for a population of in vitro differentiated cells expressing one or more somatotroph marker, wherein said differentiated cell population is derived from a population of stem cells according to a method comprising exposing a population of stem cells to effective amounts of (a) an effective amount of one or more inhibitor of transforming growth factor beta (TGFβ)/Activin-Nodal signaling, (b) an effective amount of one or more activator of BMP signaling, (c) an effective amount of one or more activator of Sonic Hedgehog (SHH) signaling, (d) an effective amount of two or more activators of FGF signaling, (e) an effective amount of one or more dorsalizing agent, (f) an effective amount of one or more ventralizing agent, and (g) an effective amount of one or more Wnt activator, and
wherein less than 25% of the population of differentiated cells express a detectable level of one or more marker selected from the group consisting of lactrotroph markers, thyrotroph markers, pituitary precursor markers, Pit1, stem cell markers, NNE markers, neural crest (NC) lineage markers, and non-pituitary placode markers (including, but not limited to, cranial placode markers, epibranchial placode markers, trigeminal placode markers, and otic placode markers.

The presently disclosed subject matter also provides a composition comprising a population of in vitro differentiated cells, wherein at least about 50% (e.g., at least about 55%, at least about 60%, at least about 65%, at least about 70%, at least about 75%, at least about 80%, at least about 85%, at least about 90%, at least about 95%, at least about 99%, or at least about 99.5%) of the population of cells express one or more somatotroph marker, and wherein less than about 25% (e.g., less than about 20%, less than about 15%, less than about 10%, less than about 5%, less than about 4%, less than about 3%, less than about 2%, less than about 1%, less than about 0.5%, or less than about 0.1%) of the population of cells express one or more marker selected from the group consisting of lactrotroph markers, thyrotroph markers, pituitary precursor markers, Pit1, stem cell markers, NNE markers, neural crest (NC) lineage markers, and non-pituitary placode markers (including, but not limited to, cranial placode markers, epibranchial placode markers, trigeminal placode markers, and otic placode markers).

In certain embodiments, the pituitary progenitor marker is selected from the group consisting of SIX1, LHX3, LHX4, PITX1, PITX2, HESX1, PROP1, SIX6, TBX19, and PAX6, GATA2, and SF1.

In certain embodiments, the somatotroph marker is selected from the group consisting of GH1, GHRH receptor (GHRHR), POU1F1, NeuroD4, and GHSR.

In certain embodiments, the NC lineage marker is selected from the group consisting of SOX10, FoxD3, ASCL1, Neurogenin, and Snail.

In certain embodiments, the NNE marker is selected from the group consisting of TFAP2A, EYA1, DLX3, and DLX5.

In certain embodiments, the non-pituitary placode marker is selected from the group consisting of cranial placode markers, epibranchial placode markers, trigeminal placode markers, and otic placode markers.

In certain embodiments, the cranial placode marker is selected from the group consisting of SIX1, PAX6, PITX3, Crystallin alpha A, and crystallin alpha B.

In certain embodiments, the trigeminal placode marker is PAX3.

In certain embodiments, the epibranchial placode marker is PAX2.

In certain embodiments, the otic placode marker is PAX8.

In certain embodiments, the lactrotroph marker is selected from the group consisting of PRL, PIT1, and D2R.

In certain embodiments, the thyrotroph marker is selected from the group consisting of TSH, THRH, and PIT1.

In certain non-limiting embodiments, the composition comprises a population of from about 1×10⁴ to about 1×10¹⁰ cells expressing said one or more somatotroph marker.

In certain embodiments, the stem cells are human stem cells. In certain embodiments, the human stem cells are human pluripotent stem cells. In certain embodiments, the human pluripotent stem cells are selected from the group consisting of human embryonic stem cells, and human induced pluripotent stem cells. In certain embodiments, the stem cells are non-human stem cells, for example, but not limited to, mammalian stem cells, primate stem cells, or stem cells from a rodent, a mouse, a rat, a dog, a cat, a horse, a pig, a cow, a sheep, etc.

The presently disclosed subject matter further provides methods of increasing growth hormone expression and/or secretion in a subject. In certain embodiments, the method comprises administering to the subject an effective amount of the differentiated cell population described herein (e.g., the cell population comprising at least about 50% (e.g., about 70% or about 80% of cells expressing one or more somatotroph marker) or a composition comprising thereof.

The presently disclosed subject matter further provides for a differentiated cell population described herein (e.g., the cell population comprising at least about 50% (e.g., about 70% or about 80% of cells expressing one or more somatotroph marker) a composition comprising thereof for increasing growth hormone expression and/or secretion in a subject.

The presently disclosed subject matter further provides methods of restoring dynamic release of one or more of GH, Insulin-like growth factor 1 (IGF-1), and IGF-2 in a subject. In certain embodiments, the method comprises administering to the subject an effective amount of the differentiated cell population described herein (e.g., the cell population comprising at least about 50% (e.g., about 70% or about 80% of cells expressing one or more somatotroph marker) or a composition comprising thereof.

The presently disclosed subject matter further provides a differentiated cell population described herein (e.g., the cell population comprising at least about 50% (e.g., about 70% or about 80% of cells expressing one or more somatotroph marker) a composition comprising thereof for restoring dynamic release of one or more of GH, Insulin-like growth factor 1 (IGF-1), and IGF-2 in a subject.

In certain embodiments, the subject suffers from growth hormone deficiency.

The presently disclosed subject matter further provides methods of treating growth hormone deficiency in a subject. In certain embodiments, the method comprises administering to the subject an effective amount of the differentiated cell population described herein (e.g., the cell population comprising at least about 50% (e.g., about 70% or about 80% of cells expressing one or more somatotroph marker) or a composition comprising thereof.

The present disclosure also provides methods of increasing GH expression and/or secretion, restoring dynamic release of one or more of GH, Insulin-like growth factor 1 (IGF-1), and IGF-2, and/or treating GH deficiency in a subject. In certain embodiments, the method comprises administering to the subject one of the followings: (a) a cell population of differentiated cells disclosed herein (e.g., a cell population of in vitro differentiated GH-producing somatotrophs disclosed herein); and (b) a composition disclosed herein.

The presently disclosed subject matter further provides a differentiated cell population described herein (e.g., the cell population comprising at least about 50% (e.g., about 70% or about 80% of cells expressing one or more somatotroph marker) a composition comprising thereof for treating growth hormone deficiency in a subject.

The presently disclosed subject matter further provides uses of the differentiated cell population described herein (e.g., the cell population comprising at least about 50% (e.g., about 70% or about 80% of cells expressing one or more somatotroph marker) a composition comprising thereof in the manufacture of a medicament for increasing growth hormone expression and/or secretion, restoring dynamic release of one or more of GH, Insulin-like growth factor 1 (IGF-1), and IGF-2, and/or treating growth hormone deficiency.

In addition, the present disclosure provides methods screening a therapeutic compound that is capable of overcoming one or more cellular phenotype that is related to GH deficiency. In certain embodiments, the method comprises: (a) contacting a cell population of in vitro differentiated GH-producing somatotrophs disclosed herein with a test compound; and (b) measuring functional activity and/or gene expression of the GH-producing somatotrophs, wherein the GH-producing somatotrophs are obtained from stem cells of a subject with GH deficiency or from cells expressing one or more pituitary precursor marker of a subject with GH deficiency.

### 4. BRIEF DESCRIPTION OF THE FIGURES

FIG. 1 depicts a method for differentiation of stem cells into somatotrophs in accordance with certain embodiments of the presently disclosed subject matter.

FIG. 2 depicts gene expressions of pre-placodal ectoderm and anterior pituitary progenitors derived from hPSCs. Single cell RNA-seq analysis showed that 70% of cells co-expressing pituitary transcripts such as PITX1 and LHX3. Only four cells (5% of all cells analyzed) expressed T (mesoderm), SOX17, or MYOD, suggesting a low percentage of contaminating cells. The other placode fates included PAX2 (epibranchial), PAX3 (trigeminal), or PAX8 (otic), which together were detected in about 20% of the SIX1+ population.

FIG. 3 depicts gene expression of pluripotent stem cells treated with low dose and high dose of Notch signaling inducers (e.g., FGF8/10). PROP1 expression was significantly upregulated by the Notch signaling inducers.

FIG. 4 depicts Pit1 gene expression in cells further treated by CHIR99021. Most Pit1 expressing cells were not proliferating shown by Ki67 labeling.

FIG. 5 depicts the effects of FGF8 on PROP1, PIT1 and POMC expression. FGF8 treatment maintained PROP1, downregulated PIT1 and upregulated POMC. BMP2 treatment neutralized such effects.

FIG. 6 depicts the effects of the combination of BMP2 and CHIR on GH1, PRL and TSH-β expression. The combination of BMP2 and CHIR significantly promoted GH1 expression. **: p-value<0.01 compared with the control group.

FIGS. 7A-7D depict the effects of RA, GHRH, T3, and corticosterone on selective induction of growth hormone secreting cells. FIG. 7A shows that treatment with RA significantly increased GH1 expression. FIG. 7B shows that treatment with GHRH significantly increased GH1 expression. FIG. 7C shows that treatment with T3 significantly reduced TSH-β expression. FIG. 7D shows that treatment with corticosterone significantly reduced PRL expression. **: p-value<0.01 compared with the control groups.

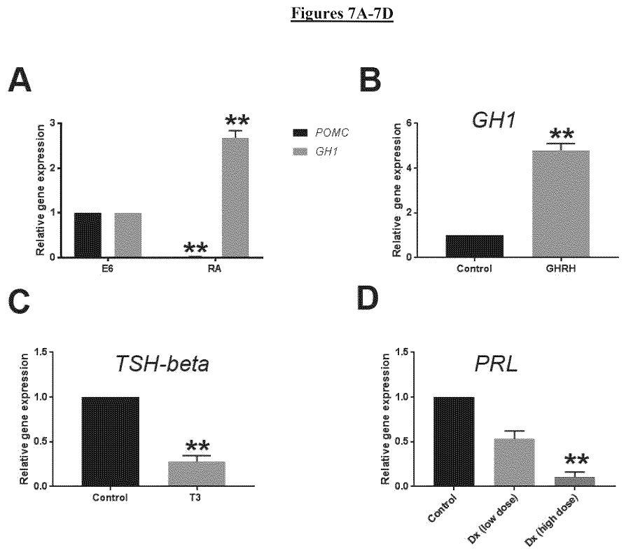

FIG. 8 depicts cell phenotype after growth hormone induction. The cell population expressed high amount of GH and GHRHR, and low amount of TSH, PRL and LH. The lower right panel shows that GH expression was increased upon GHRH stimulation.

FIG. 9 depicts the expressions of Ki67 and GHRHR in GHRHR positive cell population. In GHRHR positive cells, most cells expressed low level of GHRHR and only some of the cells expressed high level of GHRHR. The cells expressing high level of GHRHR exhibited polygonal morphology with lower proliferating rate, indicating they were more mature GH cells.

FIG. 10 depicts the correlation between growth hormone secretion and GHRHR expression. The left panel shows that cells expressing high level of GHRHR had low proliferating rate reflected by the low Ki67 expression. The middle panel shows that cells were screened based on GHRHR expression where cells expressing high level of GHRHR accounted for 15% of the population. The right panel shows that cells expressing high level of GHRHR expressed significantly higher amount of growth hormone compared to cells expressing low level of GHRHR. ***: p-value<0.001 compared with the control group.

FIG. 11 depicts an exemplary reporter construct for generating stage specific reporter lines whereby distinct cell population can be analyzed.

FIGS. 12A-12F depict the characterization of the Ames dwarf mouse colony. FIG. 12A depicts the comparison of Ames dwarf mice and heterozygous littermates at 11 weeks of age. The Ames dwarf mouse was diminutive in size and body weight. FIG. 12B depicts circulating plasma GH level as detected by ELISA. FIG. 12C depicts RT-qPCR analysis of IGF-1 (class II) gene expression in the gastrocnemius muscle. FIG. 12D depicts femurs obtained from the Ames mouse and a normal control exhibited a significant difference in length. E) MicroCT analysis of cortical bone density and thickness in the femur. FIG. 12F depicts MicroCT analysis of trabecular bone mineral density (TMD). TMD was similar among the 2 mice but Ames mice showed a much thinner trabecular bone that showed more separation and a higher trabecular number. Data were plotted as mean±SEM of two to four independent samples. *p<0.05, **: p-value<0.01, ***: p-value<0.001 compared with the wild type group.

FIGS. 13A-13F depict the effect of GH cells on RAG1^{-/-}/Prop1^{df/df} mice. FIG. 13A depicts the left two mice were RAG1^{-/-}/Prop1^{df/df}. The right mouse was RAG1^{-/-}/Prop1^{wt/df}. The mice were 6 months old. FIG. 13B depicts IHC that shows the presence of GH cells in human cells labeled with human nuclei (hNA) within the graft 6 weeks post graft. FIG. 13C shows that Basal and GHRH stimulated hGH plasma level detected by ELISA. FIG. 13D depicts qRT-PCR analysis of IGF-1 class II (circulating form of IGF-1). FIG. 13E depicts qRT-PCR analysis of IGFBP3 mRNA expression in liver. FIG. 13F depicts qRT-PCR analysis of basal plasma IGF-1 level detected by ELISA. n=2-3. Data were plotted as mean±SEM. Scale bar=50 μm. *: p-value<0.05; **: p-value<0.01 compared with the sham group.

FIG. 14 depicts a method for differentiation of stem cells into somatotrophs in accordance with certain embodiments of the presently disclosed subject matter.

FIG. 15 depicts a method for differentiation of stem cells into somatotrophs in accordance with certain embodiments of the presently disclosed subject matter.

FIG. 16 depicts interleukins (“ILs”) promote the gene expression of GH1. IL 6 and IL10 increased the gene expression of PIT1 and GH1 compared with the control group. **: p-value<0.001 compared with the control group.

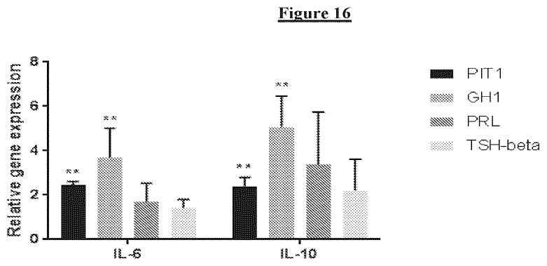

### 5. DETAILED DESCRIPTION OF THE INVENTION

The presently disclosed subject matter relates to in vitro methods for inducing differentiation of stem cells (e.g., human stem cells) to cells that express one or more somatotroph marker or to GH-producing somatotrophs, and in vitro methods for inducing differentiation of pituitary progenitors (or pituitary precursors) to cells that express one or more somatotroph marker or to GH-producing somatotrophs, and cells produced by such methods, and compositions comprising such cells. Also provided are uses of such cells for increasing growth hormone expression and/secretion, resorting dynamic release of growth hormone, and/or treating growth hormone deficiency.

For purposes of clarity of disclosure and not by way of limitation, the detailed description is divided into the following subsections:
5.1. Definitions;
5.2. Method of Differentiating Stem Cells;
5.3 Compositions Comprising Differentiated Cell Populations;
5.4. Method of Increasing Growth Hormone;
5.5. Kits;
5.6 Methods of Screening Therapeutic Compounds

### 5.1 Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “signaling” in reference to a “signal transduction protein” refers to a protein that is activated or otherwise affected by ligand binding to a membrane receptor protein or some other stimulus. Examples of signal transduction protein include, but are not limited to, a SMAD, a wingless (Wnt) complex protein, including beta-catenin, NOTCH, transforming growth factor beta (TGFβ), Activin, Nodal, glycogen synthase kinase 3β (GSK3 β) proteins, bone morphogenetic proteins (BMP), fibroblast growth factors (FGF), Sonic Hedgehog (SHH), GHRH, and Ghrelin. For many cell surface receptors or internal receptor proteins, ligand-receptor interactions are not directly linked to the cell's response. The ligand activated receptor can first interact with other proteins inside the cell before the ultimate physiological effect of the ligand on the cell's behavior is produced. Often, the behavior of a chain of several interacting cell proteins is altered following receptor activation or inhibition. The entire set of cell changes induced by receptor activation is called a signal transduction mechanism or signaling pathway.

As used herein, the term “signals” refer to internal and external factors that control changes in cell structure and function. They can be chemical or physical in nature.

As used herein, the term “ligands” refers to molecules and proteins that bind to receptors, e.g., transforming growth factor-beta (TFGβ), Activin, Nodal, bone morphogenic proteins (BMPs), etc.

“Inhibitor” as used herein, refers to a compound or molecule (e.g., small molecule, peptide, peptidomimetic, natural compound, siRNA, anti-sense nucleic acid, aptamer, or antibody) that is capable of interfering with (e.g., reduces, decreases, suppresses, eliminates, or blocks) the signaling function of s molecule or pathway. An inhibitor can be any compound or molecule that changes any activity of a named protein (signaling molecule, any molecule involved with the named signaling molecule, a named associated molecule). Inhibitors are described in terms of competitive inhibition (binds to the active site in a manner as to exclude or reduce the binding of another known binding compound) and allosteric inhibition (binds to a protein in a manner to change the protein conformation in a manner which interferes with binding of a compound to that protein's active site) in addition to inhibition induced by binding to and affecting a molecule upstream from the named signaling molecule that in turn causes inhibition of the named molecule. An inhibitor can be a “direct inhibitor” that inhibits a signaling target or a signaling target pathway by actually contacting the signaling target.

“Activator,” as used herein, refers to a compound that is capable of increasing, inducing, stimulating, activating, facilitating, and/or enhancing activation the signaling function of the molecule or pathway, e.g., Wnt signaling, BMP signaling, SHH signaling, FGF signaling, GHRH signaling, Ghrelin signaling, etc.

As used herein, the term “derivative” refers to a chemical compound with a similar core structure.

As used herein, the term “a population of cells” or “a cell population” refers to a group of at least two cells. In non-limiting examples, a cell population can include at least about 10, at least about 100, at least about 200, at least about 300, at least about 400, at least about 500, at least about 600, at least about 700, at least about 800, at least about 900, at least about 1000 cells, at least about 5,000 cells or at least about 10,000 cells or at least about 100,000 cells or at least about 1,000,000 cells. The population may be a pure population comprising one cell type, such as a population of pituitary progenitors or pituitary precursors, cells expressing Pit1, GH-producing somatotrophs (GHRHR^{low} cells, GHRHR^{high} cells), or a population of undifferentiated stem cells. Alternatively, the population may comprise more than one cell type, for example a mixed cell population.

As used herein, the term “stem cell” refers to a cell with the ability to divide for indefinite periods in culture and to give rise to specialized cells. A human stem cell refers to a stem cell that is from a human.

As used herein, the term “embryonic stem cell” and “ESC” refer to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “embryonic stem cell line” refers to a population of embryonic stem cells which have been cultured under in vitro conditions that allow proliferation without differentiation for up to days, months to years.

As used herein, the term “totipotent” refers to an ability to give rise to all the cell types of the body plus all of the cell types that make up the extraembryonic tissues such as the placenta.

As used herein, the term “multipotent” refers to an ability to develop into more than one cell type of the body.

As used herein, the term “pluripotent” refers to an ability to develop into the three developmental germ layers of the organism including endoderm, mesoderm, and ectoderm.

As used herein, the term “induced pluripotent stem cell” or “iPSC” refers to a type of pluripotent stem cell, similar to an embryonic stem cell, formed by the introduction of certain embryonic genes (such as a OCT4, SOX2, and KLF4 transgenes) (see, for example, Takahashi and Yamanaka Cell 126, 663-676 (2006), herein incorporated by reference) into a somatic cell, for examples, CI 4, C72, and the like.

As used herein, the term “somatic cell” refers to any cell in the body other than gametes (egg or sperm); sometimes referred to as “adult” cells.

As used herein, the term “somatic (adult) stem cell” refers to a relatively rare undifferentiated cell found in many organs and differentiated tissues with a limited capacity for both self renewal (in the laboratory) and differentiation. Such cells vary in their differentiation capacity, but it is usually limited to cell types in the organ of origin.

As used herein, the term “neuron” refers to a nerve cell, the principal functional units of the nervous system. A neuron consists of a cell body and its processes—an axon and one or more dendrites. Neurons transmit information to other neurons or cells by releasing neurotransmitters at synapses.

As used herein, the term “proliferation” refers to an increase in cell number.

As used herein, the term “undifferentiated” refers to a cell that has not yet developed into a specialized cell type.

As used herein, the term “differentiation” refers to a process whereby an unspecialized embryonic cell acquires the features of a specialized cell such as a heart, liver, or muscle cell. Differentiation is controlled by the interaction of a cell's genes with the physical and chemical conditions outside the cell, usually through signaling pathways involving proteins embedded in the cell surface.

As used herein, the term “directed differentiation” refers to a manipulation of stem cell culture conditions to induce differentiation into a particular (for example, desired) cell type, such as neural, neural crest, cranial placode, and non-neural ectoderm precursors.

As used herein, the term “directed differentiation” in reference to a stem cell refers to the use of small molecules, growth factor proteins, and other growth conditions to promote the transition of a stem cell from the pluripotent state into a more mature or specialized cell fate.

As used herein, the term “pituitary progenitor” and “pituitary precursor” are used interchangeably, referring to a cell expressing one or more pituitary progenitor marker.

As used herein, the term “somatotroph” refers to a cell expressing one or more somatotroph marker. In certain embodiments, the somatotrophs are GH-producing somatotrophs or GH-secreting somatotrophs.

As used herein, the term “inducing differentiation” in reference to a cell refers to changing the default cell type (genotype and/or phenotype) to a non-default cell type (genotype and/or phenotype). Thus, “inducing differentiation in a cell (e.g., a stem cell)” refers to inducing the cell (e.g., stem cell) to divide into progeny cells with characteristics that are different from that cell, such as genotype (e.g., change in gene expression as determined by genetic analysis such as a microarray) and/or phenotype (e.g., change in expression of a protein, such as a pituitary progenitor marker, PIT1, and a somatotroph marker).

As used herein, the term “cell culture” refers to a growth of cells in vitro in an artificial medium for research or medical treatment.

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

As used herein, the term “contacting” cells with a compound (e.g., one or more inhibitor, activator, and/or inducer) refers to exposing cells to a compound, for example, placing the compound in a location that will allow it to touch the cell. The contacting may be accomplished using any suitable methods. For example, contacting can be accomplished by adding the compound to a tube of cells. Contacting may also be accomplished by adding the compound to a culture medium comprising the cells. Each of the compounds (e.g., the inhibitors and activators disclosed herein) can be added to a culture medium comprising the cells as a solution (e.g., a concentrated solution). Alternatively or additionally, the compounds (e.g., the inhibitors and activators disclosed herein) as well as the cells can be present in a formulated cell culture medium.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

As used herein, the term “expressing” in relation to a gene or protein refers to making an mRNA or protein which can be observed using assays such as microarray assays, antibody staining assays, and the like.

As used herein, the term “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type.

As used herein, the term “derived from” or “established from” or “differentiated from” when made in reference to any cell disclosed herein refers to a cell that was obtained from (e.g., isolated, purified, etc.) a parent cell in a cell line, tissue (such as a dissociated embryo, or fluids using any manipulation, such as, without limitation, single cell isolation, culture in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

### 5.2 Method of Differentiating Stem Cells

The presently disclosed subject matter provides for in vitro methods for inducing differentiation of stem cells (e.g., human stem cells). In certain embodiments, the stem cell is a pluripotent stem cell. In certain embodiments, the stem cell is a human stem cell. In certain embodiments, the stem cell is a human pluripotent stem cell. Non-limiting examples of human stem cells include human embryonic stem cells (hESC), human pluripotent stem cell (hPSC), human induced pluripotent stem cells (hiPSC), human parthenogenetic stem cells, primordial germ cell-like pluripotent stem cells, epiblast stem cells, F-class pluripotent stem cells, somatic stem cells, cancer stem cells, or any other cell capable of lineage specific differentiation. In certain embodiments, the human stem cell is a human embryonic stem cell (hESC). In certain embodiments, the human stem cell is a human induced pluripotent stem cell (hiPSC). In certain embodiments, the stem cells are non-human stem cells, including, but not limited to, mammalian stem cells, primate stem cells, or stem cells from a rodent, a mouse, a rat, a dog, a cat, a horse, a pig, a cow, a sheep, etc.

In certain embodiments, the presently disclosed subject matter relates to methods of making in-vitro-stem-cell-derived somatotrophs (e.g., GH-producing somatotrophs). In certain embodiments, the in vitro differentiation of stem cells to somatotrophs include three phases: (a) in vitro differentiation of stem cells to pituitary progenitors, (b) in vitro differentiation of pituitary progenitors to cells expressing Pit1 (Pit1⁺ cells); and (c) in vitro differentiation of Pit1⁺ cells to GH-producing somatotrophs.

In certain embodiments, the presently disclosed subject matter relates to methods of inducing differentiation of pituitary progenitors to somatotrophs (e.g., GH-producing somatotrophs). In certain embodiments, the method comprises in vitro differentiation of pituitary progenitors to cells expressing Pit1 (Pit1⁺ cells); and in vitro differentiation of Pit1⁺ cells to GH-producing somatotrophs.

Any suitable methods for in vitro differentiation of stem cells to pituitary progenitors, including, but not limited to, those disclosed in Zimmer, et al., Stem Cell Report (2016); 6:858-872, and U.S. Patent Provisional application No. 62/555,629 filed Sep. 7, 2017; can be used in the first phase of the presently disclosed method.

In certain embodiments, a population of stem cells are in vitro differentiated to a population of pituitary progenitors, which are in vitro differentiated to a population of Pit1⁺ cells, which are further in vitro differentiated to a population of somatotrophs.

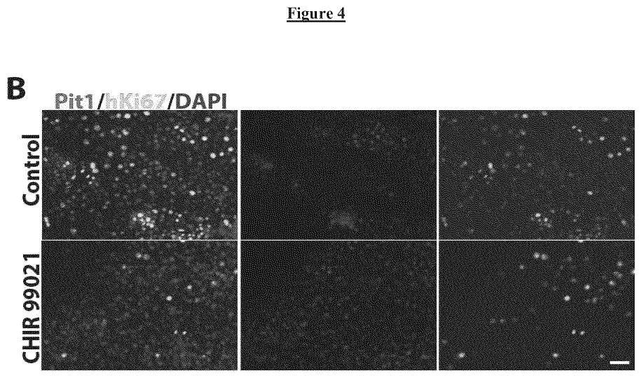

The presently disclosed method is at least based on the inventors' discoveries that an activator of Wnt signaling (referred to as “Wnt activator”; e.g., CHIR99021) can enhance Pit1 induction via upregulating Wnt/β-catenin signaling; an activator of FGF signaling (referred to as “FGF activator”, e.g., FGF8) can maintain Prop1 and downregulate Pit1 while upregulating POMC, which effect can be antagonized by a bone morphogenetic protein (BMP) molecule (e.g., BMP2); and the combination of an FGF activator, a BMP molecule and a Wnt activator (e.g., a combination of FGF8, BMP2 and CHIR) can promotes GH1 expression. See FIG. 4-6. Furthermore, Pit1⁺ cells can give rise to somatotrophs, lactrotrophs and thyrotrophs (Tabar et al., Cell Stem Cell. (2011 Dec. 2); 9 (6): 490-1). The inventors discovered the following molecules that are capable of inducing selective differentiation of Pit1⁺ cells to somatotrophs: retinoic acid (suppressing POMC expressed by corticotrophs and promote GH1 expression), corticosteroid (e.g. dexamethasone) and thyroid hormone (e.g., T3) (inhibiting PRL (expressed by lactotroph) and TSH (expressed by thyrotroph) while promoting GH1 expression), ER agonists (e.g., DPN) (promoting GH1 expression), GHRH signaling pathway agonists (e.g., GHRH, c-AMP) (promoting GH1 expression), Ghrelin signaling pathway agonists (e.g., Ghrelin) (promoting GH1 expression) and Interleukins (e.g., IL-6) (promoting GH1 expression).

5.2.1. Method of Differentiating Stem cells to Pituitary Precursors

In certain embodiments, the stem cells are differentiated into pituitary precursors, pituitary placode precursors or pituitary cells. In certain embodiments, the pituitary precursors are anterior pituitary precursors. In certain embodiments, the stem cells are differentiated into pituitary precursors, pituitary cells, or pituitary placode precursors, wherein the stem cells are contacted with an effective amount of one or more inhibitor of TGFβ/Activin-Nodal signaling and an effective amount of one or more activator of BMP signaling (BMP activator, e.g., a BMP molecule), and the cells are contacted with an effective amount of one or more activator of Sonic Hedgehog (SHH) signaling (SHH activator) and an effective amount of one, two or more activator of FGF signaling. In certain embodiments, the cells are further contacted with an effective amount of one or more inhibitor that is capable of inhibiting Small Mothers Against Decapentaplegic (SMAD) signaling (“SMAD inhibitor”).

In certain embodiments, the activator(s) of FGF signaling activates FGF8 and FGF10 signaling. In certain embodiments, the activator(s) of FGF signaling further activates the FGF18 signaling. Non-limiting examples of FGF activators include FGF1, FGF2, FGF3, FGF4, FGF7, FGF8, FGF10, FGF18, derivatives thereof, and mixtures thereof. In certain embodiments, the differentiation method includes contacting the cells with two FGF activators. In certain embodiments, the two FGF activators are FGF8 and FGF10. In certain embodiments, the differentiation method includes contacting the cells with three FGF activators. In certain embodiments, the three FGF activators are FGF8, FGF10, and FGF18.

In certain embodiments, the one or more inhibitor of TGFβ/Activin-Nodal signaling neutralizes the ligands including TGFβs, BMPs, Nodal, and activins, or blocking their signal pathways through blocking the receptors and downstream effectors. Non-limiting examples of inhibitors of TGFβ/Activin-Nodal signaling are disclosed in WO2011/149762, Chambers (2009), and Chambers (2012), which are incorporated by reference in their entireties. In certain embodiments, the one or more inhibitor of TGFβ/Activin-Nodal signaling is a small molecule selected from the group consisting of SB431542, derivatives thereof, and mixtures thereof. In certain embodiments, the one or more inhibitor of TGFβ/Activin-Nodal signaling is SB431542.

“SB431542” refers to a molecule with a number CAS 301836-41-9, a molecular formula of C₂₂H₁₈N₄O₃, and a name of 4-[4-(1,3-benzodioxol-5-yl)-5-(2-pyridinyl)-1H-imidazol-2-yl]-benzamide, for example, see structure below:

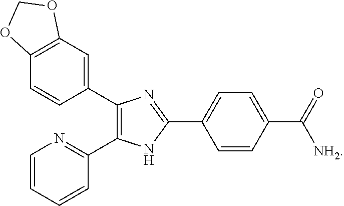 [SMILES: NC(=O)c1ccc(-c2nc(-c3ccc4c(c3)OCO4)c(-c3ccccn3)[nH]2)cc1]

Non-limiting examples of SMAD inhibitors are disclosed in WO2011/149762, Chambers (2009), and Chambers (2012), which are incorporated by reference in their entireties. Non-limiting examples of SMAD inhibitors include LDN193189, Noggin, Dorsomorphin, K02288, DMH1, ML347, LDN 212854, derivatives thereof, and mixtures thereof, and other BMP inhibitors. In certain embodiments, the one or more inhibitor of SMAD signaling is a small molecule selected from the group consisting of LDN193189, derivatives thereof, and mixtures thereof. In certain embodiments, the one or more SMAD inhibitor is LDN193189.

“LDN193189” refers to a small molecule DM-3189, IUPAC name 4-(6-(4-(piperazin-1-yl)phenyl)pyrazolo[1,5-a]pyrimidin-3-yl) quinoline, with a chemical formula of C₂₅H₂₂N₆ with the following formula.

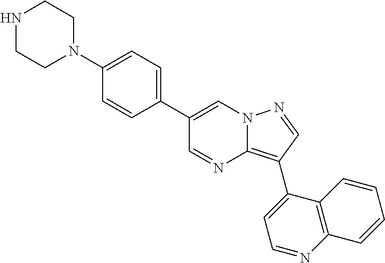 [SMILES: c1ccc2c(-c3cnn4cc(-c5ccc(N6CCNCC6)cc5)cnc34)ccnc2c1]

LDN193189 is capable of functioning as a SMAD signaling inhibitor. LDN193189 is also highly potent small-molecule inhibitor of ALK2, ALK3, and ALK6, protein tyrosine kinases (PTK), inhibiting signaling of members of the ALK1 and ALK3 families of type I TGFβ receptors, resulting in the inhibition of the transmission of multiple biological signals, including the bone morphogenetic proteins (BMP) BMP2, BMP4, BMP6, BMP7, and Activin cytokine signals and subsequently SMAD phosphorylation of Smad1, Smad5, and Smad8 (Yu et al. (2008) Nat Med 14:1363-1369; Cuny et al. (2008) Bioorg. Med. Chem. Lett. 18:4388-4392, herein incorporated by reference).

In certain embodiments, the BMP activator is a BMP molecule. Non-limiting examples of BMP activators include BMP1, BMP2, BMP3, BMP4, BMP5, BMP6, BMP7, BMP8a, BMP8b, BMP10, BMP11, BMP15, derivatives thereof, and mixtures thereof. In certain embodiments, the one or more BMP activator comprises BMP4.

In certain embodiments, the activator of SHH signaling is selected from the group consisting of Sonic hedgehog (SHH), C25II and smoothened (SMO) receptor small molecule agonists such as purmorphamine, Smoothened agonist (SAG) (for example, as disclosed in Stanton et al, Mol Biosyst. 2010 January; 6 (1): 44-54), derivatives thereof, and mixtures thereof. In certain embodiments, the one or more activator of SHH signaling comprises a SHH. In certain embodiments, the one or more activator of SHH signaling comprises a SAG.

In certain embodiments, the one or more inhibitor of TGFβ/Activin-Nodal signaling is contacted with (or exposed to) the stem cells for at least about 3 days, at least 4 about days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, or at least about 20 days to obtain pituitary progenitors. In certain embodiments, the one or more inhibitor of TGFβ/Activin-Nodal signaling is contacted with (or exposed to) the stem cells for up to about 3 days, up to about 4 days, up to about 5 days, up to about 6 days, up to about 7 days, up to about 8 days, up to about 9, days, up to about 10 days, up to about 11 days, up to about 12 days, up to about 13 days, up to about 14 days, up to about 15 days, up to about 16 days, up to about 17 days, up to about 18 days, up to about 19 days, or up to about 20 days to obtain pituitary progenitors. In certain embodiments, the cells are contacted with the one or more inhibitor of TGFβ/Activin-Nodal signaling for about 3 days to obtain pituitary progenitors. In certain embodiments, the cells are contacted with the one or more inhibitor of TGFβ/Activin-Nodal signaling for about 4 days to obtain pituitary progenitors. In certain embodiments, the cells are contacted with the one or more inhibitor of TGFβ/Activin-Nodal signaling for at least about 5 days to obtain pituitary progenitors. In certain embodiments, the cells are contacted with the inhibitor of TGFβ/Activin-Nodal signaling for about 8 days to obtain pituitary progenitors. In certain embodiments, the cells are contacted with the inhibitor of TGFβ/Activin-Nodal signaling for 9 days to obtain pituitary progenitors.

In certain embodiments, the one or more BMP activator is contacted with (or exposed to) the stem cells for at least about 2 days, at least about 3 days, at least about 4 days, or at least about 5 days, and/or for up to about 3 days, up to about 4 days, up to about 5 days, to obtain pituitary progenitors. In certain embodiments, the one or more BMP activator is contacted with (or exposed to) the stem cells for at least about 2 days and/or up to about 4 days to obtain pituitary progenitors. In certain embodiments, the one or more BMP activator is contacted with (or exposed to) the stem cells for about 3 days to obtain pituitary progenitors. In certain embodiments, the one or more BMP activator is contacted with (or exposed to) the stem cells for 4 days to obtain pituitary progenitors.

In certain embodiments, the one or more activator of SHH signaling and the one, two or more activators of FGF signaling, and optionally the one or more SMAD inhibitor are contacted with the cells for at least about 2 days, at least about 3 days, at least about 4 days, at least about 5 days, at least about 6 days, at least about 7 days, at least about 8 days, at least about 9 days, or at least about 10 days, to obtain pituitary progenitors. In certain embodiments, the cells are contacted with the one or more activator of SHH signaling and the one, two or more activators of FGF signaling, and optionally the one or more SMAD inhibitor for up to about 4 days, up to about 5 days, up to about 6 days, up to about 7 days, up to about 8 days, up to about 9 days, up to about 10 days, up to about 11 days, up to about 12 days, up to about 13 days, up to about 14 days, up to about 15 days, up to about 16 days, up to about 17 days, up to about 18 days, up to about 19 days, or up to about 20 days, to obtain pituitary progenitors. In certain embodiments, the one or more activator of SHH signaling and the one, two or more activators of FGF signaling, and optionally the one or more SMAD inhibitor are contacted to the cells for at least about 2 days, to obtain pituitary progenitors. In certain embodiments, the one or more activator of SHH signaling and the one, two or more activators of FGF signaling, and optionally the one or more SMAD inhibitor are contacted with the cells for at least about 5 days, to obtain pituitary progenitors. In certain embodiments, the one or more activator of SHH signaling and the one, two or more activators of FGF signaling, and optionally the one or more SMAD inhibitor are contacted with the cells for 6 days, to obtain pituitary progenitors.

In certain embodiments, the one or more activator of SHH and the one, two or more activators of FGF, and optionally the one or more SMAD inhibitor are initially contacted with (exposed to) the cells at least about 2 days, about 3 days, about 4 days, about 5 days, or about 6 days, and/or no later than about days, no later than about 4 days, no later than about 5 days, no later than about 6 days, no later than about 7 days, no later than about 8 days, no later than about 9 days, or no later than about 10 days from the initial contact of the stem cells with the one or more inhibitor of TGFβ/Activin-Nodal signaling, to obtain pituitary progenitors. In certain embodiments, one or more activator of SHH and one, two or more activators of FGF, and optionally the one or more SMAD inhibitor are initially contacted with (exposed to) the cells at least about 2 days and/or no later than about 5 days from the initial contact of the stem cells to the one or more inhibitor of TGFβ/Activin-Nodal signaling, to obtain pituitary progenitors. In certain embodiments, the one or more activator of SHH and the one, two or more activators of FGF, and optionally the one or more SMAD inhibitor are initially contacted with the cells about 3 days from the initial contact of the stem cells to the one or more inhibitor of TGFβ/Activin-Nodal signaling, to obtain pituitary progenitors. In certain embodiments, the one or more activator of SHH and the one, two or more activators of FGF, and optionally the one or more SMAD inhibitor are initially contacted with (exposed to) the cells 4 days from the initial contact of the stem cells to the one or more inhibitor of TGFβ/Activin-Nodal signaling, to obtain pituitary progenitors. In certain embodiments, the one or more activator of SHH, the one, two or more (e.g., three) FGF activators, and the one or more SMAD inhibitor are initially contacted with the cells about 3 days from the initial contact of the stem cells with the one or more inhibitor of TGFβ/Activin-Nodal signaling, to obtain pituitary progenitors.

In certain embodiments, the cells are contacted with (exposed to) the one or more inhibitor of TGFβ/Activin-Nodal signaling and the one or more BMP activator concurrently. In certain embodiments, the cells are contacted with (exposed to) the one or more activator of SHH and the one, two or more activators of FGF, and optionally the one or more SMAD inhibitor concurrently.

In certain embodiments, the cells are contacted with (exposed to) an inhibitor of TGFβ/Activin-Nodal signaling at a concentration of between about 1 μM and 20 μM, between about 2 μM and 18 μM, between about 4 and 16 μM, between about 6 UM and 14 μM, or between about 8 μM and 12 μM. In certain embodiments, the cells are contacted with an inhibitor of TGFβ/Activin-Nodal signaling at a concentration of about 10 μM. In certain embodiments, the cells are contacted to SB43152 at a concentration of about 10 μM.

In certain embodiments, the activator of BMP signaling or the BMP molecule is contacted with the cells at a concentration of between about 0.01 ng/ml and 10 ng/ml, between about 0.1 ng/ml and 8 ng/mL, between about 1 ng/ml and 10 ng/mL, between about 1 ng/ml and 6 ng/ml, between about 1 ng/ml and 5 ng/mL, or between about 2 ng/ml and 5 ng/mL. In certain embodiments, the cells are contacted with the activator of BMP signaling or the BMP molecule at a concentration of between about 1 ng/ml and 10 ng/mL. In certain embodiments, the cells are contacted with the activator of BMP signaling or the BMP molecule at a concentration of about 5 ng/mL. In certain embodiments, the cells are contacted to BMP4 at a concentration of about 5 ng/mL.

In certain embodiments, the cells are contacted with the two or more activators of FGF signaling, each at a concentration of between about 10 ng/ml and 200 ng/mL, between about 20 ng/ml and 150 ng/mL, between about 30 ng/ml and 100 ng/mL, or between about 40 ng/ml and 75 ng/mL, In certain embodiments, the cells are contacted with the one or more activator of FGF signaling at a concentration of about 50 ng/mL, or about 100 ng/mL. In certain embodiments, the cells are contacted with FGF8 in a concertation of about 100 ng/mL. In certain embodiments, the cells are contacted with FGF10 in a concertation of about 50 ng/mL. In certain embodiments, the cells are contacted with FGF18 in a concertation of about 50 ng/mL.

In certain embodiments, the cells are contacted with the one or more activator of SHH signaling at a concentration of between about 10 ng/ml and 400 ng/mL, between about 50 ng/ml and 350 ng/mL, between about 50 ng/ml and 250 ng/mL, between about 100 ng/ml and 300 ng/mL, between about 150 ng/ml and 250 ng/mL, or between about 50 ng/ml and 200 ng/ml. In certain embodiments, the cells are contacted with the one or more activator of SHH signaling at a concentration of between about 50 ng/ml and 200 ng/mL. In certain embodiments, the cells are contacted with the one or more activator of SHH signaling at a concentration of about 200 ng/mL. In certain embodiments, the cells are contacted with the one or more activator of SHH signaling at a concentration of about 100 ng/mL. In certain embodiments, the cells are contacted with SHH at a concentration of about 100 ng/mL.

In certain embodiments, the cells are contacted with the one or more activator of SHH signaling at a concentration of between about 10 nM and 400 nM, between about 50 nM and 350 nM, between about 50 nM and 250 nM, between about 100 nM and 300 nM, between about 50 nM and 200 nM, or between about 150 nM and 250 nM. In certain embodiments, the cells are contacted with the one or more activator of SHH signaling at a concentration of between about 50 nM and 200 nM. In certain embodiments, the cells are contacted with the one or more activator of SHH signaling at a concentration of about 200 nM. In certain embodiments, the cells are contacted with the one or more activator of SHH signaling at a concentration of about 100 nM. In certain embodiments, the cells are contacted with SAG at a concentration of about 100 nM.

In certain embodiments, the cells are contacted with the one or more SMAD inhibitor at a concentration of between of between about 10 nM and 300 nM, between about 10 nM and 100 nM, between about 10 nM and 50 nM, between about 20 nM and 50 nM, between about 20 nM and 40 nM, between about 10 nM and 30 nM, between about 30 nM and 50 nM, between about 50 nM and 100 nM, between about 50 nM and 60 nM, between about 50 nM and 80 nM, between about 60 nM and 80 nM, between about 100 nM and 200 nM, between about 100 nM and 150 nM, between about 150 nM and 200 nM, between about 200 nM and 300 nM, between about 200 nM and 250 nM, or between about 250 nM and 300 nM. In certain embodiments, the cells are contacted with the one or more SMAD inhibitor at a concentration of between about 10 nM and 30 nM. In certain embodiments, the cells are contacted with the one or more SMAD inhibitor at a concentration of about 25 nM. In certain embodiments, the cells are contacted with LDN 193189 at a concentration of about 25 nM. In certain embodiments, the cells are contacted with the one or more SMAD inhibitor at a concentration of between about 200 nM and 250 nM. In certain embodiments, the cells are contacted with the one or more SMAD inhibitor at a concentration of about 250 nM. In certain embodiments, the cells are contacted with LDN 193189 at a concentration of about 250 nM.

In certain embodiments, the cells are contacted with (or exposed to) each of the one or more inhibitor of TGFβ/Activin-Nodal signaling, the one or more BMP activator, the one or more SHH activator, the two or more FGF activators, and the one or more SMAD inhibitor daily. In certain embodiments, the cells are contacted with (or exposed to) each of the one or more inhibitor of TGFβ/Activin-Nodal signaling, the one or more BMP activator, the one or more SHH activator, the two or more FGF activators, and the one or more SMAD inhibitor every other day. In certain embodiments, the cells are contacted with (or exposed to) the one or more BMP activator daily. In certain embodiments, the cells are contacted with (or exposed to) the one or more inhibitor of TGFβ/Activin-Nodal signaling daily from day 0 to day 2, and every other day on day 3 and later. In certain embodiments, the cells are contacted with (or exposed to) the one or more SHH activator, the two or more FGF activators, and the one or more SMAD inhibitor every other day.

In certain embodiments, the differentiated cells express a detectable level of one or more pituitary progenitor marker. Non-limiting examples of pituitary progenitor markers include SIX1, LHX3, LHX4, PITX1, PITX2, HESX1, PROP1, SIX6, TBX19, and PAX6, GATA2, and SF1. In certain embodiments, the differentiated cells express a detectable level of one or more pituitary progenitor marker (e.g., an anterior pituitary progenitor marker) at least about 5 days, at least about 6 days, at least about 7 days, at least about 8 days, about 9 days, about 10 days, about 15 days, or about 30 days after the initial contact of the cells with the one or more inhibitor of TGFβ/Activin-Nodal signaling. In certain embodiments, the differentiated cells express a detectable level of one or more pituitary progenitor marker (e.g., an anterior pituitary progenitor marker) at least about 5 days from the initial contact of the cells with the one or more inhibitor of TGFβ/Activin-Nodal signaling. In certain embodiments, the differentiated cells express a detectable level of one or more pituitary progenitor marker (e.g., an anterior pituitary progenitor marker) about 8 days from the initial contact of the cells with the one or more inhibitor of TGFβ/Activin-Nodal signaling. In certain embodiments, greater than about 50%, about 60%, about 70%, about 80%, or about 90% of the population of cells (e.g., at about 8 days, about 9 days, about 10 days, about 15 days, or about 30 days from the initial contact of the cells with the one or more inhibitor of TGFβ/Activin-Nodal signaling) express a detectable level of the one or more pituitary progenitor marker (e.g., an anterior pituitary progenitor marker). In certain embodiments, greater than about 70% of the differentiated cells express a detectable level of the one or more pituitary progenitor marker (e.g., an anterior pituitary progenitor marker).

In certain embodiments, the method of inducing differentiation of the stem cells to pituitary cells, pituitary precursors or pituitary placode precursors comprises contacting the stem cells with the one or more inhibitor of TGFβ/Activin-Nodal signaling (e.g., 10 μM SB431542) and the one or more activator of BMP signaling (e.g., 5 ng/ml BMP4) (e.g., for about 3 days (e.g., 3 or 4 days)), and contacting the cells with the one or more activator of SHH signaling (e.g., 100 ng/mL SHH or 100 nM SAG), the two or more (e.g., three) activators of FGF signaling (e.g., 100 ng/mL FGF8.50 ng/ml FGF10, and 50 ng/mL FGF18), and the one or more SMAD inhibitor (e.g., 250 nM LDN 193189) (e.g., for about 5 days (e.g., 5 or 6 days)). In certain embodiments, the initial contact of the SHH activator(s), FGF activator(s) and SMAD inhibitor(s) with the cells is about 3 days (e.g., 3 or 4 days) from the initial contact of the stem cells with the one or more inhibitor of TGFβ/Activin-Nodal signaling, wherein the cells are contacted with (exposed to) the SHH activator(s), FGF activator(s) and SMAD inhibitor(s) for about 5 days (e.g., 5 or 6 days).

### 5.2.2. Method of Differentiating Pituitary Precursors to Pit1⁺ Cells

In certain embodiments, the pituitary precursors (e.g., the differentiated cells obtained from the method described in Section 5.2.1) are differentiated into cells expressing Pit1 (also known as “POU domain, class 1, transcription factor 1 (“Poulf1”)), namely “Pit1⁺ cells”. Pit1 is a pituitary-specific transcription factor responsible for pituitary development and hormone expression in mammals and is a member of the POU family of transcription factors that regulate mammalian development. During the mouse pituitary development, Pit1 was found to appear after the decreasing of the expression of Prop1, and the transition of Prop1 to Pit1 was found to be concomitant with the presence of FGF8 and BMP2 and Wnt/β-catening signaling which shown by the target factor Axin2 (Dasen J S et al., Annu Rev Neurosci. (2001); 24:327-55; Olson et al., Cell. (2006 May 5); 125 (3): 593-605). Wnt/β-Catenin signaling is required for Pit1 lineage determination, and Wnt/β-catenin/Prop1 complex is required to suppress HESX1 and activate Pit1 (Olson et al., 2006).

In certain embodiments, the presently disclosed subject matter provides methods of inducing differentiation of pituitary precursors to a first cell population comprising at least about 50%, at least about 60%, at least about 70%, at least about 80%, or at least about 90%, and up to about 100% Pit1⁺ cells. In certain embodiments, the method comprises contacting pituitary precursors with an effective amount of one or more dorsalizing agent and an effective amount of one or more ventralizing agent; and contacting the cells with an effective amount of one or more activator of Wnt signaling (referred to as “Wnt activator”). In certain embodiments, the method further comprises contacting the pituitary precursors with an effective amount of one or inhibitor of TGFβ/Activin-Nodal signaling. In certain embodiments, the method further comprises contacting the cells with an effective amount of one or more agonist of an estrogen receptor (ER) (referred to as “ER agonist”).

In certain embodiments, the one or more dorsalizing agent comprises an activator of FGF signaling (referred to as “FGF activator”). Non-limiting examples of FGF activators include FGF1, FGF2, FGF3, FGF4, FGF7, FGF8, FGF10, FGF18, derivatives thereof, and mixtures thereof. In certain embodiments, the one or more FGF activator comprises FGF8.

In certain embodiments, the one or more ventralizing agent comprises an activator of BMP (referred to as “BMP activator”) or a BMP molecule, for example, BMP2. Non-limiting examples of BMP activators include BMP1, BMP2, BMP3, BMP4, BMP5, BMP6, BMP7, BMP8a, BMP8b, BMP10, BMP11, BMP15, derivatives thereof, and mixtures thereof. In certain embodiments, the one or more BMP activator comprises BMP2.

As used herein, the term “WNT” or “wingless” in reference to a ligand refers to a group of secreted proteins (i.e. Intl (integration 1) in humans) capable of interacting with a WNT receptor, such as a receptor in the Frizzled and LRPDerailed/RYK receptor family. As used herein, the term “WNT” or “wingless” in reference to a signaling pathway refers to a signal pathway composed of Wnt family ligands and Wnt family receptors, such as Frizzled and LRPDerailed/RYK receptors, mediated with or without β-catenin. In certain embodiments, a WNT signaling pathway includes mediation by β-catenin, e.g., WNT/-catenin.

In certain embodiments, the one or more Wnt activator lowers glycogen synthase kinase 3β (GSK3β) for activation of Wnt signaling. Thus, the Wnt activator can be a GSK3β inhibitor. A GSK3P inhibitor is capable of activating a WNT signaling pathway, see e.g., Cadigan, et al., J Cell Sci. 2006; 119:395-402; Kikuchi, et al., Cell Signaling. 2007; 19:659-671, which are incorporated by reference herein in their entireties. As used herein, the term “glycogen synthase kinase 3β inhibitor” refers to a compound that inhibits a glycogen synthase kinase 3β enzyme, for example, see, Doble, et al., J Cell Sci. 2003; 116:1175-1186, which is incorporated by reference herein in its entirety.

Non-limiting examples of Wnt activators or GSK3β inhibitors are disclosed in WO2011/149762, Chambers (2012), and Calder et al., J Neurosci. 2015 Aug. 19; 35 (33): 11462-81, which are incorporated by reference in their entireties. Non-limiting examples of Wnt activators include CHIR99021, WNT3A, Wnt-1, Wnt4, Wnt5a, derivatives thereof, and mixtures thereof. In certain embodiments, the one or more Wnt activator is a small molecule selected from the group consisting of CHIR99021, derivatives thereof, and mixtures thereof. In certain embodiments, the one or more Wnt activator comprises CHIR99021.

“CHIR99021” (also known as “aminopyrimidine” or “3-[3-(2-Carboxyethyl)-4-methylpyrrol-2-methylidenyl]-2-indolinone”) refers to IUPAC name 6-(2-(4-(2,4-dichlorophenyl)-5-(4-methyl-1H-imidazol-2-yl)pyrimidin-2-ylamino)ethylamino) nicotinonitrile with the following formula.

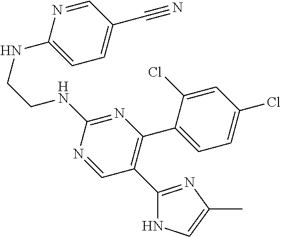 [SMILES: Cc1c[nH]c(-c2cnc(NCCNc3ccc(C#N)cn3)nc2-c2ccc(Cl)cc2Cl)n1]

CHIR99021 is highly selective, showing nearly thousand-fold selectivity against a panel of related and unrelated kinases, with an IC50=6.7 nM against human GSK3β and nanomolar IC50 values against rodent GSK3β homologs.

In certain embodiments, the one or more ER agonist comprises an agonist of ERβ. In certain embodiments, the one or more ER agonist comprises an agonist of ERα. Non-limiting examples of ERβ agonists include diarylpropionitrile (DPN), Estradiol (E2), propylpyrazole-triol (PPT), derivatives thereof, and mixtures thereof. In certain embodiments, the one or more ER agonist comprises DPN.

In certain embodiments, the cells (e.g., pituitary precursors, pituitary placode precursors) are initially contacted with the one or more dorsalizing agent and the one or more ventralizing agent at least about 5 days, at least about 6 days, at least about 7 days, at least about 8 days, at least about 9 days, at least about 10 days, at least about 11 days, at least about 12 days, at least about 13 days, at least about 14 days, at least about 15 days, or at least about 20 days from the initial contact of the stem cells with the one or more inhibitor of TGFβ/Activin-Nodal signaling. In certain embodiments, the cells (e.g., pituitary precursors, pituitary placode precursors) are initially contacted with the one or more dorsalizing agent and the one or more ventralizing agent at least about 5 days from the initial contact of the stem cells with the one or more inhibitor of TGFβ/Activin-Nodal signaling. In certain embodiments, the cells (e.g., pituitary precursors, pituitary placode precursors) are initially contacted with the one or more dorsalizing agent and the one or more ventralizing agent at least about 8 days from the initial contact of the stem cells with the one or more inhibitor of TGFβ/Activin-Nodal signaling. In certain embodiments, the cells (e.g., pituitary precursors, pituitary placode precursors) are initially contacted with the one or more dorsalizing agent and the one or more ventralizing agent about 8 days from the initial contact of the stem cells with the one or more inhibitor of TGFβ/Activin-Nodal signaling. In certain embodiments, the cells (e.g., pituitary precursors, pituitary placode precursors) are initially contacted with the one or more dorsalizing agent and the one or more ventralizing agent 9 days from the initial contact of the stem cells with the one or more inhibitor of TGFβ/Activin-Nodal signaling.

In certain embodiments, the cells (e.g., pituitary precursors, pituitary placode precursors) are contacted with the one or more dorsalizing agent for at least about 3 days, at least about 4 days, at least about 5 days, at least about 6 days, at least about 7 days, at least about 8 days, at least about 9 days, at least about 10 days, at least about 15 days, or at least about 20 days. In certain embodiments, the cells (e.g., pituitary precursors, pituitary placode precursors) are contacted with the one or more dorsalizing agent for up to about 4 days, up to about 5 days, up to about 6 days, up to about 7 days, up to about 8 days, up to about 9 days, or up to about 10 days. In certain embodiments, the cells (e.g., pituitary precursors, pituitary placode precursors) are contacted with the one or more dorsalizing agent for up to about 4 days or up to about 5 days. In certain embodiments, the cells (e.g., pituitary precursors, pituitary placode precursors) are contacted with the one or more dorsalizing agent for up to about 10 days. In certain embodiments, the cells (e.g., pituitary precursors, pituitary placode precursors) are contacted with the one or more dorsalizing agent for at least about 3 days. In certain embodiments, the cells (e.g., pituitary precursors, pituitary placode precursors) are contacted with the one or more dorsalizing agent for at least about 3 days and/or up to about 10 days. In certain embodiments, the cells (e.g., pituitary precursors, pituitary placode precursors) are contacted with the one or more dorsalizing agent for about 4 days. In certain embodiments, the cells (e.g., pituitary precursors, pituitary placode precursors) are contacted with the one or more dorsalizing agent for 5 days. In certain embodiments, the cells (e.g., pituitary precursors, pituitary placode precursors) are contacted with the one or more dorsalizing agent for at least about 5 days. In certain embodiments, the cells (e.g., pituitary precursors, pituitary placode precursors) are contacted with the one or more dorsalizing agent for about 7 days. In certain embodiments, the cells (e.g., pituitary precursors, pituitary placode precursors) are contacted with the one or more dorsalizing agent for 8 days.

In certain embodiments, the cells are contacted with the one or more ventralizing agent for at least about 5 days, at least about 6 days, at least about 7 days, at least about 8 days, at least about 9 days, at least about 10 days, at least about 15 days, or at least about 20 days. In certain embodiments, the cells are contacted with the one or more ventralizing agent for up to about 7 days, up to about 8 days, up to about 9 days, up to about 10 days, up to about 11 days, up to about 12 days, up to about 13 days, up to about 14 days, up to about 15 days, up to about 16 days, up to about 17 days, up to about 18 days, up to about 19 days, or up to about 20 days. In certain embodiments, the cells are contacted with the one or more ventralizing agent for up to about 8 days. In certain embodiments, the cells are contacted with the one or more ventralizing agent for up to about 15 days. In certain embodiments, the cells are contacted with the one or more ventralizing agent for up to about 12 days. In certain embodiments, the cells are contacted with the one or more ventralizing agent for at least about 5 days. In certain embodiments, the cells are contacted with the one or more ventralizing agent for about 7 days. In certain embodiments, the cells are contacted with the one or more ventralizing agent for 8 days. In certain embodiments, the cells are contacted with the one or more ventralizing agent for at least about 10 days. In certain embodiments, the cells are contacted with the one or more ventralizing agent for about 11 days. In certain embodiments, the cells (e.g., pituitary precursors, pituitary placode precursors) are contacted with the one or more ventralizing agent for 12 days.

In certain embodiments, the cells (e.g., pituitary precursors, pituitary placode precursors) are further contacted with or exposed to one or more inhibitor of TGFβ/Activin-Nodal signaling. In certain embodiments, the cells (e.g., pituitary precursors, pituitary placode precursors) are contacted with or exposed to the one or more dorsalizing agent and the one or more inhibitor of TGFβ/Activin-Nodal signaling concurrently. In certain embodiments, the cells (e.g., pituitary precursors, pituitary placode precursors) are contacted with the one or more inhibitor of TGFβ/Activin-Nodal signaling for at least about 3 days, at least about 4 days, at least about 5 days, at least about 6 days, at least about 7 days, at least about 8 days, at least about 9 days, at least about 10 days, at least about 15 days, or at least about 20 days. In certain embodiments, the cells (e.g., pituitary precursors, pituitary placode precursors) are contacted with the one or more inhibitor of TGFβ/Activin-Nodal signaling for up to about 4 days, up to about 5 days, up to about 6 days, up to about 7 days, up to about 8 days, up to about 9 days, or up to about 10 days. In certain embodiments, the cells (e.g., pituitary precursors, pituitary placode precursors) are contacted with the one or more inhibitor of TGFβ/Activin-Nodal signaling for up to about 4 days or up to about 5 days. In certain embodiments, the cells (e.g., pituitary precursors, pituitary placode precursors) are contacted with the one or more inhibitor of TGFβ/Activin-Nodal signaling for up to about 10 days. In certain embodiments, the cells (e.g., pituitary precursors, pituitary placode precursors) are contacted with the one or more inhibitor of TGFβ/Activin-Nodal signaling for at least about 3 days and/or up to about 10 days. In certain embodiments, the cells (e.g., pituitary precursors, pituitary placode precursors) are contacted with the one or more inhibitor of TGFβ/Activin-Nodal signaling for about 4 days. In certain embodiments, the cells (e.g., pituitary precursors, pituitary placode precursors) are contacted with the one or more inhibitor of TGFβ/Activin-Nodal signaling for at least about 5 days. In certain embodiments, the cells (e.g., pituitary precursors, pituitary placode precursors) are contacted with the one or more inhibitor of TGFβ/Activin-Nodal signaling for about 7 days. In certain embodiments, the cells (e.g., pituitary precursors, pituitary placode precursors) are contacted with the one or more inhibitor of TGFβ/Activin-Nodal signaling for 8 days.

In certain embodiments, the initial contact of the cells with the one or more Wnt activator (and optionally the one or more ER agonist) occurs on the same day as the initial contact of the cells (e.g., pituitary precursors, pituitary placode precursors) with the one or more dorsalizing agent. In certain embodiments, the cells are initially contacted with the one or more Wnt activator (and optionally the one or more ER agonist) at least about 1 day, at least about 2 days, at least about 3 days, at least about 4 days, at least about 5 days, at least about 6 days, at least about 7 days, at least about 8 days, at least about 9 days, or at least about 10 days, and/or no later than about 3 days, no later than about 4 days, no later than about 5 days, no later than about 6 days, no later than about 7 days, no later than about 8 days, no later than about 9 days, or no later than about 10 days from the initial contact of the cells (e.g., pituitary precursors, pituitary placode precursors) with the one or more dorsalizing agent. In certain embodiments, the cells are initially contacted with the one or more Wnt activator (and optionally the one or more ER agonist) at least about 2 days from the initial contact of the cells (e.g., pituitary precursors, pituitary placode precursors) with the one or more dorsalizing agent. In certain embodiments, the cells are initially contacted with the one or more Wnt activator (and optionally the one or more ER agonist) no later than about 5 days from the initial contact of the cells (e.g., pituitary precursors, pituitary placode precursors) with the one or more dorsalizing agent. In certain embodiments, the cells are initially contacted with the one or more Wnt activator (and optionally the one or more ER agonist) at least about 2 days and no later than about 5 days from the initial contact of the cells (e.g., pituitary precursors, pituitary placode precursors) with the one or more dorsalizing agent. In certain embodiments, the cells are initially contacted with the one or more Wnt activator (and optionally the one or more ER agonist) about 4 days from the initial contact of the cells (e.g., pituitary precursors, pituitary placode precursors) with the one or more dorsalizing agent. In certain embodiments, the cells are initially contacted with the one or more Wnt activator (and optionally the one or more ER agonist) 5 days from the initial contact of the cells (e.g., pituitary precursors, pituitary placode precursors) with the one or more dorsalizing agent.

In certain embodiments, the cells are contacted with the one or more Wnt activator (and optionally the one or more ER agonist) for at least about 5 days, at least about 6 days, at least about 7 days, at least about 8 days, at least about 9 days, at least about 10 days, at least about 15 days, or at least about 20 days. In certain embodiments, the cells are contacted with the one or more Wnt activator (and optionally the one or more ER agonist) for up to about 7 days, up to about 8 days, up to about 9 days, up to about 10 days, up to about 11 days, up to about 12 days, up to about 13 days, up to about 14 days, up to about 15 days, up to about 16 days, up to about 17 days, up to about 18 days, up to about 19 days, or up to about 20 days. In certain embodiments, the cells are contacted with the one or more Wnt activator (and optionally the one or more ER agonist) for up to about 10 days. In certain embodiments, the cells are contacted with the one or more Wnt activator (and optionally the one or more ER agonist) for up to about 8 days. In certain embodiments, the cells are contacted with the one or more Wnt activator (and optionally the one or more ER agonist) for at least about 5 days. In certain embodiments, the cells are contacted with the one or more Wnt activator (and optionally the one or more ER agonist) for at least about 5 days and up to about 10 days. In certain embodiments, the cells are contacted with the one or more Wnt activator (and optionally the one or more ER agonist) for at least about 7 days. In certain embodiments, the cells are contacted with the one or more Wnt activator (and optionally the one or more ER agonist) for about 7 days. In certain embodiments, the cells are contacted with the one or more Wnt activator (and optionally the one or more ER agonist) for 8 days.

In certain embodiments, the cells are contacted with (exposed to) the one or more dorsalizing agent, the one or more ventralizing agent, and the one or more Wnt activator, and optionally the one or more ER agonist) concurrently.

In certain embodiments, the cells (e.g., pituitary precursors, pituitary placode precursors) are contacted with the one or more dorsalizing agent at a concentration of between about 10 ng/mL and 200 ng/mL, between about 20 ng/mL and 150 ng/mL, between about 50 ng/ml and 150 ng/mL, between about 150 ng/mL and 200 ng/mL, between about 30 ng/ml and 100 ng/ml, between about 50 ng/mL and 100 ng/mL, between about 40 ng/mL and 75 ng/ml or between about 50 ng/ml and 75 ng/mL. In certain embodiments, the cells (e.g., pituitary precursors, pituitary placode precursors) are contacted with the one or more dorsalizing agent at a concentration of about 50 ng/mL, or about 100 ng/mL. In certain embodiments, the cells (e.g., pituitary precursors, pituitary placode precursors) are contacted with the one or more dorsalizing agent at a concentration of about 50 ng/mL. In certain embodiments, the cells (e.g., pituitary precursors, pituitary placode precursors) are contacted with FGF8 at a concentration of about 50 ng/mL. In certain embodiments, the cells (e.g., pituitary precursors, pituitary placode precursors) are contacted with the one or more dorsalizing agent at a concentration of about 100 ng/ml. In certain embodiments, the cells (e.g., pituitary precursors, pituitary placode precursors) are contacted with FGF8 at a concentration of about 100 ng/mL.

In certain embodiments, the cells (e.g., pituitary precursors, pituitary placode precursors) are contacted with the one or more ventralizing agent at a concentration of between about 1 ng/mL and 30 ng/mL, between about 5 ng/mL and 25 ng/mL, or between about 10 ng/mL and 20 ng/mL. In certain embodiments, the cells (e.g., pituitary precursors, pituitary placode precursors) are contacted with the one or more ventralizing agent at a concentration of about 10 ng/ml, or about 20 ng/mL. In certain embodiments, the cells (e.g., pituitary precursors, pituitary placode precursors) are contacted with the one or more ventralizing agent at a concentration of about 20 ng/mL. In certain embodiments, the cells (e.g., pituitary precursors, pituitary placode precursors) are contacted with BMP2 at a concentration of about 20 ng/mL.

In certain embodiments, the cells (e.g., pituitary precursors, pituitary placode precursors) are contacted with the inhibitor of TGFβ/Activin-Nodal signaling at a concentration of between about 1 μM and 20 μM, between about 2 μM and 18 μM, between about 4 μM and 16 μM, between about 6 μM and 14 μM, or between about 8 UM and 12 μM. In certain embodiments, the cells (e.g., pituitary precursors, pituitary placode precursors) are contacted with the inhibitor of TGFβ/Activin-Nodal signaling at a concentration of about 10 μM. In certain embodiments, the cells are contacted with SB43152 at a concentration of about 10 μM.

In certain embodiments, the cells are contacted with the one or more Wnt activator agent at a concentration of from about 1 μM to 100 μM, from about 1 μM to 20 μM, from about 1 μM to 15 μM, from about 1 μM to 10 μM, from about 1 μM to 5 μM, from about 5 μM to 10 μM, from about 5 μM to 15 μM, from about 15 μM to 20 μM, from about 20 μM to 30 μM, from about 30 μM to 40 μM, from about 40 μM to 50 μM, from about 50 μM to 60 μM, from about 60 μM to 70 μM, from about 70 μM to 80 μM, from about 80 μM to 90 μM, or from about 90 μM to 100 μM. In certain embodiments, the cells are contacted with the one or more Wnt activator at a concentration of from about 1 μM to 10 μM. In certain embodiments, the cells are contacted with the one or more Wnt activator at a concentration of from about 1 μM to 5 μM. In certain embodiments, the cells are contacted with the one or more Wnt activator at a concentration of about 3 μM. In certain embodiments, the cells are contacted with CHIR99021 at a concentration of about 3 μM.

In certain embodiments, the cells are contacted with the one or more ER agonist at a concentration of between about 0.1 nM and 20 nM, between about 0.1 nM and 10 nM, between about 0.1 nM and 1 nM, or between about 0.1 nM and 0.5 nM. In certain embodiments, the cells are contacted with the one or more ER agonist at a concentration of between about 0.1 nM and 0.5 nM. In certain embodiments, the cells are contacted with the one or more ER agonist at a concentration of about 0.1 nM. In certain embodiments, the cells are contacted with DPN at a concentration of about 0.1 nM.

In certain embodiments, the cells are contacted with (or exposed to) each of the one or more dorsalizing agent, the one or more ventralizing agent, the one or more Wnt activator, the one or more ER agonist, and the one or more inhibitor of TGFβ/Activin-Nodal signaling daily. In certain embodiments, the cells are contacted with (or exposed to) each of the one or more dorsalizing agent, the one or more ventralizing agent, the one or more Wnt activator, the one or more ER agonist, and the one or more inhibitor of TGFβ/Activin-Nodal signaling every other day. In certain embodiments, the method comprises contacting the cells with the one or more dorsalizing agent, the one or more ventralizing agent, and the one or more Wnt activator, and optionally the one or more ER agonist and one or more inhibitor of TGFβ/Activin-Nodal signaling to obtain a first cell population of differentiated cells, wherein greater than about 50%, greater than about 60%, greater than about 70%, greater than about 80%, or greater than about 90% of the differentiated cells express a detectable level of Pit1 at least about 5 days, at least about 6 days, at least about 7 days, at least about 8 days, at least about 9 days, at least about 10 days, at least about 11 days, at least about 12 days, at least about 13 days, at least about 14 days, or about 15 days from the initial contact of the cells (e.g., pituitary precursors, pituitary placode precursors) with the one or more dorsalizing agent. In certain embodiments, greater than about 70% of the differentiated cells express a detectable level of Pit1. In certain embodiments, the method comprises obtaining the first cell population at least about 5 days from the initial contact of the cells (e.g., pituitary precursors, pituitary placode precursors) with the one or more dorsalizing agent. In certain embodiments, the method comprises obtaining the first cell population about 7 days (e.g., 7 days or 8 days), about 10 days (e.g., 11 days or 12 days) from the initial contact of the cells (e.g., pituitary precursors, pituitary placode precursors) with the one or more dorsalizing agent.

In certain embodiments, the method comprises obtaining the first cell population at least about 10 days, at least about 11 days, at least about 12 days, at least about 13 days, at least about 14 days, about 15 days, at least about 16 days, at least about 17 days, at least about 18 days, at least about 19 days, or at least about 20 days from the initial contact of the stem cells with the one or more inhibitor of TGFβ/Activin-Nodal signaling. In certain embodiments, the method comprises obtaining the first cell population at least about 10 days from the initial contact of the cells with the one or more inhibitor of TGFβ/Activin-Nodal signaling. In certain embodiments, the method comprises obtaining the first cell population about 15 days, about 16 days, about 17 days, about 18 days, about 19 days or about 20 days from the initial contact of the cells with the one or more inhibitor of TGFβ/Activin-Nodal signaling.

In certain embodiments, the method comprises contacting cells (e.g., cells expressing one or more pituitary precursor marker disclosed herein) with the one or more dorsalizing agent and the one or more ventralizing agent, and optionally the one or more inhibitor of TGFβ/Activin-Nodal signaling to obtain a second cell population of differentiated cells, wherein greater than about 50%, greater than about 60%, greater than about 70%, greater than about 80%, or greater than about 90% of the differentiated cells express a detectable level of Prop1 at least about 2 days, about 3 days, about 4 days, or about 5 days from the initial contact of the cells with the one or more dorsalizing agent. In certain embodiments, the method comprises obtaining the second cell population at least about 2 days (e.g., about 4 days or about 5 days) from the initial contact of the cells (e.g., pituitary precursors, pituitary placode precursors) with the one or more dorsalizing agent.

In certain embodiments, the method comprises obtaining the second cell population at least about 10 days, at least about 11 days, at least about 12 days, at least about 13 days, at least about 14 days, or at least about 15 days from the initial contact of the stem cells with the one or more inhibitor of TGFβ/Activin-Nodal signaling. In certain embodiments, the method comprises obtaining the second cell population at least about 8 days from the initial contact of the stem cells with the one or more inhibitor of TGFβ/Activin-Nodal signaling. In certain embodiments, the method comprises obtaining the second cell population about 12 days from the initial contact of the stem cells with the one or more inhibitor of TGFβ/Activin-Nodal signaling.

In certain embodiments, the method comprises contacting the second cell population with the one or more ventralizing agent and the one or more Wnt activator, and optionally the one or more ER agonist to obtain the first cell population. In certain embodiments, the first cell population is obtained at least about 5 days, at least about 6 days, at least about 7 days, at least about 8 days, at least about 9 days, at least about 10 days, at least about 11 days, at least about 12 days, at least about 13 days, at least about 14 days, or at least about 15 days from the presence of the second cell population. In certain embodiments, the first cell population is obtained at least about 5 days from the presence of the second cell population. In certain embodiments, the first cell population is obtained about 7 days from the presence of the second cell population. In certain embodiments, the first cell population is obtained about 8 days from the presence of the second cell population. In certain embodiments, the method comprises contacting the cells (e.g., cells expressing one or more pituitary precursor marker disclosed herein) with the one or more dorsalizing agent (e.g., 50 ng/mL FGF8), the one or more ventralizing agent (e.g., 20 ng/mL BMP2), the one or more Wnt activator (e.g., 3 μM CHIR99021), and the one or more ER agonist (e.g., 0.1 nM DPN) to obtain the first cell population, e.g., at about 7 days or about 8 days from the initial contact of the cells (e.g., cells expressing one or more pituitary precursor marker disclosed herein) with the one or more dorsalizing agent, and/or about 15 days or about 16 days from the initial contact of the stem cells with the one or more inhibitor of TGFβ/Activin-Nodal signaling. In certain embodiments, the initial contact of the cells (e.g., cells expressing one or more pituitary precursor marker disclosed herein) with the dorsalizing agent(s), ventralizing agent(s), Wnt activator(s) and ER agonist(s) is about 8 days or about 9 days) from the initial contact of the stem cells with the one or more inhibitor of TGFβ/Activin-Nodal signaling.

In certain embodiments, the method comprises contacting the cells expressing one or more pituitary precursor marker (e.g., those disclosed herein) with the one or more dorsalizing agent (e.g., 50 ng/mL FGF8 or 100 ng/mL), the one or more ventralizing agent (e.g., 20 ng/ml BMP2), and the one of more inhibitor of TGFβ/Activin-Nodal signaling (e.g., 10 μM SB43152); and contacting the cells with the one or more Wnt activator (e.g., 3 μM CHIR99021) and the one or more ER agonist (e.g., 0.1 nM DPN) to obtain the first cell population, for example, at least about 10 days (e.g., about 10 days, about 11 days, or about 12 days) from the initial contact of the cells expressing one or more pituitary precursor marker with the one or more dorsalizing agent, and/or at least about 15 days (e.g., about 19 days or about 20 days) from the initial contact of the stem cells with the one of more inhibitor of TGFβ/Activin-Nodal signaling.

In certain embodiments, the method comprises contacting the cells expressing one or more pituitary precursor marker (e.g., those disclosed herein) with the one or more dorsalizing agent (e.g., 50 ng/mL FGF8 or 100 ng/mL FGF8), the one or more ventralizing agent (e.g., 20 ng/mL BMP2), and the one of more inhibitor of TGFβ/Activin-Nodal signaling (e.g., 10 μM SB43152) to obtain the second cell population, for example, at least about 4 days (e.g., about 4 days or about 5 days) from the initial contact of the cells expressing one or more pituitary precursor marker with the one or more dorsalizing agent, and/or at least about 10 days (e.g., about 10 days, about 11 days, or about 12 days) from the initial contact of the stem cells with the one of more inhibitor of TGFβ/Activin-Nodal signaling.

In certain embodiments, the method comprises contacting the second cell population with the one or more ventralizing agent (e.g., 20 ng/mL BMP2), and the one or more Wnt activator (e.g., 3 μM CHIR99021), and the one or more ER agonist (e.g., 0.1 nM DPN) to obtain the first cell population, for example, at least about 7 days (e.g., about 7 days or about 8 days) from the presence of the second cell population.

### 5.2.3. Method of Differentiating Pit1⁺ Cells to GH-Producing Somatotrophs

In certain embodiments, the Pit1⁺ cells (e.g., the differentiated cells obtained from the method described in Section 5.2.2) or the first cell population disclosed in section 5.2.2 are differentiated into GH-producing somatotrophs. In certain embodiments, the Pit1⁺ cells are or the first cell population is differentiated into a cell population comprising at least about 50%, at least about 60%, at least about 70%, at least about 80%, or at least about 90%, and up to about 100% GH-producing somatotrophs, wherein the Pit1⁺ cells are or the first cell population is contacted with (or exposed to) an effective amount of one or more molecule that is capable of inducing growth hormone (GH) expression (GH inducer). The GH inducers can suppress non-GH lineage cells, e.g., thyrotrophs, and lactotroph.

Non-limiting examples of GH inducers include retinoic acid (RA), corticosteroids, thyroid hormones, ER agonists, GHRH signaling pathway agonists, Ghrelin signaling pathway agonist, and interleukins, and derivatives thereof, and mixtures thereof.

Non-limiting examples of corticosteroids include dexamethasone, cortisone, hydrocortisone, derivatives thereof, and mixtures thereof. In certain embodiments, the one or more corticosteroid includes dexamethasone.

Non-limiting examples of thyroid hormones include T3, T4, derivatives thereof, and mixtures thereof. In certain embodiments, the one or more thyroid hormone includes T3.

Non-limiting examples of GHRH signaling pathway agonists include GHRH, c-AMP (e.g., Dibutyryl-cAMP), PKA, CREB, MAPK activator, derivatives thereof, and mixtures thereof. In certain embodiments, the one or more GHRH signaling pathway agonist includes GHRH, c-AMP, or a combination thereof.

Non-limiting examples of Ghrelin signaling pathway agonists include Ghrelin, GHSR agonists, derivatives thereof and mixtures thereof. In certain embodiments, the one or more Ghrelin signaling pathway agonist includes Ghrelin.

Non-limiting examples of interleukins include IL-1, IL-2, IL-3, IL-4, IL-5, IL-6, IL-7, IL-8, IL-9, IL-10, IL-11, IL-12, IL-13, IL-15, and combinations thereof. In certain embodiments, the one or more interleukin is selected from the group consisting of IL-1, IL6, IL-10, and combinations thereof. In certain embodiments, the one or more interleukin includes IL-6.

RA can suppress POMC expressed by corticotrophs and promote the expression of GH1, which is expressed by somatotrophs. Corticosteroid (e.g., dexamethasone) and thyroid hormone (e.g., T3) can inhibit PRL (expressed by lactotroph) and/or TSH (expressed by thyrotroph) while promoting GH1 expression.

In certain embodiments, the Pit1⁺ cells are or the first cell population is contacted with one, two, three, four, five, six, or seven GH inducers. In certain embodiments, the Pit1⁺ cells are or the first cell population is contacted with four GH inducers, e.g., RA, corticosteroid, thyroid hormone, and one GHRH signaling pathway agonist (e.g., GHRH). In certain embodiments, the Pit1⁺ cells are or the first cell population is contacted with five GH inducers, e.g., RA, corticosteroid, thyroid hormone, and two GHRH signaling pathway agonists (e.g., GHRH and CAMP). In certain embodiments, the Pit1⁺ cells are or the first cell population is contacted with five GH inducers, e.g., RA, corticosteroid, thyroid hormone, one GHRH signaling pathway agonist (e.g., GHRH), and one Ghrelin signaling pathway agonist (e.g., Ghrelin). In certain embodiments, the Pit1⁺ cells are or the first cell population is contacted with six GH inducers, e.g., RA, corticosteroid, thyroid hormone, and two GHRH signaling pathway agonists (e.g., GHRH and cAMP), and one ER agonist (e.g., DPN). In certain embodiments, the Pit1⁺ cells are or the first cell population is contacted with six GH inducers, e.g., RA, corticosteroid, thyroid hormone, one GHRH signaling pathway agonist (e.g., GHRH), one Ghrelin signaling pathway agonist (e.g., Ghrelin), and one ER agonist (e.g., DPN).

In certain embodiments, the cells are contacted with RA at a concentration of from about 0.05 μM to 10 μM, from about 0.05 μM to 0.1 μM, from about 0.1 μM to 10 μM, from about 0.1 μM to 5 μM, from about 0.1 μM to 3 μM, from about 0.1 μM to 2 μM, from about 0.1 μM to 1 μM, or from about 5 μM to 10 μM. In certain embodiments, the cells are contacted with RA at a concentration of from about 0.1 μM to 1 μM. In certain embodiments, the cells are contacted with RA at a concentration of from about 0.05 μM to 0.1 μM. In certain embodiments, the cells are contacted with RA at a concentration of about 1 μM. In certain embodiments, the cells are contacted with RA at a concentration of about 0.1 μM.

In certain embodiments, the cells are contacted with one or more thyroid hormone at a concentration of between about 1 nM and 20 nM, between about 2 nM and 18 nM, between about 4 nM and 16 nM, between about 6 nM and 14 nM, or between about 8 nM and 12 nM. In certain embodiments, the cells are contacted with one or more thyroid hormone at a concentration of about 10 nM. In certain embodiments, the cells are contacted with T3 at a concentration of about 10 nM.

In certain embodiments, the cells are contacted with one or more corticosteroid at a concentration of from about 0.1 μM to 10 UM, from about 0.1 μM to 5 μM, from about 0.1 μM to 3 μM, from about 0.1 μM to 2 μM, from about 0.1 μM to 1 μM, or from about 5 μM to 10 μM. In certain embodiments, the cells are contacted with the one or more corticosteroid at a concentration of from about 0.1 μM to 10 μM. In certain embodiments, the cells are contacted with the one or more corticosteroid at a concentration of from about 0.1 μM to 1 μM. In certain embodiments, the cells are contacted with the corticosteroid at a concentration of about 1 μM. In certain embodiments, the cells are contacted with dexamethasone at a concentration of about 1 μM.

In certain embodiments, the cells are contacted with one or more GHRH signaling pathway agonist at a concentration of from about 0.01 μM to 10 UM, from about 0.01 μM to 0.05 μM, from about 0.01 μM to 0.1 μM, from about 0.1 μM to 10 μM, from about 0.1 μM to 5 μM, from about 0.1 μM to 3 μM, from about 0.1 μM to 2 μM, from about 0.1 μM to 1 μM, from about 5 μM to 10 μM, between about 10 μg/mL and 200 μg/mL, between about 20 μg/mL and 150 μg/mL, between about 50 μg/mL and 150 μg/mL, between about 50 μg/mL and 100 μg/mL, or between about 100 μg/mL and 200 μg/mL. In certain embodiments, the cells are contacted with the one or more GHRH signaling pathway agonist at a concentration of about 1 μM. In certain embodiments, the cells are contacted with GHRH at a concertation of about 1 μM. In certain embodiments, the cells are contacted with the one or more GHRH signaling pathway agonist at a concentration of about 0.01 μM. In certain embodiments, the cells are contacted with GHRH at a concertation of about 0.01 μM. In certain embodiments, the cells are contacted with the one or more GHRH signaling pathway agonist at a concentration of about 100 μg/mL. In certain embodiments, the cells are contacted with cAMP at a concertation of about 100 μg/mL.

In certain embodiments, the cells are contacted with one or more ER agonist at a concentration of between about 0.1 nM and 20 nM, between about 0.1 nM and 10 nM, between about 0.1 nM and 1 nM, or between about 0.1 nM and 0.5 nM. In certain embodiments, the cells are contacted with the one or more ER agonist at a concentration of between about 0.1 nM and about 0.5 nM. In certain embodiments, the cells are contacted with the one or more ER agonist at a concentration of about 0.1 nM. In certain embodiments, the cells are contacted with DPN at a concentration of about 0.1 nM.

In certain embodiments, the cells are contacted with one or more Ghrelin signaling pathway agonist at a concentration of between about 1 nM and 100 nM, between about 1 nM and 50 nM, between about 1 nM and 30 nM, between about 1 nM and 20 nM, between about 1 nM and 15 nM, between about 5 nM and 20 nM, between about 5 nM and 15 nM, between about 20 nM and 30 nM, between about 30 nM and 40 nM, between about 40 nM and 50 nM, or between about 50 nM and 100 nM. In certain embodiments, the cells contacted with the one or more Ghrelin signaling pathway agonist at a concentration of between about 5 nM and 15 nM. In certain embodiments, the cells are contacted with the one or more Ghrelin signaling pathway agonist at a concentration of about 10 nM. In certain embodiments, the cells are contacted with Ghrelin at a concentration of about 10 nM.

In certain embodiments, the cells are contacted with one or more interleukins at a concentration of between about 1 ng/ml and 100 ng/ml, between about 1 ng/ml and 50 ng/ml, between about 1 ng/ml and 30 ng/ml, between about 1 ng/ml and 25 ng/ml, between about 5 ng/ml and 30 ng/ml, between about 10 ng/ml and 30 ng/ml, between about 10 ng/ml and 25 ng/ml, between about 20 ng/ml and 25 ng/ml, between about 30 ng/ml and 40 ng/ml, between about 40 ng/ml and 50 ng/ml, or between about 50 ng/ml and 100 ng/ml. In certain embodiments, the cells are contacted with the one or more interleukins at a concentration of between about 10 ng/ml and 30 ng/ml. In certain embodiments, the cells are contacted with the one or more interleukins at a concentration of about 25 ng/ml. In certain embodiments, cells are contacted with IL-6 at a concentration of about 25 ng/ml.

In certain embodiments, the cells are contacted with (or exposed to) the one or more GH inducer daily. In certain embodiments, the cells are contacted with (or exposed to) the one or more GH inducer every other day.

In certain embodiments, the cells (e.g., Pit1⁺ cells) are contacted with the one or more GH inducer for at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 1 week, at least about 2 weeks, at least about 3 weeks, at least about 4 weeks, at least about 5 weeks, at least about 6 weeks, or at least about 7 weeks. In certain embodiments, the cells (e.g., Pit1⁺ cells) are contacted with the one or more GH inducer for at least about 5 days. In certain embodiments, the Pit1⁺ cells are contacted with the GH inducer for at least about 6 days. In certain embodiments, the cells (e.g., Pit1⁺ cells) are contacted with the one or more GH inducer for at least about 14 days. In certain embodiments, the cells (e.g., Pit1⁺ cells) are contacted with the one or more GH inducer for at least about 15 days. In certain embodiments, the cells (e.g., Pit1⁺ cells) are contacted with the one or more GH inducer for at least about 19 days. In certain embodiments, the Pit1⁺ cells are contacted with the one or more GH inducer for at least about 20 days. In certain embodiments, the cells (e.g., Pit1⁺ cells) are contacted with the one or more GH inducer for at least about 2 weeks. In certain embodiments, the cells (e.g., Pit1⁺ cells) are contacted with the one or more GH inducer for at least about 4 weeks. In certain embodiments, the cells (e.g., Pit1⁺ cells) are contacted with the one or more GH inducer for at least about 6 weeks. In certain embodiments, the cells (e.g., Pit1⁺ cells) are contacted with the one or more GH inducer for up to about 5 days, up to about 6 days, up to about 7 days, up to about 8 days, up to about 9 days, up to about 10 days, up to about 11 days, up to about 12 days, up to about 13 days, up to about 14 days, up to about 15 days, up to about 16 days, up to about 17 days, up to about 18 days, up to about 19 days, up to about 20 days, up to about 1 week, up to about 2 weeks, up to about 3 weeks, up to about 4 weeks, up to about 5 weeks, up to about 6 weeks, up to about 7 weeks, up to about 8 weeks, up to about 9 weeks, or up to about 10 weeks. In certain embodiments, the cells (e.g., Pit1⁺ cells) are contacted with the one or more GH inducer for up to about 5 days. In certain embodiments, the cells (e.g., Pit1⁺ cells) are contacted with the one or more GH inducer for up to about 6 days. In certain embodiments, the cells (e.g., Pit1⁺ cells) are contacted with the one or more GH inducer for up to about 14 days. In certain embodiments, the cells (e.g., Pit1⁺ cells) are contacted with the one or more GH inducer for up to about 15 days. In certain embodiments, the cells (e.g., Pit1⁺ cells) are contacted with the one or more GH inducer for up to about 19 days. In certain embodiments, the cells (e.g., Pit1⁺ cells) are contacted with the one or more GH inducer for up to about 20 days. In certain embodiments, the cells (e.g., Pit1⁺ cells) are contacted with the one or more GH inducer for up to about 4 weeks. In certain embodiments, the cells (e.g., Pit1⁺ cells) are contacted with the one or more GH inducer for up to about 6 weeks. In certain embodiments, the cells (e.g., Pit1⁺ cells) are contacted with the one or more GH inducer for about 5 days. In certain embodiments, the cells (e.g., Pit1⁺ cells) are contacted with the one or more GH inducer for about 6 days. In certain embodiments, the cells (e.g., Pit1⁺ cells) are contacted with the one or more GH inducer for about 14 days. In certain embodiments, the cells (e.g., Pit1⁺ cells) are contacted with the one or more GH inducer for about 15 days. In certain embodiments, the cells (e.g., Pit1⁺ cells) are contacted with the one or more GH inducer for about 19 days. In certain embodiments, the cells (e.g., Pit1⁺ cells) are contacted with the one or more GH inducer for about 20 days. In certain embodiments, the cells (e.g., Pit1⁺ cells) are contacted with the one or more GH inducer for about 2 weeks. In certain embodiments, the cells (e.g., Pit1⁺ cells) are contacted with the one or more GH inducer for about 4 weeks. In certain embodiments, the cells (e.g., Pit1⁺ cells) are contacted with the one or more GH inducer for about 6 weeks.

In certain embodiments, the cells (e.g., Pit1⁺ cells) are or the first cell population is initially contacted with the one or more GH inducer at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 2 weeks, or at least about 3 weeks, and/or up to about 15 days, up to about 16 days, up to about 17 days, up to about 18 days, up to about 19 days, up to about 20 days, up to about 21 days, up to about 22 days, up to about 23 days, up to about 24 days, or up to about 25 days from the initial contact of the stem cells with the one or more inhibitor of TGFβ/Activin-Nodal signaling. In certain embodiments, the Pit1⁺ cells are or the first cell population is initially contacted with the one or more GH inducer at least about 10 days from the initial contact of the stem cells with the one or more inhibitor of TGFβ/Activin-Nodal signaling. In certain embodiments, the Pit1⁺ cells are or the first cell population is initially contacted with the one or more GH inducer at least about 15 days from the initial contact of the stem cells with the one or more inhibitor of TGFβ/Activin-Nodal signaling. In certain embodiments, the Pit1⁺ cells are or the first cell population is initially contacted with the one or more GH inducer no later than 25 days from the initial contact of the stem cells with the one or more inhibitor of TGFβ/Activin-Nodal signaling. In certain embodiments, the Pit1⁺ cells are or the first cell population is initially contacted with the one or more GH inducer no later than 20 days from the initial contact of the stem cells with the one or more inhibitor of TGFβ/Activin-Nodal signaling. In certain embodiments, the Pit1⁺ cells are or the first cell population is initially contacted with the one or more GH inducer at least about 15 days (e.g., about 15 days, about 16 days, about 17 days, about 18 days, about 19 days, or about 20 days) from the initial contact of the stem cells with the one or more inhibitor of TGFβ/Activin-Nodal signaling. In certain embodiments, the Pit1⁺ cells are or the first cell population is initially contacted with the one or more GH inducer at least about 2 weeks (e.g., about 2 weeks, about 15 days, about 16 days, about 17 days, about 18 days, about 19 days, or about 20 days) from the initial contact of the stem cells with the one or more inhibitor of TGFβ/Activin-Nodal signaling.

In certain embodiments, the GH-producing somatotrophs express a detectable level of one or more somatotroph marker. Non-limiting examples of somatotroph markers include GH1, GHRH receptor (GHRHR), POU1F1, NeuroD4, and GHSR. The cells expressing one or more somatotroph marker can secret GH.

In certain embodiments, the GH-producing somatotrophs express a detectable level of one or more somatotroph marker at least about 3 days, at least about 4 days, at least about 5 days, at least about 6 days, at least about 7 days, at least about 8 days, at least about 9 days, at least about 10 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 1 week, at least about 2 weeks, at least about 3 weeks, or at least about 4 weeks from the initial contact of the cells (e.g., Pit1⁺ cells) or the first cell population with the one or more GH inducer. In certain embodiments, the GH-producing somatotrophs express a detectable level of one or more somatotroph marker at least about 5 days (e.g., about 5 days or 6 days) from the initial contact of the cells (e.g., Pit1⁺ cells) or the first cell population with the one or more GH inducer. In certain embodiments, the GH-producing somatotrophs express a detectable level of one or more somatotroph marker at least about 15 days (e.g., about 14 days or 15 days) from the initial contact of the cells or the first cell population with the one or more GH inducer. In certain embodiments, the GH-producing somatotrophs express a detectable level of one or more somatotroph marker at least about 2 weeks from the initial contact of the cells (e.g., Pit1⁺ cells) or the first cell population with the one or more GH inducer.

In certain embodiments, the method comprises contacting the Pit1⁺ cells or the first cell population with one or more GH inducer to obtain a third cell population of differentiated cells, wherein at least about 50% (e.g., at least about 60%, at least about 70%, at least about 80%, at least about 90%, at least about 95%, or at least about 99%) of the differentiated cells express a low level of GHRHR immunoreactivity (referred to as “GHRHR^{low} cells”). In certain embodiments, less than about 15% (e.g., less than about 10%, less than about 9%, less than about 8%, less than about 7%, less than about 6%, less than about 5%, less than about 3%, less than about 2%, e.g., about 1%) of the cells of the third cell population express a high level of GHRHR immunoreactivity (referred to as “GHRHR high cells”).

In certain embodiments, the Pit1⁺ cells are or the first cell population is contacted with the one or more GH inducer for at least about 5 days (e.g., about 5 days or about 6 days) to obtain the third cell population. In certain embodiments, the Pit1⁺ cells are or the first cell population is contacted with the one or more GH inducer for at least about 15 days (e.g., about 14 days or about 15 days) to obtain the third cell population.

In certain embodiments, the method comprises contacting the Pit1⁺ cells or the first cell population with RA, corticosteroid, thyroid hormone, a GHRH signaling pathway agonist (e.g., GHRH) for at least about 15 days (e.g., about 14 days or about 15 days) to obtain the third cell population. In certain embodiments, the method comprises contacting the Pit1⁺ cells or the first cell population with RA, corticosteroid, thyroid hormone, two GHRH signaling pathway agonists (e.g., GHRH and cAMP), and an ER agonist (e.g., DPN) for at least about 15 days (e.g., about 14 days or about 15 days) to obtain the third cell population. In certain embodiments, the method comprises contacting the Pit1⁺ cells or the first cell population with RA, corticosteroid, thyroid hormone, a GHRH signaling pathway agonists (e.g., GHRH), a Ghrelin signaling pathway agonist (e.g., Ghrelin), and an ER agonist (e.g., DPN) for at least about 5 days (e.g., about 5 days or about 6 days) to obtain the third cell population.

Extended exposure/contact of the cells to the one or more GH inducer can promote GH proliferation and/or GH maturation. In certain embodiments, the method comprises contacting/exposing the third cell population with/to one or more GH inducer to obtain a fourth cell population comprising at least about 50% (e.g., at least about 60%, at least about 70%, at least about 80%, at least about 90%, at least about 95%, or at least about 99%) GHRHR^{high} cells. In certain embodiments, the fourth cell population comprises less than about 15% (e.g., less than about 10%, less than about 9%, less than about 8%, less than about 7%, less than about 6%, less than about 5%, less than about 3%, less than about 2%, e.g., about 1%) GHRHR^{low} cells.

In certain embodiments, the fourth cell population is obtained at least about 14 days (e.g., about 14 days or 15 days) from the initial contact of the Pit1⁺ cells or the first cell population with the one or more GH inducer. In certain embodiments, the fourth cell population is obtained at least about 9 days (e.g., about 9 days or 10 days) from the initial presence of the third cell population. In certain embodiments, the fourth cell population is obtained at least about 4 weeks (e.g., about 4 weeks, about 5 weeks, or about 6 weeks) from the initial contact of the Pit1⁺ cells or the first cell population with the one or more GH inducer. In certain embodiments, the fourth cell population is obtained at least about two weeks (e.g., about 2 weeks, about 3 weeks or about 4 weeks) from the initial presence of the third cell population.

GHRHR^{high} cells exhibit polygonal morphology with lower proliferating rate, which indicates that they are more mature GH cells (somatotrophs). In addition, GHRHR^{high} cells secrete higher amount of GH (e.g., GH1).

In certain embodiments, greater than about 50%, about 60%, about 70%, about 80%, or about 90% of the population of cells (e.g., at about 5 days, about 6 days, at about 15 days, about 2 weeks, about 19 days, about 20 days, about 4 weeks, or about 6 weeks) from the initial contact of the cells with the one or more GH inducer express detectable levels of the one or more somatotroph marker. In certain embodiments, greater than about 70% of the population of cells express detectable levels of the one or more somatotroph marker. In certain embodiments, the cell population comprises at least about 50% (e.g., about 70% or 80%) of cells expressing one or more somatotroph marker at least about 30 days from the initial contact of the stem cells with the one or more inhibitor of TGFβ/Activin-Nodal signaling. In certain embodiments, the cell population comprises at least about 50% (e.g., about 70% or 80%) of cells expressing one or more somatotroph marker at least about 25 days from the initial contact of the stem cells with the one or more inhibitor of TGFβ/Activin-Nodal signaling.

In certain embodiments, the method comprises contacting the Pit1⁺ cells or the first cell population with the one or more GH inducer (e.g., 1 μM RA, 10 nM T3, 1 M dexamethasone, 1 μM GHRH, and optionally 100 μM c-AMP, and 0.1 nM DPN) for about 14 days (e.g., about 14 or 15 days) to obtain the third cell population. In certain embodiments, the method comprises contacting the Pit1⁺ cells or the first cell population with the one or more GH inducer (e.g., 1 μM RA, 10 nM T3, 1 μM dexamethasone, 1 μM GHRH, and optionally 100 μM c-AMP, and 0.1 nM DPN) for about 2 weeks to obtain the third cell population. In certain embodiments, the method comprises contacting the Pit1⁺ cells or the first cell population with the one or more GH inducer (e.g., 1 μM RA, 10 nM T3, 1 μM dexamethasone, 1 μM GHRH, and optionally 100 μM c-AMP, and 0.1 nM DPN) for about 4 weeks to obtain the fourth cell population. In certain embodiments, the method comprises contacting the Pit1⁺ cells or the first cell population with the one or more GH inducer (e.g., 1 μM RA, 10 nM T3, 1 μM dexamethasone, 1 μM GHRH, and optionally 100 μM c-AMP, and 0.1 nM DPN) for about 6 weeks to obtain the fourth cell population.

In certain embodiments, the method comprises contacting the Pit1⁺ cells or the first cell population with the one or more GH inducer (e.g., 1 μM RA, 10 nM T3, 1 μM dexamethasone, 1 μM GHRH, 10 nM Ghrelin, and 0.1 nM DPN) for about 5 days (e.g., about 5 or 6 days). In certain embodiments, the cells (Pit1⁺ cells) are contacted with the one or more GH inducer (e.g., 1 μM RA, 10 nM T3, 1 μM dexamethasone, 1 μM GHRH, 10 nM Ghrelin, and 0.1 nM DPN) for about 5 days or about 6 days to obtain the third cell population.

In certain embodiments, the method comprises contacting the third cell population with the one or more GH inducer (10 nM T3, 1 μM dexamethasone, 1 μM GHRH, 10 nM Ghrelin, 0.1 nM DPN, and 25 ng/ml IL-6) for about 9 days or about 10 days to obtain the fourth cell population.

In certain embodiments, the method comprises subjecting the fourth cell population to conditions for GH cell maturation. In certain embodiments, the conditions for GH cell maturation comprise exposing the four cell population to the one or more GH inducer (10 nM T3, 1 μM dexamethasone, and 0.1 nM DPN) for at least about 3 days, at least about 4 days, or at least about 5 days. In certain embodiments, the conditions for GH cell maturation comprising exposing the four cell population to the one or more GH inducer (10 nM T3, 1 μM dexamethasone, and 0.1 nM DPN) for about 5 or 6 days.

In certain embodiments, the initial contact of the one or more GH inducer with the Pit1⁺ cells or the first cell population is at least about 15 days and/or no later than about 25 days (e.g., about 15 days, about 16 days, about 17 days, about 18 days, about 19 days or about 20 days) from the initial contact of the stem cells with the one or more inhibitor of TGFβ/Activin-Nodal signaling. In certain embodiments, the Pit1⁺ cells are or the first cell population is contacted with the one or more GH inducer for at least about 14 days or for at least about 2 weeks (e.g., about 14 or 15 days) to obtain the third cell population, and for at least about 4 weeks or for at least about 6 weeks (e.g., for about 4 weeks or for about 6 weeks) to obtain the fourth cell population. In certain embodiments, the Pit1⁺ cells are or the first cell population is contacted with the one or more GH inducer for at least about 5 days (e.g., about 5 days or 6 days) to obtain the third cell population, and for at least about 14 days or at least about two weeks (e.g., about 14 or 15 days) to obtain the fourth cell population.

The presently disclosed subject matter provides in vitro methods for inducing differentiation of stem cells (e.g., human stem cells) into cells expressing one or more somatotroph marker (somatotrophs). In certain embodiments, the method comprises contacting a population of stem cells with (a) an effective amount of one or more inhibitor of transforming growth factor beta (TGFβ)/Activin-Nodal signaling and (b) an effective amount of one or more activator of BMP signaling; and contacting the cells with (c) an effective amount of one or more activator of Sonic Hedgehog (SHH) signaling, (d) an effective amount of one, two or more activators of FGF signaling, (e) an effective amount of one or more dorsalizing agent, (f) an effective amount of one or more ventralizing agent, and (g) an effective amount of one or more Wnt activator.

In certain embodiments, the stem cells are contacted with the one or more activator of BMP signaling for at least about 2 days, or at least about 3 days. In certain embodiments, the stem cells are contacted with the one or more activator of BMP signaling for up to about 3 days, up to about 4 days, or up to about 5 days.

In certain embodiments, the cells are contacted with the one or more activator of SHH signaling and two or more activators of FGF signaling at least about 3 days from the initial contact of the stem cells with the one or more inhibitor of TGFβ/Activin-Nodal signaling. In certain embodiments, the cells are contacted with the one or more activator of SHH signaling and two or more activators of FGF signaling about 3 days or about 4 days from the initial contact of the stem cells with the one or more inhibitor of TGFβ/Activin-Nodal signaling. In certain embodiments, the cells are contacted for at least about 5 days and up to about 10 days with the one or more SHH activator and the two or more FGF activators. In certain embodiments, the cells are contacted for at least about 5 days with the one or more SHH activator, and the two or more FGF activators. In certain embodiments, the cells are contacted for about 5 days or about 6 days with the one or more SHH activator and the two or more FGF activators. In certain embodiments, the cells are contacted for at least about 9 days with the one or more SHH activator and the two or more FGF activators. In certain embodiments, the one or more activator of SHH signaling and two or more activators of FGF signaling are contacted with the cells concurrently.

In certain embodiments, the cells are contacted with the one or more dorsalizing agent, the one or more ventralizing agent, and the one or more Wnt activator for at least about 5 days and up to about 10 days. In certain embodiments, the cells are contacted with the one or more dorsalizing agent, the one or more ventralizing agent, and the one or more Wnt activator for about 7 days. In certain embodiments, the cells are contacted with the one or more dorsalizing agent, the one or more ventralizing agent, and the one or more Wnt activator for about 8 days. In certain embodiments, the cells are contacted with the one or more dorsalizing agent, the one or more ventralizing agent, and the one or more Wnt activator at least about 5 days and up to about 15 days from the initial contact of the stem cells with the one or more inhibitor of TGFβ/Activin-Nodal signaling. In certain embodiments, the cells are contacted with the one or more dorsalizing agent, the one or more ventralizing agent, and the one or more Wnt activator about 8 days from the initial contact of the stem cells with the one or more inhibitor of TGFβ/Activin-Nodal signaling. In certain embodiments, the cells are contacted with the one or more dorsalizing agent, the one or more ventralizing agent, and the one or more Wnt activator about 9 days from the initial contact of the stem cells with the one or more inhibitor of TGFβ/Activin-Nodal signaling. In certain embodiments, the one or more dorsalizing agent, the one or more ventralizing agent, and the one or more Wnt activator are contacted with the cells concurrently.

In certain embodiments, the cells (e.g., pituitary progenitors) are contacted with the one or more dorsalizing agent and the one or more inhibitor of TGFβ/Activin-Nodal signaling for at least about 2 days and up to about 10 days. In certain embodiments, the cells (e.g., pituitary progenitors) are contacted with the one or more dorsalizing agent and the one or more inhibitor of TGFβ/Activin-Nodal signaling for about 4 days. In certain embodiments, the cells (e.g., pituitary progenitors) are contacted with the one or more dorsalizing agent and the one or more inhibitor of TGFβ/Activin-Nodal signaling for about 5 days. In certain embodiments, the cells (e.g., pituitary progenitors) are contacted with the one or more ventralizing agent for at least 2 days and up to about 15 days. In certain embodiments, the cells (e.g., pituitary progenitors) are contacted with the one or more ventralizing agent for at least about 10 days. In certain embodiments, the cells (e.g., pituitary progenitors) are contacted with the one or more ventralizing agent for about 11 days. In certain embodiments, the cells (e.g., pituitary progenitors) are contacted with the one or more ventralizing agent for about 12 days.

In certain embodiments, the initial contact of the cells (e.g., pituitary progenitors) with the one or more dorsalizing agent, the one or more ventralizing agent, and the one or more inhibitor of TGFβ/Activin-Nodal signaling is at least about 5 days and no later than about 15 days from the initial contact of the stem cells with the one or more inhibitor of TGFβ/Activin-Nodal signaling. In certain embodiments, the initial contact of the cells (e.g., pituitary progenitors) with the one or more dorsalizing agent, the one or more ventralizing agent, and the one or more inhibitor of TGFβ/Activin-Nodal signaling is about 8 days from the initial contact of the stem cells with the one or more inhibitor of TGFβ/Activin-Nodal signaling. In certain embodiments, the initial contact of the cells (e.g., pituitary progenitors) with the one or more dorsalizing agent, the one or more ventralizing agent, and the one or more inhibitor of TGFβ/Activin-Nodal signaling is about 9 days from the initial contact of the stem cells with the one or more inhibitor of TGFβ/Activin-Nodal signaling. In certain embodiments, the one or more dorsalizing agent and the one or more inhibitor of TGFβ/Activin-Nodal signaling are contacted with the cells (e.g., pituitary progenitors) concurrently.

In certain embodiments, the cells are further contacted with the one or more Wnt activator and the one or more ER agonist for at least about 5 days and up to about 10 days. In certain embodiments, the cells are further contacted with the the one or more Wnt activator and the one or more ER agonist for about 7 days. In certain embodiments, the cells are contacted with the the one or more Wnt activator and the one or more ER agonist for about 8 days. In certain embodiments, the initial contact of the cells with the one or more Wnt activator and the one or more ER agonist is at least about 2 days and no later than 10 days from the initial contact of the cells (e.g., pituitary progenitors) with the one or more dorsalizing agent and/or at least about 5 days and no later than 25 days from the initial contact of the stem cells with the one or more inhibitor of TGFβ/Activin-Nodal signaling. In certain embodiments, the initial contact of the cells with the one or more Wnt activator and the one or more ER agonist is about 4 day or 5 days from the initial contact of the cells (e.g., pituitary progenitors) with the one or more dorsalizing agent and/or about 12 days or 13 days from the initial contact of the stem cells with the one or more inhibitor of TGFβ/Activin-Nodal signaling. In certain embodiments, the one or more Wnt activator and the one or more ER agonist are contacted with the cells concurrently.

In certain embodiments, the methods further comprise contacting the cells with (h) an effective amount of one or more SMAD inhibitor. In certain embodiments, the one or more SMAD inhibitor is contacted with the cells concurrently with the one or more activator of SHH signaling and two or more activators of FGF signaling.

In certain embodiments, the methods further comprise contacting the cells with (i) an effective amount of one or more ER agonist. In certain embodiments, the one or more ER agonist is contacted with the cells concurrently with the one or more dorsalizing agent and the one or more Wnt activator.

In certain embodiments, the initial contact of the cells with the one or more GH inducer is at least about 10 days and no later than about 25 days from the initial contact of the stem cells with the one or more inhibitor of TGFβ/Activin-Nodal signaling. In certain embodiments, the initial contact of the cells with the one or more GH inducer is at least about 15 days from the initial contact of the stem cells with the one or more inhibitor of TGFβ/Activin-Nodal signaling. In certain embodiments, the initial contact of the cells with the one or more GH inducer is about 15 days from the initial contact of the stem cells with the one or more inhibitor of TGFβ/Activin-Nodal signaling. In certain embodiments, the initial contact of the cells with the one or more GH inducer is about 16 days from the initial contact of the stem cells with the one or more inhibitor of TGFβ/Activin-Nodal signaling. In certain embodiments, the initial contact of the cells with the one or more GH inducer is about 19 days from the initial contact of the stem cells with the one or more inhibitor of TGFβ/Activin-Nodal signaling. In certain embodiments, the initial contact of the cells with the one or more GH inducer is about 20 days from the initial contact of the stem cells with the one or more inhibitor of TGFβ/Activin-Nodal signaling. In certain embodiments, the cells are contacted with the one or more GH inducer for at least about 10 days and up to about 10 weeks. In certain embodiments, the cells are contacted with the one or more GH inducer for at least about 2 weeks to obtain the third cell population and for at least about 4 weeks or at least about 6 weeks to obtain the fourth cell population. In certain embodiments, the cells are contacted with the one or more GH inducer for about 2 weeks to obtain the third cell population. In certain embodiments, the cells are contacted with the one or more GH inducer for about 4 weeks to obtain the fourth cell population. In certain embodiments, the cells are contacted with the one or more GH inducer for about 6 weeks to obtain the fourth cell population.

In certain embodiments, the cells are contacted with the one or more GH inducer for at least about 3 days and up to about 10 weeks. In certain embodiments, the cells are contacted with the one or more GH inducer for at least about 5 days or 6 days to obtain the third cell population, and for at least about 2 weeks to obtain the fourth cell population.

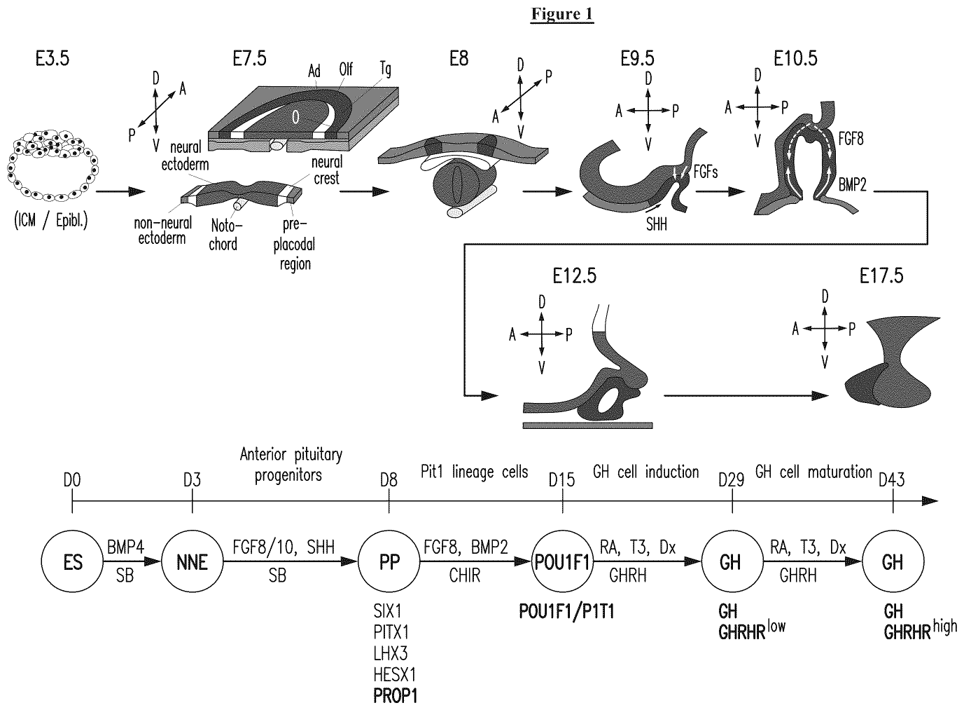

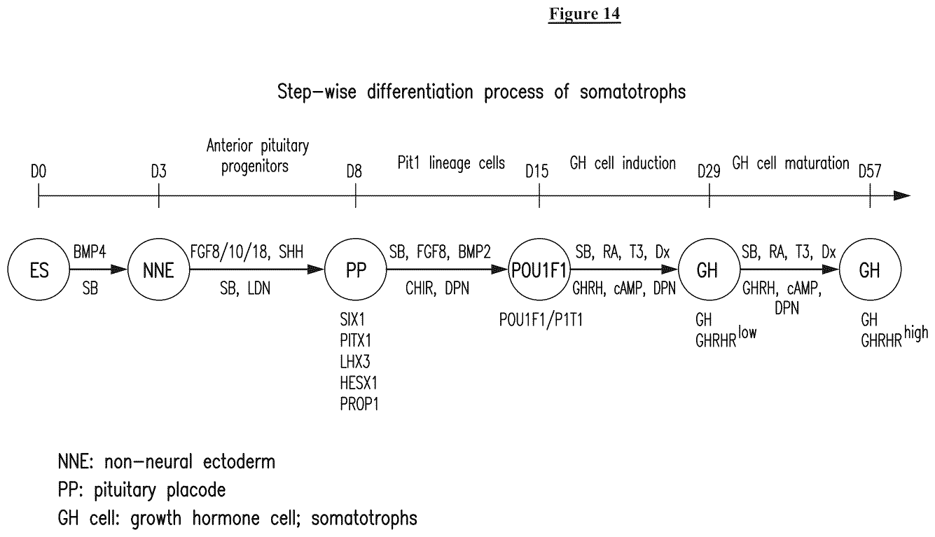

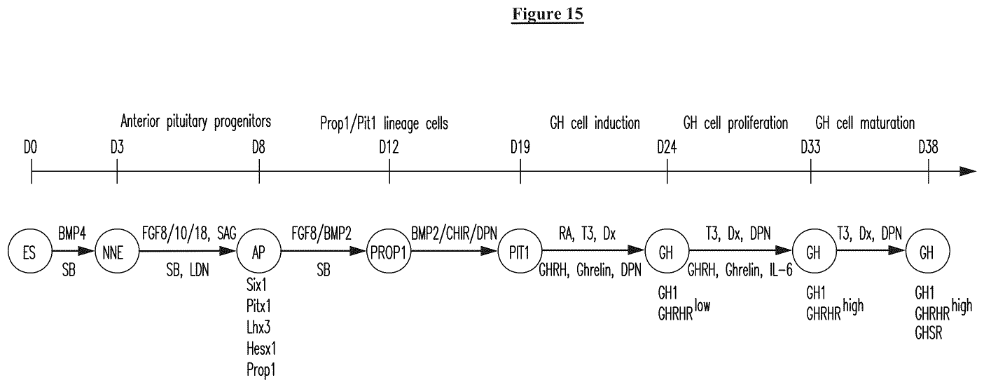

Methods for differentiating stem cells to somatotrophs in accordance with certain embodiments of the presently disclosed subject matter are shown in FIG. 1. Methods for differentiating stem cells to somatotrophs in accordance with certain embodiments of the presently disclosed subject matter are shown in FIG. 14. Methods for differentiating stem cells to somatotrophs in accordance with certain embodiments of the presently disclosed subject matter are shown in FIG. 15.

In certain embodiments, the above-described inhibitors, activators and inducers are added to a cell culture medium comprising the cells being differentiated. Suitable cell culture media include, but are not limited to, Essential 8®/Essential 6® (“E8/E6”) medium. E8/E6 medium is commercially available.

E8/E6 medium is a feeder-free and xeno-free medium that supports the growth and expansion of human pluripotent stem cells. E8/E6 medium has been proven to support somatic cell reprogramming. In addition, E8/E6 medium can be used as a base for the formulation of custom media for the culture of PSCs. One example E8/E6 medium is described in Chen et al., Nat Methods. 2011 May; 8 (5): 424-9, which is incorporated by reference in its entirety. One example E8/E6 medium is disclosed in WO15/077648, which is incorporated by reference in its entirety. In certain embodiments, an E8/E6 cell culture medium comprises DMEM/F12, ascorbic acid, selenium, insulin, NaHCO₃, transferrin, FGF2 and TGFβ. In certain embodiments, the E6 media does not include FGF2 and TGFβ. The E8/E6 medium differs from a KSR medium in that E8/E6 medium does not include an active BMP or Wnt ingredient.

The differentiated cells can further express one or more reporter. Non-limiting examples of reporters include fluorescent proteins (such as green fluorescent protein (GFP), blue fluorescent protein (EBFP, EBFP2, Azurite, mKalama1), cyan fluorescent protein (ECFP, Cerulean, CyPet, mTurquoise2), and yellow fluorescent protein derivatives (YFP, Citrine, Venus, YPet, EYFP)), β-galactosidase (LacZ), chloramphenicol acetyltransferase (cat), neomycin phosphotransferase (neo), enzymes (such as oxidases and peroxidases), and antigenic molecules. As used herein, the terms “reporter gene” or “reporter construct” refer to genetic constructs comprising a nucleic acid encoding a protein that is easily detectable or easily assayable, such as a colored protein, fluorescent protein such as GFP or an enzyme such as beta-galactosidase (lacZ gene). In certain embodiments, the reporter can be driven by a recombinant promotor of a NE lineage marker gene, a recombinant promotor of a NC lineage marker gene, a recombinant promotor of a CP lineage marker gene, or a recombinant promotor of a NNE lineage marker gene.

The differentiated cells can be purified after differentiation, e.g., in a cell culture medium. As used herein, the terms “purified,” “purify,” “purification,” “isolated,” “isolate,” and “isolation” refer to the reduction in the amount of at least one contaminant from a sample. For example, a desired cell type is purified by at least about 10%, by at least about 30%, by at least about 50%, by at least about 75%, by at least about 80%, by at least about 85%, by at least about 90%, or by at least about 90%, with a corresponding reduction in the amount of undesirable cell types. The term “purify” can refer to the removal of certain cells (e.g., undesirable cells) from a sample.

The presently disclosed subject matter also provides a population of in vitro differentiated cells expressing one or more pituitary precursor/progenitor marker, differentiated cells expressing Pit1, and/or differentiated cells expressing one or more somatotroph marker (including GHRHR^{low} cells and GHRHR^{high} cells), produced by the methods described herein, and compositions comprising such in vitro differentiated cells.

### 5.3 Compositions Comprising Differentiated Cell Populations

The presently disclosed subject matter provides compositions comprising a population of cells expressing one or more pituitary precursor/progenitor marker produced by the in vitro differentiation methods described herewith. Furthermore, the presently disclosed subject matter provides compositions comprising a population of cells expressing Pit1 produced by the in vitro differentiation methods described herewith. In addition, the presently disclosed subject matter provides compositions comprising a population of GH-producing somatotrophs (including GHRHR low cells and GHRHR^{high} cells) produced by the in vitro differentiation methods described herewith.

Furthermore, the presently disclosed subject matter provides compositions comprising a population of in vitro differentiated cells, wherein at least about 50% (e.g., at least about 55%, at least about 60%, at least about 65%, at least about 70%, at least about 75%, at least about 80%, at least about 85%, at least about 90%, at least about 95%, at least about 99%, or at least about 99.5%) of the population of differentiated cells express one or more pituitary precursor/progenitor marker, and wherein less than about 25% (e.g., less than about 20%, less than about 15%, less than about 10%, less than about 5%, less than about 4%, less than about 3%, less than about 2%, less than about 1%, less than about 0.5%, or less than about 0.1%) of the population of differentiated cells express one or more marker selected from the group consisting of stem cell markers, non-neural ectoderm (NNC) markers, neural crest (NC) lineage markers, and non-pituitary placode markers (including, but not limited to, cranial placode markers, epibranchial placode markers, trigeminal placode markers, and otic placode markers).

Furthermore, the presently disclosed subject matter provides compositions comprising a population of in vitro differentiated cells, wherein at least about 50% (e.g., at least about 55%, at least about 60%, at least about 65%, at least about 70%, at least about 75%, at least about 80%, at least about 85%, at least about 90%, at least about 95%, at least about 99%, or at least about 99.5%) of the population of differentiated cells express Pit1, and wherein less than about 25% (e.g., less than about 20%, less than about 15%, less than about 10%, less than about 5%, less than about 4%, less than about 3%, less than about 2%, less than about 1%, less than about 0.5%, or less than about 0.1%) of the population of differentiated cells express one or more marker selected from the group consisting of pituitary precursor markers, stem cell markers, NNE markers, neural crest (NC) lineage markers, and non-pituitary placode markers (including, but not limited to, cranial placode markers, epibranchial placode markers, trigeminal placode markers, and otic placode markers). In certain embodiments, such cell population is the first cell population disclosed in Section 5.2.2.

Furthermore, the presently disclosed subject matter provides compositions comprising a population of in vitro differentiated cells, wherein at least about 50% (e.g., at least about 55%, at least about 60%, at least about 65%, at least about 70%, at least about 75%, at least about 80%, at least about 85%, at least about 90%, at least about 95%, at least about 99%, or at least about 99.5%) of the population of differentiated cells are GH-producing somatotrophs, and wherein less than about 25% (e.g., less than about 20%, less than about 15%, less than about 10%, less than about 5%, less than about 4%, less than about 3%, less than about 2%, less than about 1%, less than about 0.5%, or less than about 0.1%) of the population of differentiated cells express one or more marker selected from the group consisting of lactrotroph markers, thyrotroph markers, Pit1, pituitary precursor markers, stem cell markers, NNE markers, neural crest (NC) lineage markers, and non-pituitary placode markers (including, but not limited to, cranial placode markers, epibranchial placode markers, trigeminal placode markers, and otic placode markers). In certain embodiments, such cell population includes the third cell population disclosed in Section 5.2.2, the fourth cell population disclosed in Section 5.2.2, and a combination thereof.

Non-limiting examples of stem cell markers include OCT4, NANOG, SOX2, LIN28, SSEA4, and SSEA3.

Non-limiting examples of NC lineage markers include SOX10, FoxD3, ASCL1, Neurogenin, and Snail.

Non-limiting examples of non-pituitary placode markers include cranial placode markers (including, but not limited to, SIX1, PAX6, PITX3, Crystallin alpha A, and crystallin alpha B), epibranchial placode markers (including, but not limited to, PAX2), trigeminal placode markers (including, but not limited to, PAX3), otic placode markers (including, but not limited to, PAX8).

Non-limiting examples of NNE markers include TFAP2A, EYA1, DLX3, and DLX5.

Non-limiting examples of pituitary progenitor markers include SIX1, LHX3, LHX4, PITX1, PITX2, HESX1, PROP1, SIX6, TBX19, PAX6, GATA2, and SF1.

Non-limiting examples of somatotroph markers include GH, GHRHR, POU1F1, NeuroD4, and GHSR.

Non-limiting examples of lactrotroph markers include PRL, PIT1, and D2R.

Non-limiting examples of thyrotroph markers include TSH, THRH, and PIT1.

In certain embodiments, the composition comprises a population of from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of the presently disclosed stem-cell-derived cells.

In certain embodiments, the composition comprises a population of from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of the presently disclosed in vitro-differentiated somatotroph cells.

In certain embodiments, said composition is frozen. In certain embodiments, said composition may further comprise one or more cryoprotectant, for example, but not limited to, dimethylsulfoxide (DMSO), glycerol, polyethylene glycol, sucrose, trehalose, dextrose, or a combination thereof.

In certain non-limiting embodiments, the composition further comprises a biocompatible scaffold or matrix, for example, a biocompatible three-dimensional scaffold that facilitates tissue regeneration when the cells are implanted or grafted to a subject. In certain non-limiting embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties).

In certain embodiments, the composition is a pharmaceutical composition that comprises a pharmaceutically acceptable carrier, excipient, diluent or a combination thereof. The compositions can be used for preventing and/or treating GH deficiency, as described herein.

The presently disclosed subject matter also provides a device comprising the differentiated cells or the composition comprising thereof, as disclosed herein. Non-limiting examples of devices include syringes, fine glass tubes, stereotactic needles and cannulas.

### 5.4 Method of Increasing Growth Hormone

The in vitro differentiated cells that express one or more somatotroph marker (also referred to as “stem-cell-derived somatotrophs”, e.g., GH-producing somatotrophs) can be used for increasing GH expression and/or secretion, and/or restoring dynamic release of one or more of GH, Insulin-like growth factor 1 (IGF-1), and IGF-2 (e.g., in a physiological manner). Thus, the stem-cell-derived somatotrophs can be used for treating GH deficiency.

Hormonal deficiency is currently a treatable condition, but GH replacement is incredibly costly and challenging in the young patient, who requires daily (sometimes twice a day) injections 6-7 times a week at a significant expense not fully covered by insurance and often exceeding $50,000 a year. Thus, treatment is often interrupted at puberty though there is data in support of continued requirements for GH in young adulthood for restoration of metabolism and maintenance of bone mass. Since the manufacture of GH as a recombinant product, there has not been any innovation in the field.

The presently disclosed subject matter provides methods of increasing GH expression and/or secretion, restoring dynamic release of one or more of GH, Insulin-like growth factor 1 (IGF-1), and IGF-2, and/or treating GH deficiency in a subject, comprising administering to the subject an effective amount of one or more of the followings:
(a) a population of stem-cell-derived somatotrophs described herein;
(b) a composition comprising such stem-cell-derived somatotrophs; and
(c) a device comprising such composition.

Furthermore, the presently disclosed subject matter provides for uses of the presently disclosed stem-cell-derived somatotrophs or a composition comprising thereof or a device comprising thereof for increasing GH expression and/or secretion, and/or restoring one or more of GH, Insulin-like growth factor 1 (IGF-1), and IGF-2.

Growth hormone deficiency includes congenital growth hormone deficiency and acquired growth hormone deficiency. Congenital growth hormone deficiency is due to mutations of genes involved in growth hormone development. Acquired growth hormone deficiency can be induced by tumor, surgery, injuries in the hypothalamic-pituitary region, etc. Congenital growth hormone deficiency can be divided into two categories: combined pituitary hormone deficiency (CPHD) and isolated growth hormone deficiency (IGHD) Types. Genetic mutations that can cause CPHD include POU1F1 mutation (CPHD1), PROP-1 mutation (CPHD2; most common, 12-55%), LHX3 mutation (CPHD3), and LHX4 mutation (CPHD4). Genetic mutations that can cause IGHD types include GH1 mutation (Types IA and II), GH1 or Growth-hormone-releasing hormone receptor (GHRHR) mutation (Type IB), and Bruton tyrosine kinase (BTK) mutation (Type III).

In certain embodiments, the subject suffers from a GH deficiency. The GH deficiency can be genetic (e.g., dwarfism), related to trauma, tumor, surgery, and radiation, e.g., related to pituitary lesion and/or pituitary suppression caused by a medical treatment. In certain embodiments, the subject suffers from dwarfism. Non-limiting examples of GH deficiency include dwarfism, osteoporosis, decrease in muscle mass, decrease in bone mass, mental disorders such as depression or anxiety, weakening of the immune system or alterations in metabolism. In certain embodiments, the GH deficiency is dwarfism.

In certain embodiments, the presently disclosed stem-cell-derived somatotrophs are directly injected into an organ of interest (e.g., an organ affected by GH deficiency, e.g., pituitary gland (e.g., anterior pituitary gland, the hypothalamus or median eminence, the subcutaneous tissue). In certain embodiments, the presently disclosed stem-cell-derived somatotrophs, a composition or a device comprising thereof are administered (injected) directly to a subject's pituitary gland (e.g., anterior pituitary gland, the hypothalamus or median eminence, the subcutaneous tissue), e.g., by a transnasal and/or transphenoidal approach. In certain embodiments, the presently disclosed stem-cell derived somatotrophs can be encapsulated in a polymer and injected subcutaneously.

The presently disclosed stem-cell-derived somatotrophs can be administered in any physiologically acceptable vehicle. Pharmaceutical compositions comprising the presently disclosed stem-cell-derived somatotrophs and a pharmaceutically acceptable carrier are also provided. The presently disclosed stem-cell-derived somatotrophs and the pharmaceutical compositions comprising thereof can be administered via localized injection, orthotropic (OT) injection, systemic injection, intravenous administration, subcutaneous administration, or parenteral administration.

The presently disclosed stem-cell-derived somatotrophs or compositions comprising thereof can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, e.g., a composition comprising the presently disclosed stem-cell-derived somatotrophs, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the presently disclosed subject matter, however, any vehicle, diluent, or additive used would have to be compatible with the presently disclosed stem-cell-derived somatotrophs.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the presently disclosed stem-cell-derived enteric NC precursors. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the presently disclosed stem-cell-derived somatotrophs is the quantity of cells necessary to achieve an optimal effect. An optimal effect includes, but are not limited to, increased GH expression, increased GH secretion, restoration of dynamic release of one or more of GH, Insulin-like growth factor 1 (IGF-1), and IGF-2, and/or restoration of growth and normalization of metabolism.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to increase the expression and/or secretion of GH; restore the dynamic release of one or more of GH, Insulin-like growth factor 1 (IGF-1), and IGF-2, restoration of growth and normalization of metabolism, and/or palliate, ameliorate, stabilize, reverse or slow the progression of a GH deficiency (e.g., dwarfism), or otherwise reduce the pathological consequences of a GH deficiency (e.g., dwarfism). The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In certain embodiments, an effective amount is an amount that is sufficient to achieve an optimal effect (including, but not limited to, increased GH expression, increased GH secretion, restoration of dynamic release of one or more of GH, Insulin-like growth factor 1 (IGF-1), and IGF-2, and/or restoration of growth and normalization of metabolism). In certain embodiments, an effective amount is an amount that is sufficient to increase the GH expression in a subject (e.g., one suffering from a GH deficiency) by about 10%, about 20%, about 30%, about 40%, about 50%, about 60%, about 70%, about 80%, about 90%, about 95%, about 98%, about 99% or about 100%. In certain embodiments, an effective amount is an amount that is sufficient to increase the GH secretion in a subject (e.g., one suffering from a GH deficiency) by about 10%, about 20%, about 30%, about 40%, about 50%, about 60%, about 70%, about 80%, about 90%, about 95%, about 98%, about 99% or about 100%. In certain embodiments, an effective amount is an amount that is sufficient to increase the body size of a subject (e.g., one suffering from a GH deficiency) by about 1%, about 5%, about 10%, about 20%, about 30%, about 40%, about 50%, about 60%, about 70%, about 80%, about 90%, about 95%, about 98%, about 99% or about 100%. In certain embodiments, an effective amount is an amount that is sufficient to increase the body weight of a subject (e.g., one suffering from a GH deficiency) by about 1%, about 5%, about 10%, about 20%, about 30%, about 40%, about 50%, about 60%, about 70%, about 80%, about 90%, about 95%, about 98%, about 99% or about 100%.

The quantity of cells to be administered will vary for the subject being treated. In certain embodiments, from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ the presently disclosed stem-cell-derived somatotrophs are administered to a subject. In certain embodiments, from about 1×10⁵ to about 1×10⁷ the presently disclosed stem-cell-derived somatotrophs are administered to a subject suffering from a GH deficiency.

### 5.5 Kits

The presently disclosed subject matter provides for kits for inducing differentiation of stem cells. In certain embodiments, the kit comprises (a) one or more inhibitor of transforming growth factor beta (TGFβ)/Activin-Nodal signaling, (b) one or more activator of BMP signaling, (c) one or more activator of FGF signaling, (d) one or more activator of SHH signaling, and (e) instructions for inducing differentiation of the stem cells into a population of differentiated cells that express one or more pituitary precursor marker. In certain embodiments, the kit further comprises (f) one or more SMAD inhibitor.

In certain embodiments, the kit comprises (a) one or more inhibitor of transforming growth factor beta (TGFβ)/Activin-Nodal signaling, (b) one or more activator of BMP signaling, (c) one or more activator of FGF signaling, (d) one or more activator of SHH signaling, (e) one or more dorsalizing agent, (f) one or more ventralizing agent, (g) one or more Wnt activator, and (h) instructions for inducing differentiation of the stem cells into a population of differentiated cells that express Pit1. In certain embodiments, the kit further comprises (i) one or more ER agonist.

In certain embodiments, the kit comprises (a) one or more inhibitor of transforming growth factor beta (TGFβ)/Activin-Nodal signaling, (b) one or more activator of BMP signaling, (c) one or more activator of FGF signaling, (d) one or more activator of SHH signaling, (e) one or more dorsalizing agent, (f) one or more ventralizing agent, (g) one or more Wnt activator, (h) one or more GH inducer, and (i) instructions for inducing differentiation of the stem cells into a population of GH-producing somatotrophs.

In certain embodiments, the instructions comprise contacting the stem cells with the inhibitor(s), activator(s), agent(s), and inducer(s) in a specific sequence. The sequence of contacting the inhibitor(s), agent(s), and inducer(s) can be determined by the cell culture medium used for culturing the stem cells.

In certain embodiments, the instructions comprise contacting the stem cells with the inhibitor(s), activator(s) agent(s), and inducer(s) as described by the methods of the present disclosure (see, supra, Section 5.2).

In certain embodiments, the present disclosure provides kits comprising an effective amount of a population of the presently disclosed stem-cell-derived somatotrophs or a composition comprising said somatotroph in unit dosage form. In certain embodiments, the stem-cell-derived cells are mature differentiated cells. In certain embodiments, the kit comprises a sterile container which contains the therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit comprises instructions for administering a population of the presently disclosed stem-cell-derived somatotrophs or a composition comprising thereof to a subject (e.g., a subject suffering from a GH deficiency). The instructions can comprise information about the use of the cells or composition for treating GH deficiency. In certain embodiments, the instructions comprise at least one of the following: description of the therapeutic agent; dosage schedule and administration for treating or preventing a neurodegenerative disorder or symptoms thereof; precautions; warnings; indications; counter-indications; over dosage information; adverse reactions; animal pharmacology; clinical studies; and/or references. The instructions can be printed directly on the container (when present), or as a label applied to the container, or as a separate sheet, pamphlet, card, or folder supplied in or with the container.

### 5.6 Methods of Screening Therapeutic Compounds

The presently disclosed stem-cell-derived somatotrophs can be used to model GH deficiency, and can also serve as a platform to screen for candidate compounds that can overcome cellular phenotypes related to GH deficiency. The capacity of a candidate compound to alleviate GH deficiency can be determined by assaying the candidate compound's ability to rescue a physiological or cellular defect, which causes GH deficiency.

In certain embodiments, the method comprises: (a) providing (i) a population of the presently disclosed somatotrophs derived from stem cells (e.g., human stem cells) wherein the somatotrophs are prepared from human stem cells (e.g., human pluripotent stem cells, e.g., hESCs, or hiPSCs) from a subject with GH deficiency, or wherein the somatotrophs express cellular and/or metabolic characteristics of GH deficiency, and (ii) a test compound; (b) contacting the somatotrophs with the test compound; and (c) measuring functional activity, or gene expression of the somatotrophs. In certain embodiments, the somatotrophs are contacted with the test compound for at least about 24 hours (1 day), about 2 days, about 3 days, about 4 days, about 5 days, about 6 days, about 7 days, about 8 days, about 9 days, or about 10 days.

The presently disclosed subject matter will be better understood by reference to the following Examples and Appendix, which are provided as exemplary of the presently disclosed subject matter, and not by way of limitation.

### Example 1—Selective Derivation of Human Somatotrophs From Stem Cells Summary

A method of derivation of somatotrophs from human pluripotent stem cells by monolayer culture is provided. The method resulted in somatotrophs that were able to release growth hormone. The procedure was a step-wise differentiation process which involved exposure to various molecules at different timing to induce a few developmental stages of progenitors/precursors, which gave rise to somatotrophs. Transplantation of the cells to preclinical model for GH deficient mice led to significant increase of GH and IGF-1 levels in plasma.

### Methods and Materials

Human ES or iPS cells were expanded in E8 media on matrigel.

On day 0, cells were changed to E6 media where BMP4 (5 ng/ml) and SB43152 (10 μM) were added, with daily medium and factors change.

On day 3, BMP4 was removed and the following factors added: FGF8 (100 ng/ml); FGF10 (50 ng/ml); FGF18 (50 ng/ml); Sonic hedgehog (SHH) (100 ng/ml) or SAG (Smoothened Agonist) (100 nM); LDN193189 (250 nM). Media and factors were changed every other day.

On day 8, the following factors were used: FGF8 (50 ng/ml); BMP2 (20 ng/ml); CHIR 99021 (3 μM); DPN 2,3-bis(4-hydroxyphenyl) propionitrile (0.1 nM). Media and factors are changed every other day.

On day 15, the following factors were used: Retinoic acid (1 μM); Thyroid hormone T3 (10 nM); Dexamethasone (1 μM); cyclic AMP (100 μg/ml); DPN (0.1 nM); Growth Hormone Releasing Hormone (GHRH) (1 μM). Media and factors were changed every other day.

On day 29, the cells entered the maturation phase and could be maintained in the same factors for a variable period ranging from 1-4 weeks. Cells at this stage expressed GHRH receptors (GHRHR). They could be subjected to FACS sorting for GHRH in order to obtain a pure cell population of somatotrophs. Cells remained on matrigel for the duration of the protocol.

See for example, the protocols shown in FIGS. 1 and 14.

This protocol was GMP compatible. These conditions suppressed effectively other pituitary cell lineages such as ACTH secreting corticotrophs, prolactin secreting cells, FSH/LH and TSH secreting cells.

### Results

In order to derive GH cells, the strategy was to first differentiate human pluripotent stem cells to pre-placodal ectoderm and anterior pituitary progenitors. The cells were further differentiated to PROP1 lineage, and then to PIT1 lineage. The cells were then terminally differentiated to GH cells. The step-wise protocol to differentiate pluripotent stem cells into growth hormone secreting cells involved promoting Prop1 expression in anterior pituitary progenitors, activating POU1F1 (PIT1), directing cell differentiation to growth hormone expressing cells. The growth hormone expressing cells were also further matured via prolonged incubation. The key markers to monitor the process of differentiation comprised Prop1, POU1F1 (PIT1), GH and GHRHR.

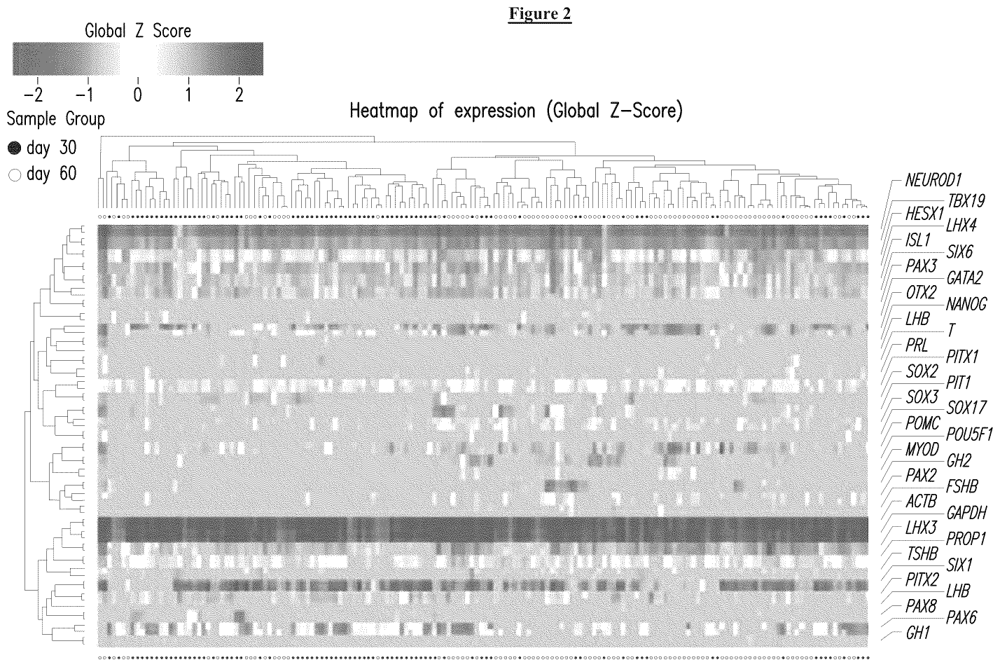

For example, the first step was to induce non-neural ectoderm by BMP4 and inhibitor of TGF-β/Activin/NODAL pathway (e.g., SB431542). Then the cells were subjected to inhibitor of TGF-β/Activin/NODAL pathway (e.g., SB431542), FGF8/10/18, SHH agonist (e.g., SHH), and a SMAD inhibitor (e.g., LDN 193189) to induce anterior pituitary progenitors, which were characterized by PITX1/2, LHX3/4 and PROP1 expression. Gene expressions of the resulting pre-placodal ectoderm and anterior pituitary progenitors were analyzed by single cell RNA-seq, and the results are shown in FIG. 2. 70% of cells co-expressed pituitary transcripts such as PITX1 and LHX3. Only four cells (5% of all cells analyzed) expressed T (mesoderm), SOX17, or MYOD, suggesting a low percentage of contaminating cells. The other placode fates included PAX2 (epibranchial), PAX3 (trigeminal), or PAX8 (otic), which together were detected in about 20% of the SIX1+ population.

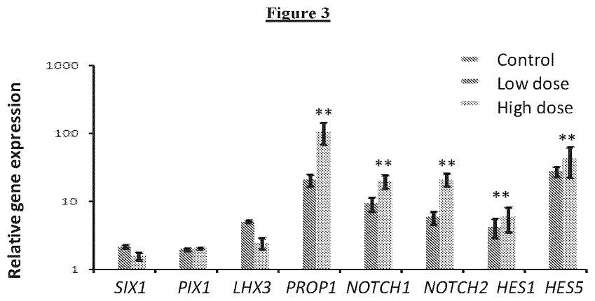

Additionally, Notch signaling inducers (e.g., BMP4, SB431542, FGF8/10 and SHH) were able to increase PROP1 expression. FIG. 3 shows that cells treated with Notch signaling inducers exhibited increased PROP1 expression.

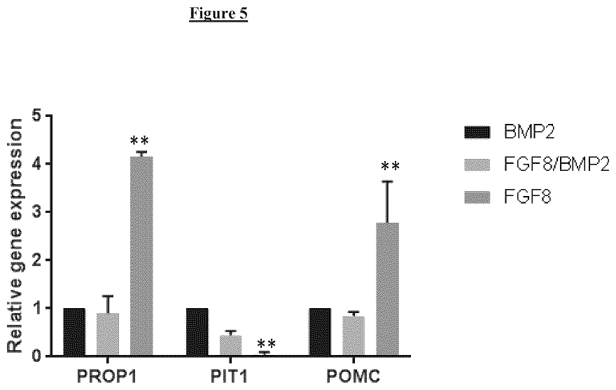

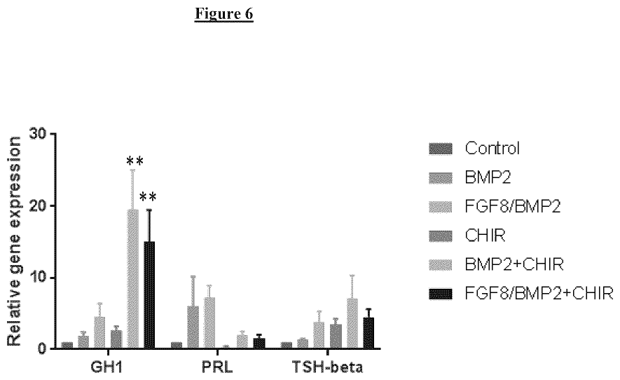

The cells were then treated with TGF-β/Activin/NODAL pathway (e.g., SB431542), WNT pathway activator (e.g., CHIR 99021), FGF8, BMP2 and estrogen ERB receptor selective agonist (e.g., DPN) to induce PIT1 (POU1F1) expressing cells which were able to give rise to somatotrophs, lactrotrophs and Thyrotrophs. CHIR99021, which is GSK-β inhibitor to upregulate Wnt/β-catenin signaling were able to induce Pit1 expression. FIG. 4 shows PIT1 gene expression in cells treated by CHIR99021. Most PIT1 cells were not proliferating shown by lack of Ki67 expression. FIG. 5 shows that FGF8 treatment were able to maintain PROP1 expression, however, FGF8 treatment alone downregulated PIT1 and upregulated POMC. Addition of BMP2 were able to counter-balance the effects of FGF8. The effects of the combination of FGF8, BMP2 and CHIR are shown in FIG. 6. The combination significantly promoted GH1 expression.

For selective differentiation to somatotrophs, retinoic acid were used to suppress POMC expressed by corticotrophs and to promote GH1 expression which is expressed by somatotrophs. In addition, the corticosteroid (e.g., Dexamethasone) and thyroid hormone (e.g., T3) were added to the medium to inhibit PRL (expressed by lactotroph) and TSH (expressed by thyrotroph), while promoting GH1 expression. GH1 expression was further promoted by estrogen ERB receptor selective agonist (DPN) and GHRH signaling pathway agonists (e.g., GHRH and Debutyl-cAMP). The effects of RA, GHRH, T3, and corticosterone on selective induction of growth hormone secreting cells are shown in FIG. 6. FIG. 6A shows that treatment with RA significantly increased GH1 expression. FIG. 6B shows that treatment with GHRH significantly increased GH1 expression. FIG. 6C shows that treatment with T3 significantly reduced TSH-β expression. FIG. 6D shows that treatment with corticosterone significantly reduced PRL expression.

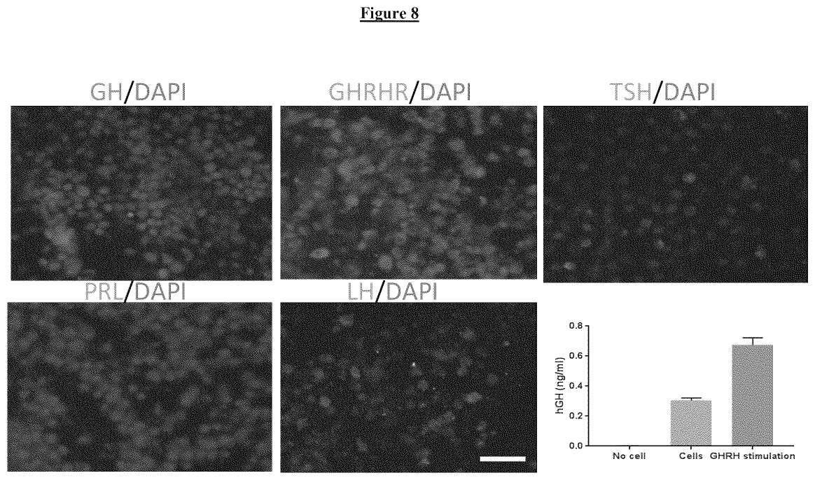

After this step, the majority population in culture were somatotrophs which expressed the somatotroph marker GH1 and secreted growth hormone in the supernatant. The phenotype of the population is shown FIG. 8. The cell population expressed high amount of GH and GHRHR, and low amount of TSH, PRL and LH. GH expression was further increased upon GHRH stimulation (FIG. 8, lower right panel).

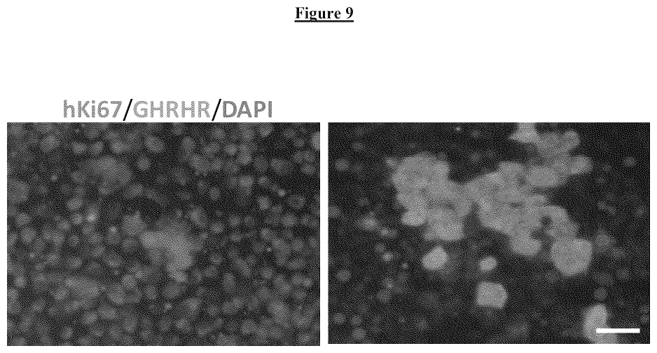

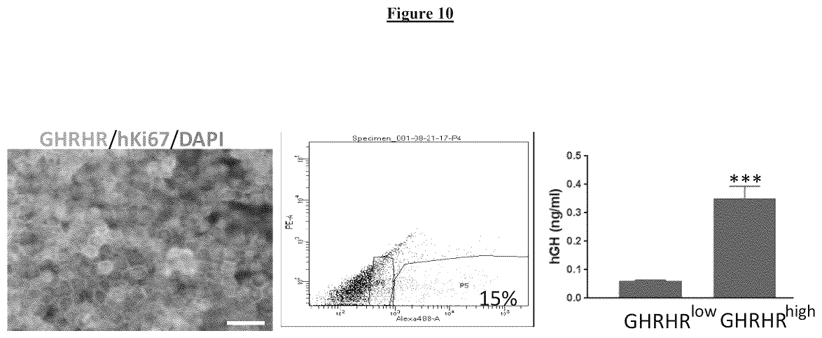

The final step of the method was to further allow maturation of the somatotrophs and increase expression of GHRH over several weeks. Somatotrophs were enriched by cell sorting (e.g., FACS) by anti-GHRHR. FIG. 9 shows that in GHRHR positive cells, most cells expressed low level of GHRHR and only some of the cells expressed high level of GHRHR. The cells expressing high level of GHRHR exhibited polygonal morphology with lower proliferating rate, indicating they were more mature GH cells. FIG. 10 shows that cells expressing high level of GHRHR had low proliferating rate reflected by the low Ki67 expression, and expressed significantly higher amount of growth hormone compared to cells expressing low level. These cells could be further enriched by cell sorting (e.g., FACS) by anti-GHRHR.

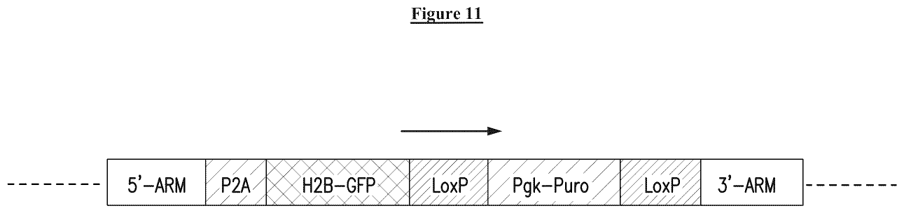

Endogenous reporters inserted by gene editing can be included for closer monitoring of the progress of the differentiation. An exemplary reporter construct for generating stage specific reporter lines is shown in FIG. 11.

### Example 2—Use of Differentiated Cells for Treating GH Deficiency

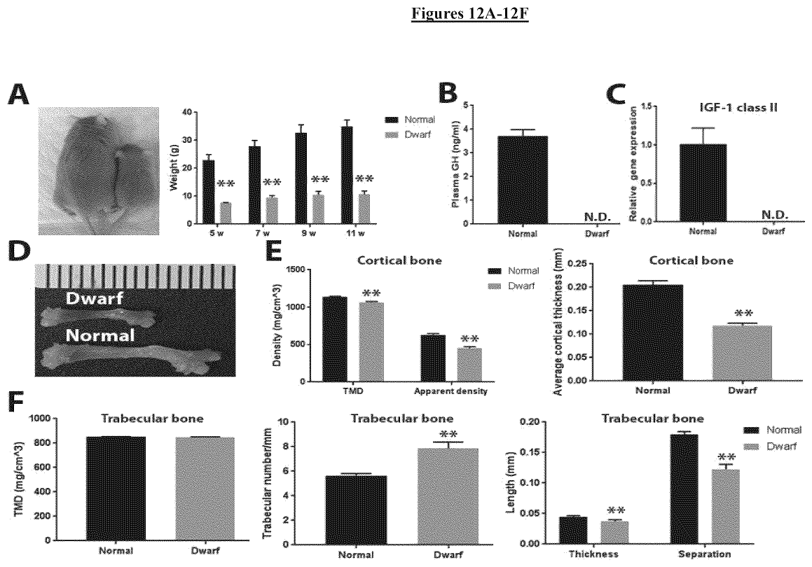

For pre-clinical model for GH deficient mice strains, the Ames dwarf mouse was chosen, as it had PROP1 mutation, which represented the most common CPHD in human. Ames dwarf mice exhibited half of size of normal mice, with undetectable levels of GH and IGF-1 (FIGS. 12A-12C). Micro-CT analysis were performed on femur and the results are shown in FIGS. 12D-12F. The main difference was in cortical bone. Mineral density and average cortical thickness of Ames dwarf mice were significant lower than the normal mice. Additionally, Ames mice showed a much thinner trabecular bone with more separation and a higher trabecular number.

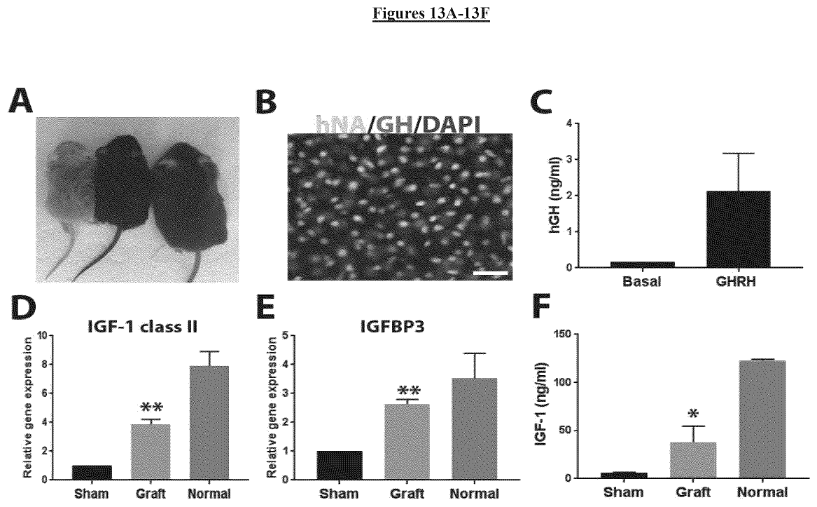

Rag1^{-/-} and Ames dwarf mice were crossbred to obtain immunodeficient Ames dwarf mice to receive human cells. The crossbred Ames dwarf mice still exhibited similar size as Ames dwarf mice, which was about half the size of normal mice (FIG. 13A). GHRHR positive cells obtained according to the methods described in Example 1 were inject subcutaneously to immuno-deficient Ames dwarf mice. The cells survived 6 weeks after graft and express GH (see FIG. 13B). The graft was stimulated by GHRH injection, which led to elevated plasma level of human GH (see FIG. 13C). The circulating forms of IGF-1 and IGFBP3 expression were recovered to 48% and 75% in liver (see FIGS. 13D and 13C). The plasma IGF-1 level was about 31% of normal level (see FIG. 13F). For this experiment, only 150,000 cells were injected. Optimal results can be achieved by increasing the graft cell number.

Thus, these results could provide a much needed preclinical proof of concept that hormone-producing cells can be grafted in the hypothalamic-pituitary interface, can function and integrate the endocrine system, leading to physiologically appropriate function including dynamic release and feedback responsiveness, and rescue of deficits. The results represented herein can result in key insights into a) the feasibility of cell grafting for GH deficiency as a proof of concept, b) the integration of grafted cells into the hypothalamic-pituitary axis and the restoration of dynamic release of GH in a physiological manner, and c) the development of valuable preclinical data under cGMP compliant conditions that will accelerate work towards potential clinical application. Furthermore, the cell differentiation protocol can be produced by prioritizing compounds and methods that are fully compatible with cGMP conditions.

### Example 3—Selective Derivation of Human Somatotrophs From Stem Cells

### Summary

A method of derivation of somatotrophs from human pluripotent stem cells by monolayer culture is provided. The method resulted in somatotrophs that were able to release growth hormone. The procedure was a step-wise differentiation process which involved exposure to various molecules at different timing to induce a few developmental stages of progenitors/precursors, which gave rise to somatotrophs. Transplantation of the cells to preclinical model for GH deficient mice led to significant increase of GH and IGF-1 levels in plasma.

### Methods and Materials

Human ES or iPS cells were expanded in E8 media on matrigel.

On day 0, cells were changed to E6 media where BMP4 (5 ng/ml) and SB43152 (10 μM) were added, with daily medium and factors change.

On day 3, BMP4 was removed and the following factors added: FGF8 (100 ng/ml); FGF10 (50 ng/ml); FGF18 (50 ng/ml); Sonic hedgehog (SHH) (100 ng/ml) or SAG (Smoothened Agonist) (100 nM); LDN193189 (250 nM). Media and factors were changed every other day.

On day 8, the following factors were used: FGF8 (100 ng/ml); BMP2 (20 ng/ml); SB43152 (10 μM). Media and factors are changed every other day.

On day 12, the following factors were used: BMP2 (20 ng/ml); CHIR 99021 (3 μM); DPN 2,3-bis(4-hydroxyphenyl) propionitrile (0.1 nM). Media and factors are changed every other day.

On day 19, the following factors were used: Retinoic acid (0.1 μM); Thyroid hormone T3 (10 nM); Dexamethasone (1 μM); DPN (0.1 nM); Growth Hormone Releasing Hormone (GHRH) (10 nM), Ghrelin (10 nM). Media and factors were changed every other day.

On day 24, the following factors were used: Thyroid hormone T3 (10 nM); Dexamethasone (1 μM); DPN (0.1 nM); Growth Hormone Releasing Hormone (GHRH) (10 nM); Ghrelin (10 nM); IL-6 (25 ng/ml). Media and factors were changed every other day.

On day 33, the following factors were used: Thyroid hormone T3 (10 nM); Dexamethasone (1 μM); DPN (0.1 nM). Media and factors were changed every other day.

On day 33, the cells entered the maturation phase. Cells at this stage expressed GHRH receptors (GHRHR).

See for example, the protocol shown in FIG. 15.

They could be subjected to FACS sorting for GHRH in order to obtain a pure cell population of somatotrophs. Cells remained on matrigel for the duration of the protocol. This protocol was GMP compatible. These conditions suppressed effectively other pituitary cell lineages such as ACTH secreting corticotrophs, prolactin secreting cells, FSH/LH and TSH secreting cells.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, and composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

1.-40. (canceled)

41. An in vitro method for inducing differentiation of pituitary precursors, comprising contacting cells expressing at least one pituitary precursor marker with at least one dorsalizing agent and at least one ventralizing agent; and contacting the cells with at least one activator of Wingless (Wnt) signaling (Wnt activator) to obtain a cell population of differentiated cells, wherein at least 50% of differentiated cells express pituitary-specific transcription factor (Pit1), wherein the at least one dorsalizing agent comprises at least one activator of FGF signaling, and wherein the at least one ventralizing agent comprises at least one BMP molecule.

42. The method of claim 41, comprising obtaining the cell population at least 5 days, at least 7 days, or at least 11 days from the initial contact of the cells expressing at least one pituitary precursor marker with the at least one dorsalizing agent.

43. The method of claim 41, comprising contacting stem cells with at least one BMP molecule and at least one inhibitor of TGFβ/Activin-Nodal signaling; and contacting the cells with at least one activator of Sonic Hedgehog (SHH) signaling, and at least one activator of FGF signaling to obtain the cells expressing at least one pituitary precursor marker.

44. The method of claim 43, comprising obtaining the cell population at least 10 days, at least 15 days, 15 days, or 20 days from the initial contact of the stem cells with the at least one inhibitor of TGFβ/Activin-Nodal signaling.

45. The method of claim 41, wherein
a) the initial contact of the cells with the at least one Wnt activator is no later than 5 days from, about 4 days from, or on the same day as the initial contact of the cells expressing at least one pituitary precursor marker with the at least one dorsalizing agent; and/or
b) the cells are contacted with the at least one Wnt activator for at least 5 days, at least 15 days, or at least 7 days; and/or
c) the cells are contacted with the at least one dorsalizing agent for at least 3 days, at least 10 days, at least 4 days, or at least 7 days; and/or
d) the cells are contacted with the at least one ventralizing agent for at least 5 days, at least 15 days, at least 7 days, or at least 11 days.

46. The method of claim 41, further comprising contacting the cells expressing at least one pituitary precursor marker with at least one inhibitor of TGFβ/Activin-Nodal signaling, optionally wherein
a) the at least one inhibitor of TGFβ/Activin-Nodal signaling is contacted with the cells for at least 3 days, at least 10 days, at least 4 days, or at least 7 days; and/or
b) the cells are contacted with the at least one dorsalizing agent and the at least one inhibitor of TGFβ/Activin-Nodal signaling concurrently.

47. The method of claim 41, further comprising contacting the cells with at least one estrogen receptor (ER) agonist, optionally wherein
a) the cells are contacted with at least one ER agonist and the at least one Wnt activator concurrently; and/or
b) the initial contact of the cells with the at least one ER agonist is no later than 10 days from, or about 4 days from, or occurs on the same day as the initial contact of the cells expressing at least one pituitary precursor marker with the at least one dorsalizing agent; and/or
c) the cells are contacted with the at least one ER agonist for at least 5 days, at least 15 days, at least 7 days, or at least 8 days.

48. The method of claim 43, wherein
a) the stem cells are contacted with the at least one BMP molecule for at least 2 days; and/or
b) the stem cells are contacted with the at least one inhibitor of TGFβ/Activin-Nodal signaling for at least 5 days; and/or
c) the stem cells are contacted with the at least one activator of Sonic Hedgehog (SHH) signaling and one, two, or more activator of FGF signaling for at least 2 days; and/or
d) further comprising contacting the stem cells with at least one inhibitor of Small Mothers Against Decapentaplegic (SMAD) signaling, optionally wherein
i) the stem cells are contacted with the at least one inhibitor of SMAD signaling for at least 2 days; and/or
ii) the at least one inhibitor of SMAD signaling is selected from the group consisting of LDN193189, Noggin, Dorsomorphin, K02288, dorsomorphin homolog 1 (DMH1), ML347, LDN 212854, derivatives thereof, and mixtures thereof, optionally wherein the at least one inhibitor of SMAD signaling comprises LDN193189; and/or
iii) the cells are contacted with the at least one inhibitor of SMAD signaling at a concentration of between 100 nM and 300 nM.

49. The method of claim 41, comprising differentiating the cells expressing at least one pituitary precursor marker to cells expressing Pituitary-specific transcription factor (Pit1), differentiating the cells expressing Pit1 to GHRHR^{low} cells, and differentiating the GHRHR^{low} cells to GHRHR^{high} cells.

50. The method of claim 49, wherein differentiation of the cells expressing Pit1 to GHRHR^{low} cells comprises contacting cells expressing Pit1 with a first combination of GH inducers.

51. The method of claim 50, wherein the first combination of GH inducers are selected from the group consisting of retinoic acid (RA), corticosteroids, thyroid hormones, GHRH signaling agonists, ER agonists, and Ghrelin signaling pathway agonists, optionally wherein
a) the first combination of GH inducers comprises retinoic acid (RA), a corticosteroid, a thyroid hormone, and a GHRH signaling agonist, optionally wherein the first combination of GH inducers comprises RA, dexamethasone, L-triiodothyronine (T3), and GHRH; or
b) the first combination of GH inducers comprises retinoic acid (RA), a corticosteroid, a thyroid hormone, two GHRH signaling agonists, and an ER agonist, optionally wherein the first combination of GH inducers comprises RA, dexamethasone, T3, GHRH, CAMP, and diarylpropionitrile (DPN); or
c) the first combination of GH inducers comprises retinoic acid (RA), a corticosteroid, a thyroid hormone, a GHRH signaling agonist, an ER agonist, and a Ghrelin signaling pathway agonist; optionally wherein the first combination of GH inducers comprises RA, dexamethasone, T3, GHRH, DPN, and Ghrelin.

52. The method of claim 50, wherein the cells expressing Pit1 are contacted with the first combination of GH inducers for at least 3 days, at least 5 days, or at least two weeks to obtain a cell population comprising at least about-50% GHRHR^{low} cells.

53. The method of claim 49, wherein differentiation of the GHRHR^{low} cells to GHRHR^{high} cells comprises contacting the GHRHR^{low} cells with a second combination of GH inducers.

54. The method of claim 53, wherein the second combination of GH inducers are selected from the group consisting of retinoic acid (RA), corticosteroids, thyroid hormones, GHRH signaling agonists, ER agonists, interleukins, and Ghrelin signaling pathway agonists, optionally wherein
a) the second combination of GH inducers comprises retinoic acid (RA), a corticosteroid, a thyroid hormone, and a GHRH signaling agonist; optionally wherein the second combination of GH inducers comprises RA, dexamethasone, L-triiodothyronine (T3), and GHRH; or
b) the second combination of GH inducers comprises retinoic acid (RA), a corticosteroid, a thyroid hormone, two GHRH signaling agonists, and an ER agonist; optionally wherein the second combination of GH inducers comprises RA, dexamethasone, T3, GHRH, CAMP, and diarylpropionitrile (DPN); or
c) the second combination of GH inducers comprises a corticosteroid, a thyroid hormone, a GHRH signaling agonist, an ER agonist, an interleukin, and a Ghrelin signaling pathway agonist; optionally wherein the second combination of GH inducers comprises dexamethasone, T3, GHRH, IL-6, Ghrelin, and DPN.

55. The method of claim 53, wherein the GHRHR^{low} cells are contacted with the second combination of GH inducers for at least 5 days, at least 10 days, or at least four weeks to obtain a cell population comprising at least 50% GHRHR^{high} cells.

56. The method of claim 41, wherein the at least one pituitary precursor marker is selected from the group consisting of Sine oculis homeobox homolog 1 (SIX1), LIM/homeobox protein LHX3, LIM/homeobox protein LHX4, Pituitary homeobox 1 (PITX1), Pituitary homeobox 2 (PITX2), HESX homeobox 1 (HESX1), PROP paired-like homeobox 1 (PROP1), Sine oculis homeobox homolog 6 (SIX6), T-Box Transcription Factor 19 (TBX19), Paired box 6 (PAX6), GATA binding protein 2 (GATA2), Splicing Factor 1 (SF1), and combinations thereof

57. The method of claim 41, wherein the at least one activator of FGF signaling is selected from the group consisting of FGF1, FGF2, FGF3, FGF4, FGF7, FGF8, FGF10, FGF18, derivatives thereof, and mixtures thereof, optionally wherein the at least one activator of FGF signaling comprises a) FGF8, b) FGF8 and FGF10, and/or c) FGF8, FGF10, and FGF18.

58. The method of claim 41, wherein
i) the at least one BMP molecule is selected from the group consisting of BMP1, BMP2, BMP3, BMP4, BMPS, BMP6, BMP7, BMP8a, BMP8b, BMP10, BMP11, BMP15, derivatives thereof, and mixtures thereof, optionally wherein the at least one BMP molecule comprises BMP2 and/or BMP4; and
ii) the at least one Wnt activator is selected from the group consisting of CHIR99021, Wnt-1, WNT3A, Wnt4, Wnt5a, WAY-316606, IQ1, QS11, SB-216763, BIO (6-bromoindirubin-3′-oxime), LY2090314, Deoxycholic Acid (DCA), 2-amino-4-[3,4-(methylenedioxy)benzyl-amino]-6-β-methoxyphenyl)pyrimidine, (hetero)arylpyrimidines, derivatives thereof, and combinations thereof, optionally wherein the at least one Wnt activator comprises CHIR99021.

59. The method of claim 47, wherein the cells are contacted with the at least one ER agonist at a concentration of between about 0.1 nM and 20 nM.

60. The method of claim 54, wherein the cells are contacted with RA at a concentration of from about 0.1 μM to 1 μM.
